(19) United States
(12) Patent Application Publication
(10) Pub. No.: US 2026/0091089 A1
(43) Pub. Date: Apr. 2, 2026

(54) Pyocyanine Demethylases and related Phenazine Degrading Agents Compositions, Methods and Systems for Interfering with Viability of Bacteria

(71) Applicants: CALIFORNIA INSTITUTE OF TECHNOLOGY, Pasadena, CA (US); YEDA RESEARCH & DEVELOPMENT CO. LTD. AT THE WEIZMANN INSTITUTE OF SCIENCE, Rehovot (IL)

(72) Inventors: Dianne K. Newman, Pasadena, CA (US); Chelsey M. VanDrisse, VanDrisse, CA (US); Rosalie Lipsh-Sokolik, Rehovot (IL); Olga Khersonsky, Rehovot (IL); Sarel J. Fleishman, Rehovot (IL)

(21) Appl. No.: 19/312,182

(22) Filed: Aug. 27, 2025

Related U.S. Application Data
(62) Division of application No. 17/520,345, filed on Nov. 5, 2021, now Pat. No. 12,427,188.
(60) Provisional application No. 63/110,246, filed on Nov. 5, 2020.

(51) Int. Cl. A61K 38/44; A61K 31/7036; A61P 31/04; C12N 9/02

(52) U.S. Cl. CPC A61K 38/44; A61K 31/7036; A61P 31/04; C12N 9/0004

(57) ABSTRACT

Provided herein are engineered pyocyanin demethylases having replacements in in positions A53, 173, A87, T91, M99, A129 and K141 of pyocyanin demethylase PodA of SEQ ID NO: 1 or a derivative thereof and related phenazine degrading agents, compositions, methods and systems, as well as a combined administration of one or more pyocyanin demethylases and antibiotics and/or antibiotics resulting in a synergic inhibition of viability of phenazine producing bacteria, and related phenazine degrading agents, compositions, methods and systems.

### CROSS-REFERENCE TO RELATED APPLICATION

The present divisional application claims priority to U.S. application Ser. No. 17/520,345 filed on Nov. 5, 2021 entitled “Pyocyanin Demethylases and related Phenazine Degrading Agents Compositions, Methods and Systems for Interfering with Viability of Bacteria”, which in turn claims priority to U.S. Provisional Application No. 63/110,246 entitled “Phenazine Degrading Agents and Related Compositions, Methods, Systems for Interfering with Viability of Bacteria” filed on Nov. 5, 2020, with docket number CIT 8548-P, the contents of all of which are incorporated herein by reference in their entirety. The present applications may also be related to U.S. Non-Provisional application Ser. No. 15/466,839 entitled “Phenazine Degrading Agents And Related Compositions, Methods And Systems For Interfering With Viability Of Bacteria” filed on Mar. 22, 2017 and granted on Feb. 9, 2019 with US patent number U.S. Pat. No. 10,913,936, herein also incorporated herein by reference in its entirety.

### STATEMENT OF GOVERNMENT GRANT

This invention was made with government support under Grant No(s). AI127850 & HL152190 awarded by the National Institutes of Health. The government has certain rights in the invention.

### FIELD

The present disclosure relates to methods and systems for interfering with the viability of bacteria and related compounds and compositions. In particular, the present disclosure relates to a pyocyanin demethylase and related phenazine degrading agents, compositions, methods and systems for interfering with the viability of bacteria.

### REFERENCE TO SEQUENCE LISTING

Further, the computer readable form of the sequence listing of the XML text file P2655-USD-Seq-ID, created on Aug. 27, 2025 and having size of 12,399 bytes measured in Windows Server 2019 Datacenter, is incorporated herein by reference in its entirety.

### BACKGROUND

Phenazines are bacteria produced compounds which have been shown to be important for viability of phenazine producing bacteria

Whether for medical application or for fundamental biology studies, several methods are commonly used for the detection of and interference with the viability of bacteria. in particular when aimed at therapeutic or diagnostic applications.

Although methods, systems and compositions have been developed to interfere with, and in particular, to reduce bacterial viability through phenazine degradation, development of more efficacious phenazine degraders able to effectively inhibit bacterial viability is still particularly challenging.

### SUMMARY

Provided herein, are pyocyanin demethylases, related phenazine degrading agents, effective phenazine degrading agents' amounts, as well as related methods, systems and compositions that in several embodiments are configured for effective reduction of antibiotic resistance and/or survivability of phenazine producing bacteria.

In particular, provided herein are engineered pyocyanin demethylases which are based on the surprising identification of replacement in in positions A53, 173, A87, T91, M99, A129 and K141 of pyocyanin demethylase PodA having sequence

(SEQ ID NO: 1)
M₁T₂G₃K₄T₅K₆P₇A₈I₉I₁₀G₁₁G₁₂V₁₃A₁₄I₁₅T₁₆A₁₇L₁₈
A₁₉A₂₀A₂₁G₂₂L₂₃G₂₄V₂₅W₂₆L₂₇F₂₈T₂₉D₃₀G₃₁R₃₂G₃₃
G₃₄R₃₅S₃₆T₃₇T₃₈E₃₉P₄₀V₄₁T₄₂M₄₃T₄₄L₄₅D₄₆V₄₇K₄₈
N₄₉D₅₀Q₅₁V₅₂A₅₃K₅₄H₅₅D₅₆F₅₇G₅₈K₅₉P₆₀G₆₁M₆₂D₆₃
V₆₄G₆₅D₆₆M₆₇D₆₈I₆₉F₇₀S₇₁D₇₂I₇₃L₇₄S₇₅V₇₆D₇₇G₇₈
K₇₉Q₈₀V₈₁G₈₂Y₈₃D₈₄G₈₅G₈₆A₈₇C₈₈F₈₉F₉₀T₉₁N₉₂V₉₃
T₉₄P₉₅D₉₆N₉₇P₉₈M₉₉T₁₀₀Y₁₀₁C₁₀₂E₁₀₃L₁₀₄T₁₀₅I₁₀₆
H₁₀₇L₁₀₈D₁₀₉A₁₁₀G₁₁₁E₁₁₂I₁₁₃F₁₁₄A₁₁₅R₁₁₆S₁₁₇
L₁₁₈T₁₁₉P₁₂₀H₁₂₁T₁₂₂L₁₂₃A₁₂₄P₁₂₅F₁₂₆T₁₂₇M₁₂₈
A₁₂₉I₁₃₀T₁₃₁G₁₃₂G₁₃₃T₁₃₄G₁₃₅E₁₃₆Y₁₃₇A₁₃₈N₁₃₉
S₁₄₀K₁₄₁G₁₄₂E₁₄₃L₁₄₄T₁₄₅V₁₄₆S₁₄₇G₁₄₈V₁₄₉A₁₅₀
T₁₅₁P₁₅₂D₁₅₃E₁₅₄K₁₅₅Y₁₅₆E₁₅₇L₁₅₈K₁₅₉L₁₆₀T₁₆₁
K₁₆₂,

which can increase stability, yield and/or activity of the pyocyanin demethylase in SEQ ID NO: 1 or portions thereof, as well as combined administration of natural occurring and/or engineered pyocyanin demethylases and antibiotics resulting in a synergic inhibition of viability of phenazine producing bacteria.

According to a first aspect, an engineered pyocyanin demethylase or a derivative thereof are described, the pyocyanin demethylase comprising sequence

(SEQ ID NO: 1)
M₁T₂G₃K₄T₅K₆P₇A₈I₉I₁₀G₁₁G₁₂V₁₃A₁₄I₁₅T₁₆A₁₇L₁₈
A₁₉A₂₀A₂₁G₂₂L₂₃G₂₄V₂₅W₂₆L₂₇F₂₈T₂₉D₃₀G₃₁R₃₂G₃₃
G₃₄R₃₅S₃₆T₃₇T₃₈E₃₉P₄₀V₄₁T₄₂M₄₃T₄₄L₄₅D₄₆V₄₇K₄₈
N₄₉D₅₀Q₅₁V₅₂A₅₃K₅₄H₅₅D₅₆F₅₇G₅₈K₅₉P₆₀G₆₁M₆₂D₆₃
V₆₄G₆₅D₆₆M₆₇D₆₈I₆₉F₇₀S₇₁D₇₂I₇₃L₇₄S₇₅V₇₆D₇₇G₇₈
K₇₉Q₈₀V₈₁G₈₂Y₈₃D₈₄G₈₅G₈₆A₈₇C₈₈F₈₉F₉₀T₉₁N₉₂V₉₃
T₉₄P₉₅D₉₆N₉₇P₉₈M₉₉T₁₀₀Y₁₀₁C₁₀₂E₁₀₃L₁₀₄T₁₀₅I₁₀₆
H₁₀₇L₁₀₈D₁₀₉A₁₁₀G₁₁₁E₁₁₂I₁₁₃F₁₁₄A₁₁₅R₁₁₆S₁₁₇
L₁₁₈T₁₁₉P₁₂₀H₁₂₁T₁₂₂L₁₂₃A₁₂₄P₁₂₅F₁₂₆T₁₂₇M₁₂₈
A₁₂₉I₁₃₀T₁₃₁G₁₃₂G₁₃₃T₁₃₄G₁₃₅E₁₃₆Y₁₃₇A₁₃₈N₁₃₉
S₁₄₀K₁₄₁G₁₄₂E₁₄₃L₁₄₄T₁₄₅V₁₄₆S₁₄₇G₁₄₈V₁₄₉A₁₅₀
T₁₅₁P₁₅₂D₁₅₃E₁₅₄K₁₅₅Y₁₅₆E₁₅₇L₁₅₈K₁₅₉L₁₆₀
T₁₆₁K₁₆₂,

the pyocyanin demethylase further modified to include at least two replacements in SEQ ID NO: 1 selected from
A53 with L, N, or V
I73 with I, E, K, L, Q, R, T or V
A87 with C, I, T or V
T91 with V
M99 with M, C, F, I, K, R, V or Y
A129 with A, C, S, T, or V
K141 with K, S or T
   and configured to be capable of the engineered pyocyanin demethylase or a derivative thereof to demethylate pyocyanin and/or a pyocyanin-like phenazines of formula (III)

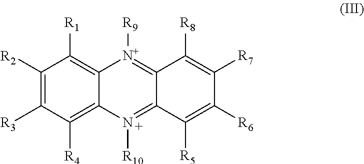

where R₁-R₁₀ are independently selected from hydrogen, hydroxy, alkoxy, alkyl, alkenyl, alkynyl, aryl, heteroaryl, acyl, and other groups identifiable to the skilled person, and one of R₁-R₁₀ is a negatively charged substituent (formal charge of −1) such as

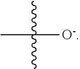

According to a second and preferred aspect, an engineered pyocyanin demethylase PodA₃₀₋₁₆₂ or a derivative thereof are described, the pyocyanin demethylase comprising sequence

(SEQ ID NO: 2)
D₁G₂R₃G₄G₅R₆S₇T₈T₉E₁₀P₁₁V₁₂T₁₃M₁₄T₁₅L₁₆D₁₇
VI₈K₁₉N₂₀D₂₁Q₂₂V₂₃A₂₄K₂₅H₂₆D₂₇F₂₈G₂₉K₃₀P₃₁
G₃₂M₃₃D₃₄V₃₅G₃₆D₃₇M₃₈D₃₉I₄₀F₄₁S₄₂D₄₃I₄₄L₄₅
S₄₆V₄₇D₄₈G₄₉K₅₀Q₅₁A₅₂G₅₃Y₅₄D₅₅G₅₆G₅₇A₅₈C₅₉
F₆₀F₆₁T₆₂N₆₃V₆₄T₆₅P₆₆D₆₇N₆₈P₆₉M₇₀T₇₁Y₇₂C₇₃
E₇₄L₇₅T₇₆I₇₇H₇₈L₇₉D₈₀A₈₁G₈₂E₈₃I₈₄F₈₅A₈₆R₈₇
S₈₈L₈₉T₉₀P₉₁H₉₂T₉₃L₉₄A₉₅P₉₆F₉₇T₉₈M₉₉A₁₀₀
I₁₀₁T₁₀₂G₁₀₃G₁₀₄T₁₀₅G₁₀₆E₁₀₇Y₁₀₈A₁₀₉N₁₁₀
S₁₁₁K₁₁₂G₁₁₃E₁₁₄L₁₁₅T₁₁₆V₁₁₇S₁₁₈G₁₁₉V₁₂₀
A₁₂₁T₁₂₂P₁₂₃D₁₂₄E₁₂₅K₁₂₆Y₁₂₇E₁₂₈L₁₂₉K₁₃₀
L₁₃₁T₁₃₂K₁₃₃A₁₃₄E₁₃₅N₁₃₆L₁₃₇Y₁₃₈F₁₃₉Q₁₄₀,

the pyocyanin demethylase further modified to include at least two replacements in SEQ ID NO: 2 selected from
A24 with L, N, or V
I44 with I, E, K, L, Q, R, T or V
A58 with C, I, T or V
T62 with V
M70 with M, C, F, I, K, R, V or Y
A100 with A, C, S, T, or V
K112 with K, S or T
   and configured to be capable of the engineered pyocyanin demethylase or a derivative thereof to demethylate pyocyanin and/or a pyocyanin-like phenazines of formula (III)

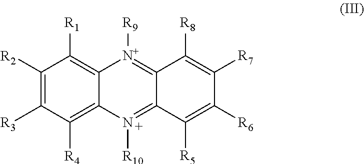

where R₁-R₁₀ are independently selected from hydrogen, hydroxy, alkoxy, alkyl, alkenyl, alkynyl, aryl, heteroaryl, acyl, and other groups identifiable to the skilled person, and one of R₁-R₁₀ is a negatively charged substituent (formal charge of −1) such as

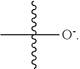

According to a third and further preferred aspect, an engineered pyocyanin demethylase PodA₄₁₋₁₆₂ or a derivative thereof are described, the pyocyanin demethylase comprising sequence

(SEQ ID NO: 3)
V₁T₂M₃T₄L₅D₆V₇K₈N₉D₁₀Q₁₁V₁₂A₁₃K₁₄H₁₅D₁₆F₁₇
G₁₈K₁₉P₂₀G₂₁M₂₂D₂₃V₂₄G₂₅D₂₆M₂₇D₂₈I₂₉F₃₀S₃₁
D₃₂I₃₃L₃₄S₃₅V₃₆D₃₇G₃₈K₃₉Q₄₀V₄₁G₄₂Y₄₃D₄₄G₄₅
G₄₆A₄₇C₄₈F₄₉F₅₀T₅₁N₅₂V₅₃T₅₄P₅₅D₅₆N₅₇P₅₈M₅₉
T₆₀Y₆₁C₆₂E₆₃L₆₄T₆₅I₆₆H₆₇L₆₈D₆₉A₇₀G₇₁E₇₂I₇₃
F₇₄A₇₅R₇₆S₇₇L₇₈T₇₉P₈₀H₈₁T₈₂L₈₃A₈₄P₈₅F₈₆T₈₇
M₈₈A₈₉I₉₀T₉₁G₉₂G₉₃T₉₄G₉₅E₉₆Y₉₇A₉₈N₉₉S₁₀₀
K₁₀₁G₁₀₂E₁₀₃L₁₀₄T₁₀₅V₁₀₆S₁₀₇G₁₀₈V₁₀₉A₁₁₀
T₁₁₁P₁₁₂D₁₁₃E₁₁₄K₁₁₅Y₁₆E₁₁₇L₁₁₈K₁₁₉L₁₂₀
T₁₂₁K₁₂₂,

the pyocyanin demethylase further modified to include at least two replacements in SEQ ID NO: 3 selected from
A13 with L, N, or V
I33 with I, E, K, L, Q, R, T or V
A47 with C, I, T or V
T51 with V
M59 with M, C, F, I, K, R, V or Y
A89 with A, C, S, T, or V
K101 with K, S or T
   and configured to be capable of the engineered pyocyanin demethylase or a derivative thereof to demethylate pyocyanin and/or a pyocyanin-like phenazines of formula (III)

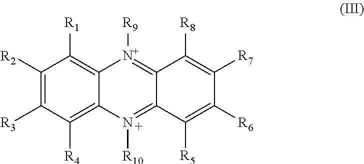

where R₁-R₁₀ are independently selected from hydrogen, hydroxy, alkoxy, alkyl, alkenyl, alkynyl, aryl, heteroaryl, acyl, and other groups identifiable to the skilled person, and one of R₁-R₁₀ is a negatively charged substituent (formal charge of −1) such as

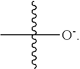

According to a fourth aspect, a phenazine degrading agent is described, comprising an engineered pyocyanin demethylase of the present disclosure and/or a derivative thereof.

According to a fifth aspect, a method and a system to interfere with viability of phenazine producing bacteria are described. The method comprises
contacting the phenazine producing bacteria with an engineered pyocyanin demethylase of the present disclosure and/or a derivative thereof, alone or in combination with an antibiotic and/or other antimicrobial for a time and under conditions to reduce survivability and/or antibiotic resistance of the bacteria.

In some embodiments contacting the engineered pyocyanin demethylase of the present disclosure and/or a derivative thereof, can be further performed in combination with other phenazine degrading agents possibly within a phenazine degrading agent according to the second aspect of the present disclosure.

The system comprises one or more engineered pyocyanin demethylase of the present disclosure and/or a derivative thereof, one or more phenazine degrading agents of the present disclosure, one or more antibiotics and/or one or more other antimicrobials. In some embodiments of the methods and systems, the bacteria comprise persister cells . . .

According to a sixth aspect, a method and a system are described for treating and/or preventing a bacterial infection by a phenazine producing bacteria in an individual. The method comprises
administering to the individual an effective amount of one or more an engineered pyocyanin demethylase of the present disclosure and/or a derivative thereof, alone or in combination with an antibiotic and/or another antimicrobial. In particular, in some embodiments, administering of an engineered pyocyanin demethylase of the present disclosure and/or a derivative thereof can be performed in combination with one or more antibiotics and/or other antimicrobials.

In some embodiments contacting the engineered pyocyanin demethylase of the present disclosure and/or a derivative thereof, can be further performed in combination with other phenazine degrading agents possibly within a phenazine degrading agent according to the second aspect of the present disclosure.

The system comprises one or more engineered pyocyanin demethylase of the present disclosure and/or a derivative thereof, one or more phenazine degrading agents, one or more-antibiotics and/or one or more other antimicrobials. In some embodiments of methods and systems, the bacteria comprise persister cells.

According to a seventh aspect, a method and a system to interfere with viability of phenazine producing bacteria are described. The method comprises
contacting the phenazine producing bacteria with
a phenazine degrading agent comprising a pyocyanin demethylase in an amount of at least 0.01 uM, preferably at least 0.2 uM and more preferably at least 0.5 uM and higher alone or more preferably in combination with
an antibiotic at a concentration of at least 2.0 ug mL, at least 10.0 ug mL-1, at least 25.0 ug mL-1, at least 50.0 ug mL-1, and at least 100.0 ug m
   the contacting performed for a time and under conditions to reduce survivability and/or antibiotic resistance of the bacteria.

In most preferred embodiments the phenazine degrading agent comprising a pyocyanin demethylase in an amount of at least 5 uM and possibly between 5 uM and 20 uM, and the antibiotic is preferably an aminoglycoside

In some embodiments, the phenazine degrading agent comprises or consists of at least one engineered pyocyanin demethylase of the present disclosure and/or a derivative thereof,

The system comprises one or more phenazine degrading agents in an amount of at least 0.01 uM, preferably at least 0.2 uM and more preferably at least 0.5 uM and higher and in most preferred embodiments at least 5 uM and possibly between 5 uM and 20 uM, and one or more antibiotics in an amount of at least 0.5 ug mL preferably from 40-60 ug mL depending on the MIC of the antibiotics as will be understood by a skilled person and/or one or more other antimicrobials. In some embodiments of the methods and systems, the bacteria comprise persister cells.

According to a eighth aspect, a method and a system are described for treating and/or preventing a bacterial infection by a phenazine producing bacteria in an individual. The method comprises
administering to the individual an effective amount of with phenazine degrading agent in an amount of at least 0.01 uM, preferably at least 5 uM and possibly between 5 uM and 20 uM alone or in combination with an antibiotic and/or other antimicrobials.

In some embodiments, the phenazine degrading agent comprises or consists of at least one engineered pyocyanin demethylase of the present disclosure and/or a derivative thereof,

The system comprises one or more phenazine degrading agents in an amount of at least 0.01 uM, preferably at least 5 uM and possibly between 5 uM and 20 uM one or more phenazine degrading agents, one or more antibiotics and/or one or more other antimicrobials. In some embodiments of methods and systems, the bacteria comprise persister cells.

According to a ninth aspect, an antimicrobial is described. The antimicrobial comprises one or more phenazine degrading agents herein described in an amount of at least 0.01 uM, preferably at least 5 uM and possibly between 5 uM and 20 uM and/or at least one engineered pyocyanin demethylase of the present disclosure and/or a derivative thereof.

In particular, the at least one engineered pyocyanin demethylase of the present disclosure and/or a derivative thereof are comprised in the antimicrobial in an amount suitable to reduce antibiotic resistance and/or survivability of phenazine producing bacteria and preferably in an amount of at least 0.01 uM, preferably at least 5 uM and possibly between 5 uM and 20 uM.

In some embodiments, the antimicrobial comprises a compatible vehicle, which can be a vehicle for effective administrating and/or delivering of the one or more agents to an individual. In some embodiments of the methods and systems, the bacteria comprise persister cells.

According to a tenth aspect, a composition is described. The composition comprises one or more phenazine degrading agents herein described in an amount of at least 0.01 uM, preferably at least 5 uM and possibly between 5 uM and 20 uM at least one engineered pyocyanin demethylase of the present disclosure and/or a derivative thereof, together with a compatible vehicle. In some embodiments, the composition can comprise one or more phenazine degrading agents and one or more medium components as will be understood by a skilled person.

According to an eleventh aspect, a method and system for inhibiting bacteria biofilm formation and/or disrupting mature biofilm in a medium is described. The method comprises administering an effective amount of 1-OH—PHZ as metal-chelating agent to the medium comprising the biofilm, alone or in combination with at least one engineered pyocyanin demethylase of the present disclosure and/or a derivative thereof, an antibiotic and/or other an antimicrobial for a time and under conditions to reduce survivability and/or antibiotic resistance of the bacteria.

The system comprises 1-OH—PHZ, one or more phenazine degrading agents herein described one or more antibiotics and/or one or more other antimicrobials.

The phenazine degrading agents and related antimicrobial compositions, methods and systems herein described, in several embodiments are expected to be particularly effective in treating and/or prevent bacterial infection in vitro or in vivo.

The phenazine degrading agents and related antimicrobial compositions, methods and systems herein described can be used in connection with applications wherein reduction of viability of bacteria and/or reduction of antibiotic resistance is desired, which include but are not limited to medical application, drug research, biological analysis and diagnostics including but not limited to clinical applications. Additional exemplary applications include uses of the methods and system and related compositions herein described in several fields including basic biology research, applied biology, bio-engineering, etiology, medical research, medical diagnostics, therapeutics, and in additional fields identifiable by a skilled person upon reading of the present disclosure.

### BRIEF DESCRIPTION OF THE DRA WINGS

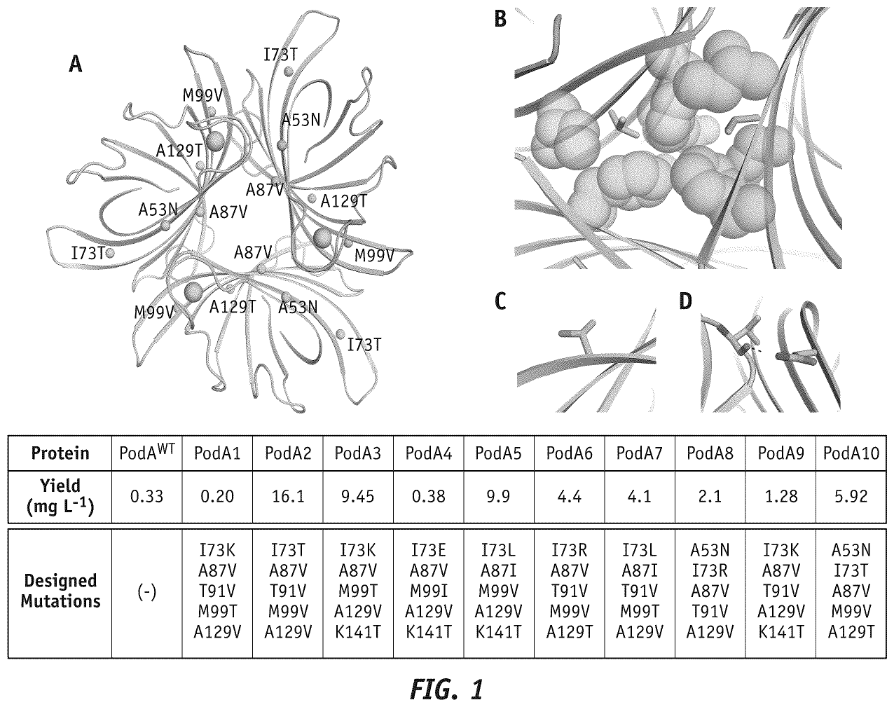

FIG. 1. Structure-based design of stabilized PodA variants. A) PodA forms a symmetric trimer. The structure depicted is a core structure of PodA₄₁₋₁₆₂ comprised in PodA₃₀₋₁₃₂, and PodA. The positions that were subjected to design are marked by shapes, with each monomer's amino acids subject to replacement indicated in the corresponding positions and each monomer of the trimer indicated with different shades of gray. B) Mutation Ala87Val increases core packing in a hydrophobic region. C) Mutation Ile73Thr improves surface polarity. D) Mutation Ala53Asn form an interfacial hydrogen bond with a Ser. E) Yield of trimer interface variants purified from inclusion bodies per L of overexpression culture. Amino acid residues changed for each variant are listed below yields.

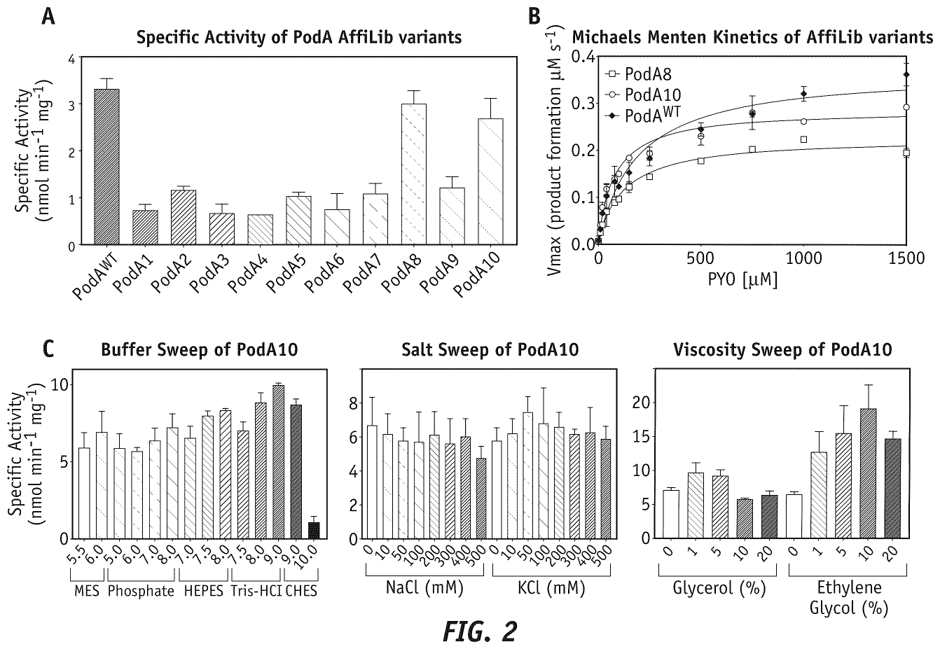

FIG. 2. Characterization of PodA10. A) PYO (50 M) demethylation activity of PodA variants (3 μM) was determined using a spectrophotometric assay described in the Materials and Methods. Error bars represent standard deviation of the mean of triplicates. B) Michaelis-Menten kinetics of PodA8 and PodA10 (3 μM) were found by plotting Vₘₐₓ values against [PYO], as indicated on x-axis. Error bars represent standard deviation of the mean of triplicates. C) PodA10 specific activity under varying conditions. PodA 10 (3 μM) was incubated with PYO (100 μM) and activity was monitored by measuring absorbance of PYO over time. Activity was analyzed by changing pH, viscosity, or salt concentrations. Error bars represent standard deviation of triplicates.

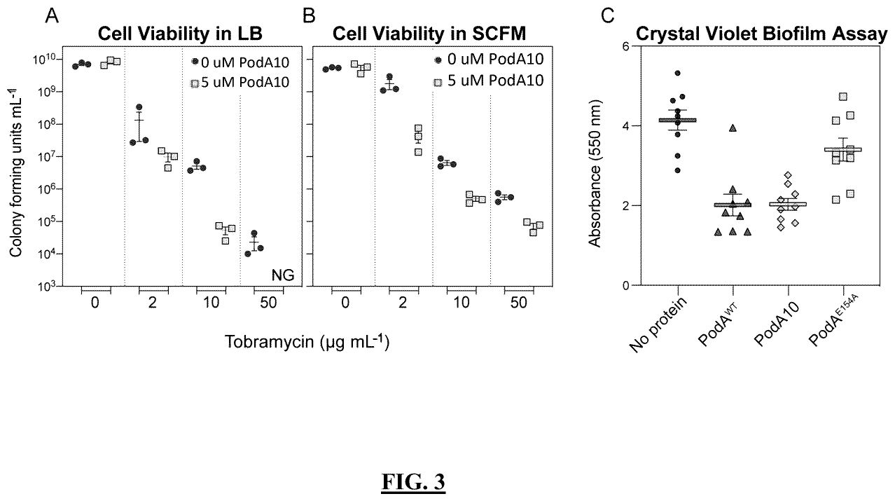

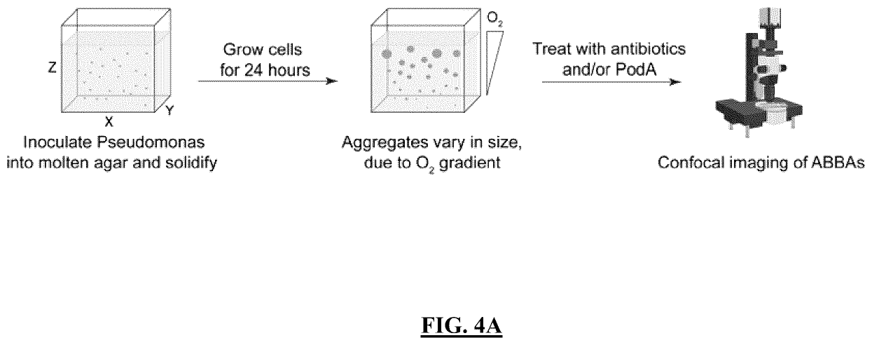

FIG. 3. Effects of PodA10 on planktonic viability and crude biofilm development. Cell viability counts of P. aeruginosa liquid medium synergy experiments with PodA 10 (0 μM or 5 μM) and tobramycin (concentrations indicated on x-axis) for LB (A) and SCFM (B). Data represent the mean of biological triplicates with error bars representing the standard error of the mean. NG; No Growth. (C) P. aeruginosa cells were inoculated in a 96-well polystyrene plate containing minimal medium with arginine (40 mM) and either no PodA, WT PodA₄₁₋₁₆₂, or PodA10, or inactive PodA^{E1544}, followed by incubation at 37° C. After 24 h of growth, planktonic cells were washed away, and sessile cells were stained with crystal violet. Cells were de-stained with an acetic acid and methanol mixture, after which sessile cells were quantified with relation to crystal violet absorbance at 550 nm. Error bars represent standard error of the mean.

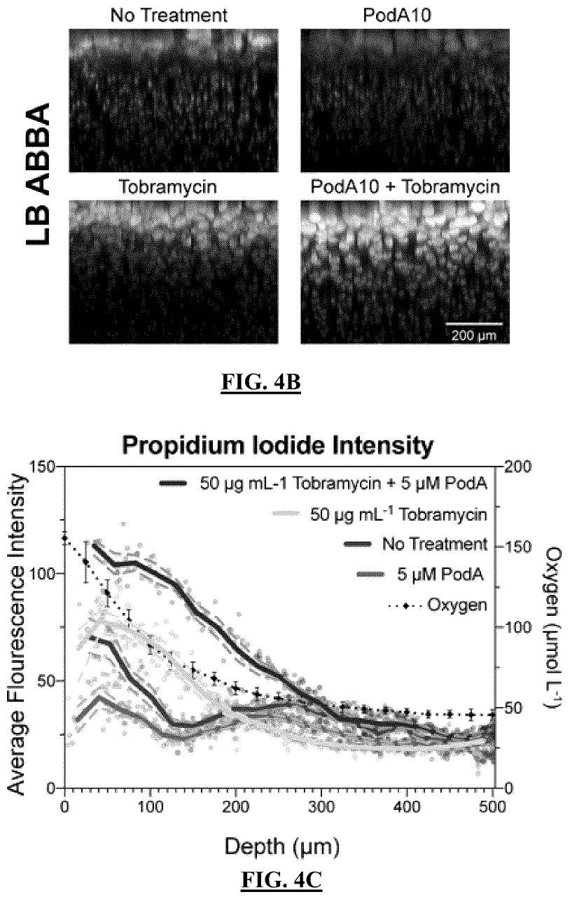

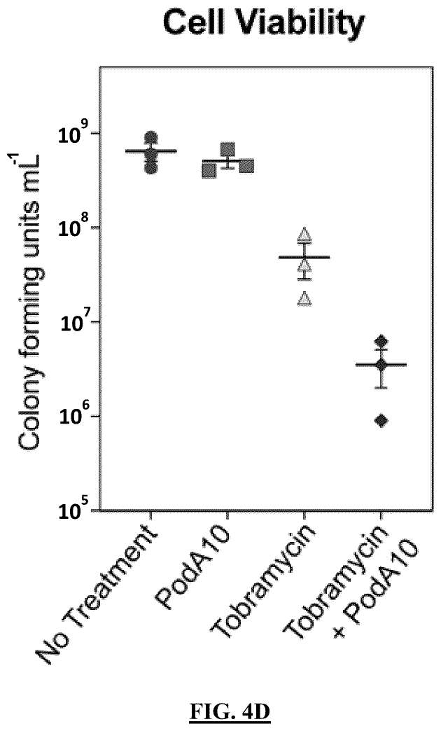

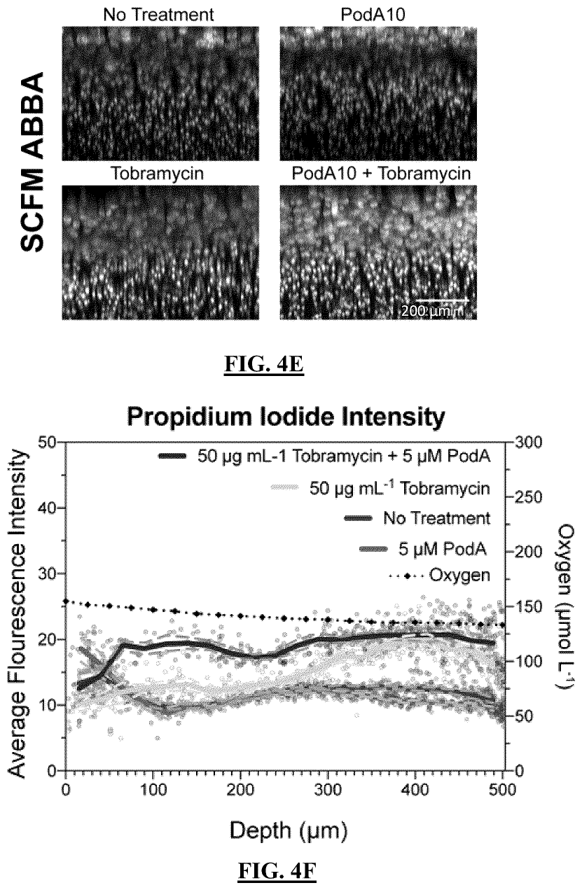

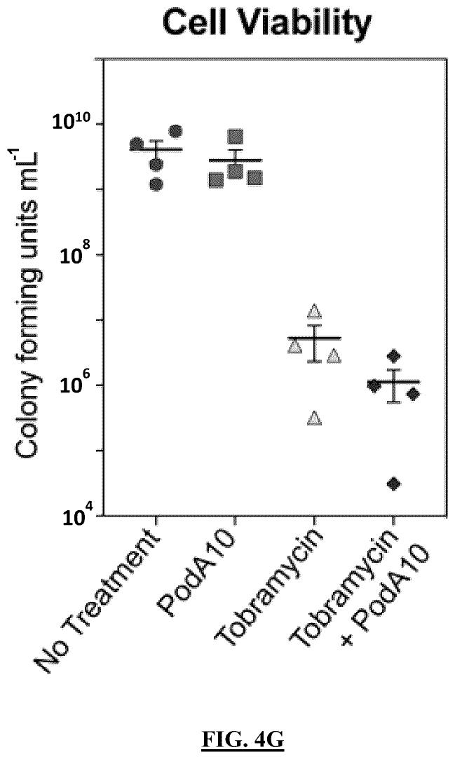

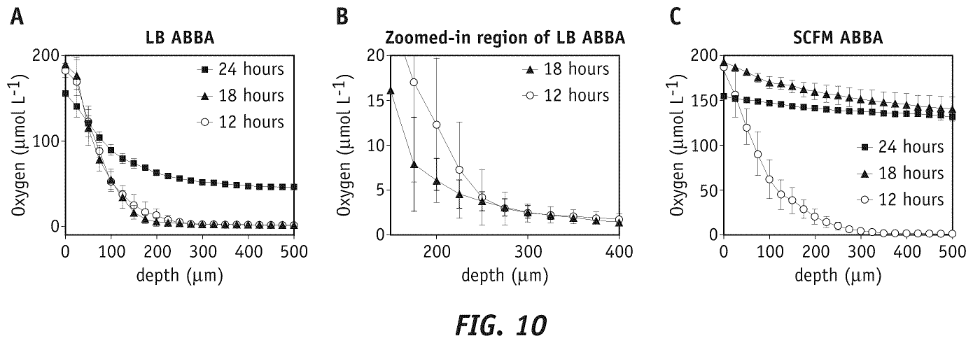

FIGS. 4A-4G. Microscopy imaging of propidium iodine stained ABBAs treated with PodA10 and tobramycin. Cells were incubated in an ABBA as described in Materials and Methods, followed by treatment with PodA10 (5 μM), tobramycin (50 μg mL⁻¹), or PodA10 and tobramycin. For no treatment conditions, buffer was added in lieu of protein. Cells were either grown in FIG. 4B) LB or FIG. 4E) SCFM. FIGS. 4B) and 4E) are presented as maximum-intensity XZ-projections and the brightness/contrast in each data set was normalized to the tobramycin only sample, as described in Materials and Methods. FIG. 4C) and 4F) represent an average pixel intensity (left Y-axis) of PI calculated from images in FIGS. 4B) and 4E). Each data point represents a single aggregate, with a bold lines representing the mean intensity and thin dashed lines representing the 95% confidence interval. Both mean and confidence interval were calculated as described in the Materials and Methods with a bin size of 20. Dotted black line represents average oxygen concentration measured after 24 hours as plotted on right Y-axis; 02 measurements after 12 and 18 hours are found in FIG. 10. Data points (diamonds) are the mean of technical triplicates of biological triplicates and error bars represent standard deviation. FIGS. 4D) and 4G) PodA and tobramycin ABBA treatments were homogenized, diluted in series, and dilutions were plated onto LB agar. Cells were counted if a dilution contained 10-100 colonies. Data represent the mean of biological triplicates with error bars representing the standard error of the mean. FIG. 4D) represents aggregates grown in LB and FIG. 4G) in SCFM. Data represent the mean of biological triplicates with error bars representing the standard error of the mean.

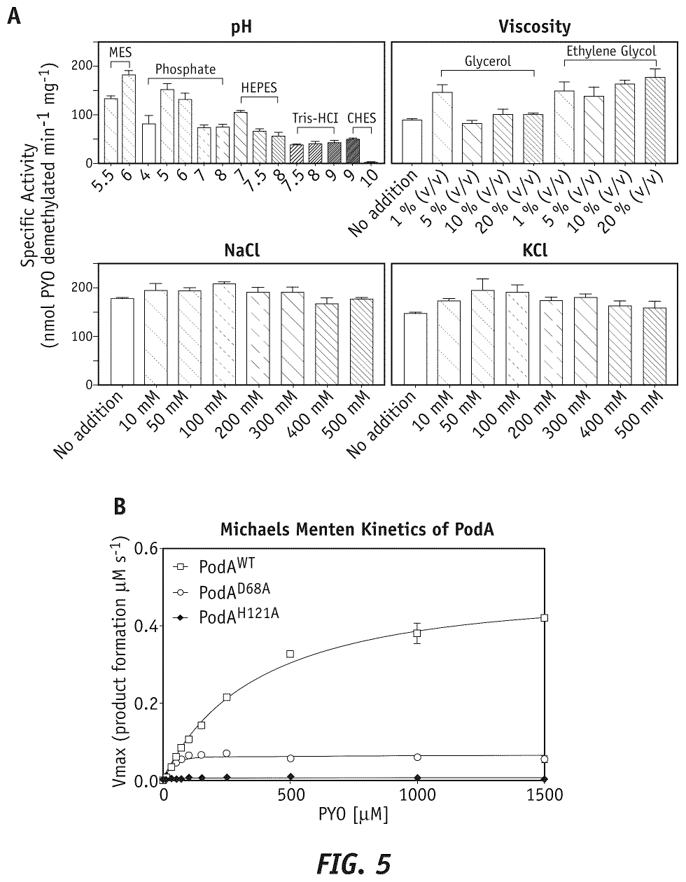

FIG. 5. Characterization of WT PodA₄₁₋₁₆₂ and active site mutant variants. (A) Specific activity of WT PodA₄₁₋₁₆₂ under varying conditions. WT PodA₄₁₋₁₆₂ (3 μM) was incubated with PYO (100 μM) and activity was monitored by measuring absorbance of PYO over time. Activity was analyzed by changing pH, viscosity, or salt concentrations. Error bars represent standard deviation of triplicates. (B) PodA (3 μM) and PYO (concentrations indicated on x-axis) were incubated and monitored at 690 nm. Vₘₐₓ values were plotted against [PYO] and an Ky was calculated by fitting data to the Michaelis-Menten equation as indicated by black line. Each data point represents the mean of three replicates and error bars represent standard deviation.

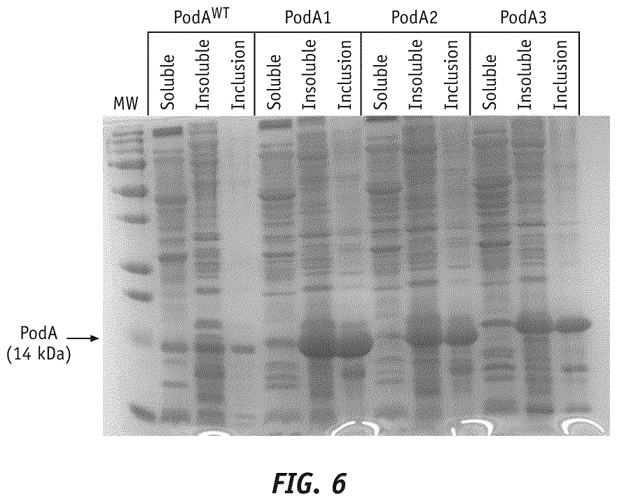

FIG. 6. Protein design of PodA trimer interface. Inclusion bodies from overexpression cultures of designed variants were analyzed using BugBuster™. A representative SDS-PAGE gel is shown for PodA1-PodA3. Precision Plus Protein™ used as a MW marker (lane 1).

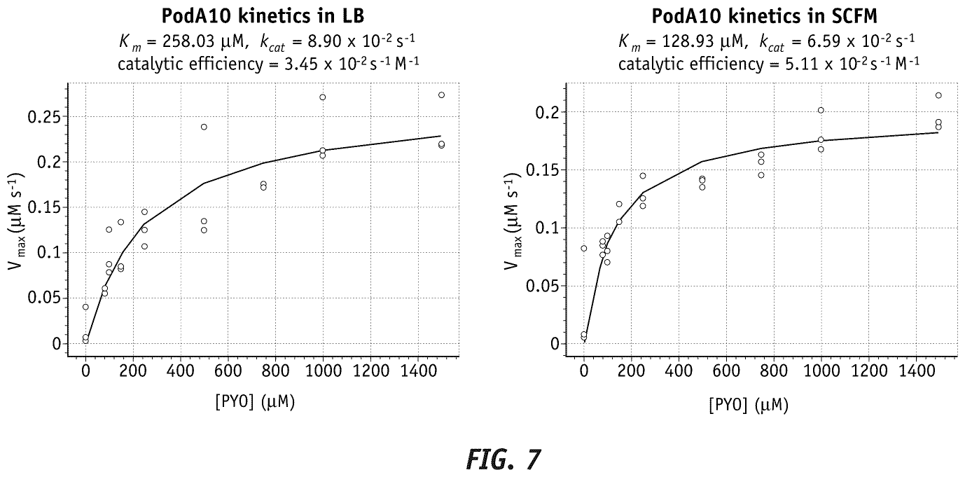

FIG. 7. Kinetics of PodA in growth media. Using a continuous spectrophotometric assay as described in the Material and Methods, PodA (3 μM) and PYO (concentrations indicated on x-axis) were incubated and monitored at 690 nm. Vₘₐₓ values were plotted against [PYO] and an K_{M} was calculated by fitting data to the Michaelis-Menten equation as indicated by black line. Each data point represents the mean of three replicates and error bars represent standard deviation. LB: lysogeny broth, SCFM; synthetic cystic fibrosis medium.

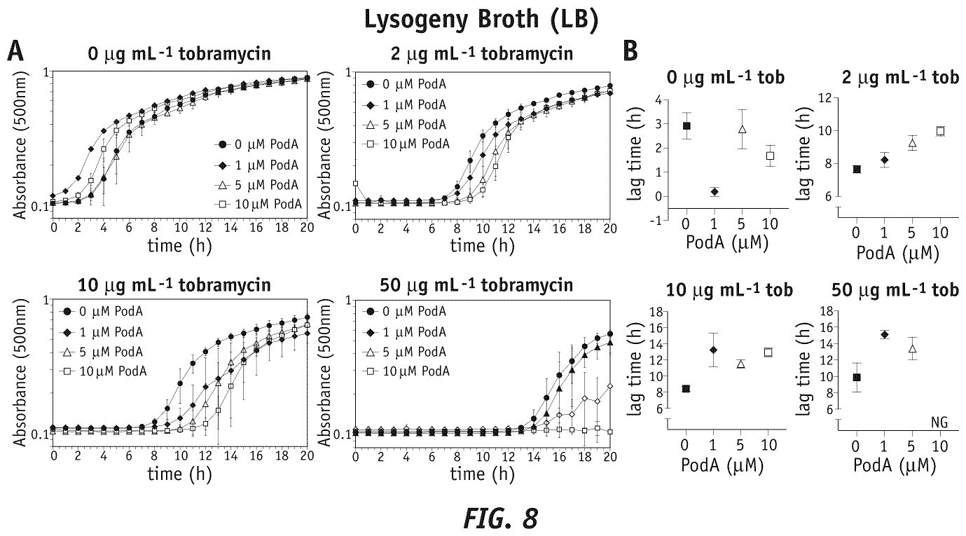

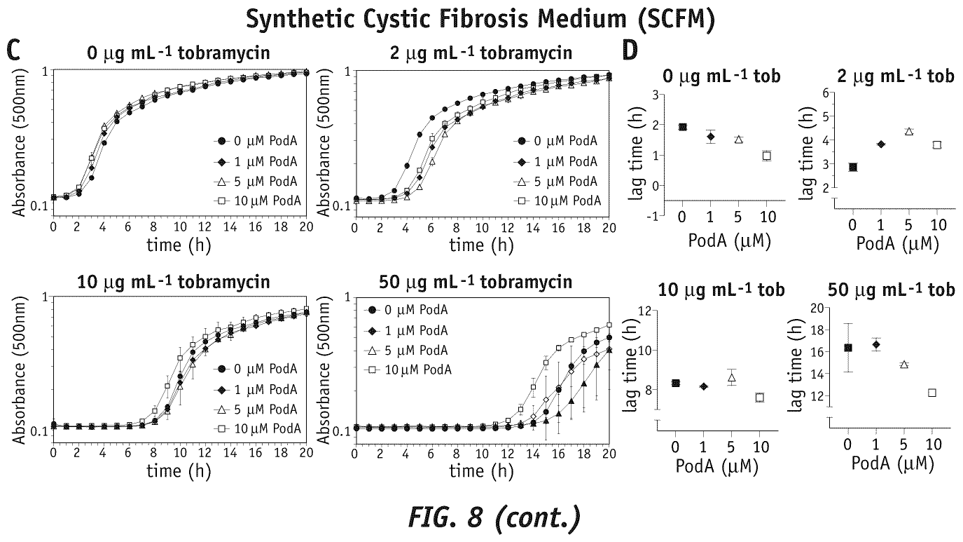

FIG. 8. Pseudomonas PodA and antibiotic synergy experiments in LB and SCFM. Growth curves represent outgrowth of cells that were incubated in LB (A) or SCFM (C) with PodA and no tobramycin (top left), or varying amounts of tobramycin (indicated in titles). Buffer was added in lieu of protein for 0 μM PodA controls. Growth was monitored at OD500 over time using a BioTek Plate Reader. Experiment was repeated in triplicate with a representative graph is shown. Error bars represent the standard error of the mean of triplicates. B) and D) Lag times of cultures were calculated from growth curves in respective panels. Data were fit to the Gompertz curve-fitting model using Growth Curve Fitting program in R (see website: webpage scott-h-saunders.shinyapps.io/gompertz_fitting_Ov2/). Data represent the mean and standard error of triplicates. NG; no growth.

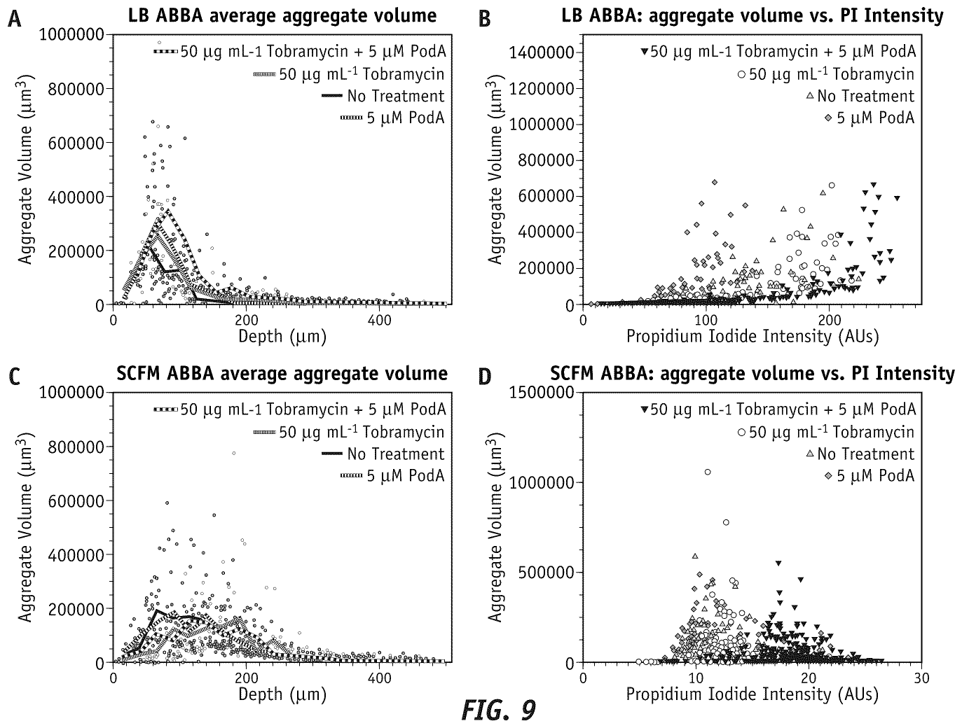

FIG. 9. Pseudomonas LB and SCFM ABBA aggregate volume analyses. ABBA data from FIGS. 4A-4G were analyzed in Imaris to obtain volume of each aggregate for LB (A) and SCFM (C). Mean aggregate volume was calculated with a bin size of 20 (every 25 μm) and plotted as a solid line (legend in each graph). Propidium Iodide intensity from FIGS. 4A-4G was plotted against each aggregate volume for LB (B) and SCFM (D).

FIG. 10. Pseudomonas LB and SCFM ABBA oxygen profiles. Cells were inoculated in LB or SCFM ABBAs and grown for the time indicated in graph legends. Oxygen profiles were determined using a micrometer oxygen sensor (UniSense). All time points consisted of biological triplicates with technical triplicate readings of each ABBA block with error bars representing standard deviation. A) Oxygen profile time course of LB ABBA. B) A zoomed in region of A) to display the transition from oxic to hypoxic (between 300-400 μm depths). C) Oxygen profile time course of SCFM ABBA.

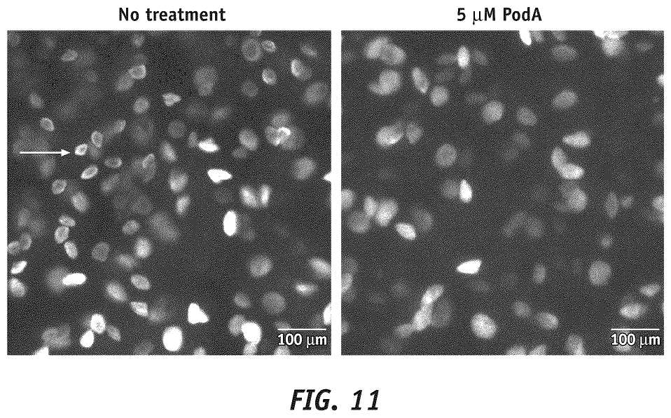

FIG. 11. Differences of PI staining in LB between untreated and PodA treated ABBAs. Microscopy data LB ABBAs were analyzed in the XY-plane at 90 μm depth (stack 15/84), with brightness contrast normalized to the no treatment control. Example of ring staining pattern indicated by red arrow.

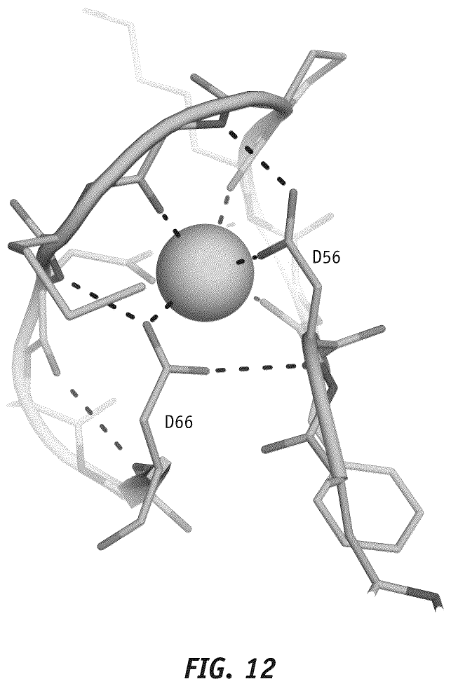

FIG. 12 (S2 PP). Ca²⁺ ion binding site of PodA. The loop between residues 56-66 connecting the ends of anti-parallel β-strands is stabilized through multiple bonds between a Ca²⁺ ion and loop backbone atoms and sidechains, mainly through Asp56 and Asp66. This Ca²⁺ ion was present in the crystal structure upon which our design calculations were based, likely due to the crystal having formed in a buffer that contained calcium [1].

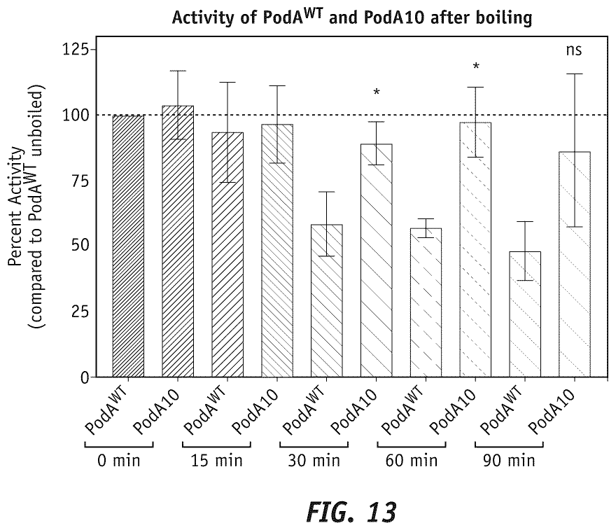

FIG. 13. Activity of WT PodA₃₀₋₁₆₂ and PodA10 after boiling. Protein was boiled as described in Materials and Methods, followed by an activity assay with PodA (3 μM) and PYO (100 μM) in a Spectramax M3 plate reader monitoring PYO absorbance (690 nm) overtime. Specific activity (nmol min-1 mg-1) was calculated for each sample. Replicates of PodAWT un-boiled were averaged and set to 100% activity (dashed line). All other activities of boiled samples were compared to this value and presented as a percentage. Data represent technical triplicates and error bars represent standard deviation. A t-test was completed, with * representing a p-value <0.05 and ns representing not significant.

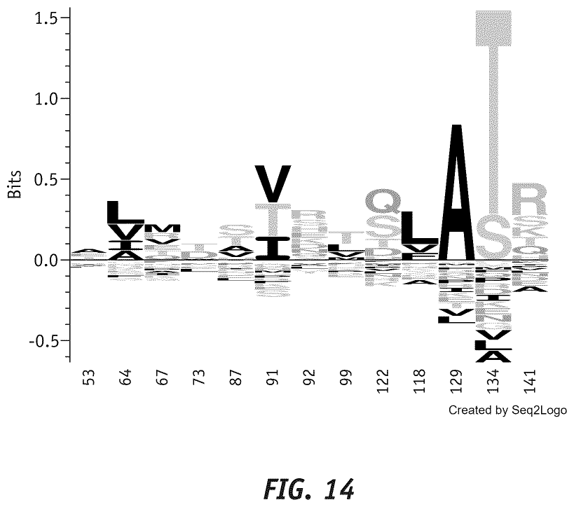

FIG. 14. Sequence logo of diversified positions. Sequence logo representing residue observations in evolutionary analysis, generated with default parameters using the indications in the web page.cbs.dtu.dk/biotools/Seq2Logo/. [2] Most successful designs did not use the most conserved residue in each position. For example, both PodA8 and PodA10 incorporated A53N, although Ala, Glu and Thr are evolutionarily more likely for this position. In addition, in position 129 the only preferable amino acid is Ala, but PodA8 and PodA10 mutated to A129V and A129T. respectively.

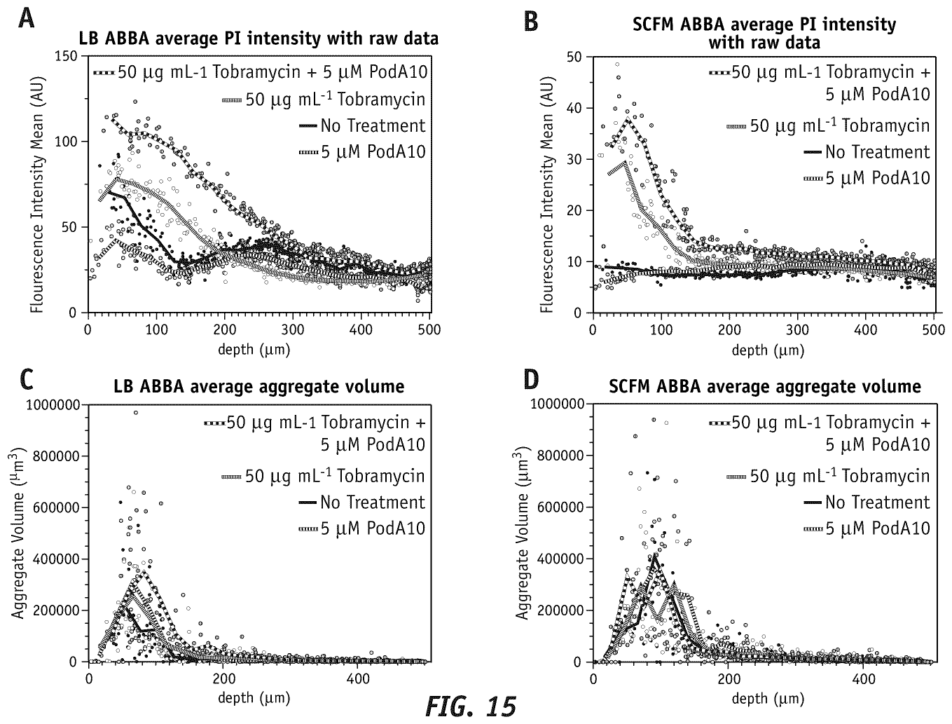

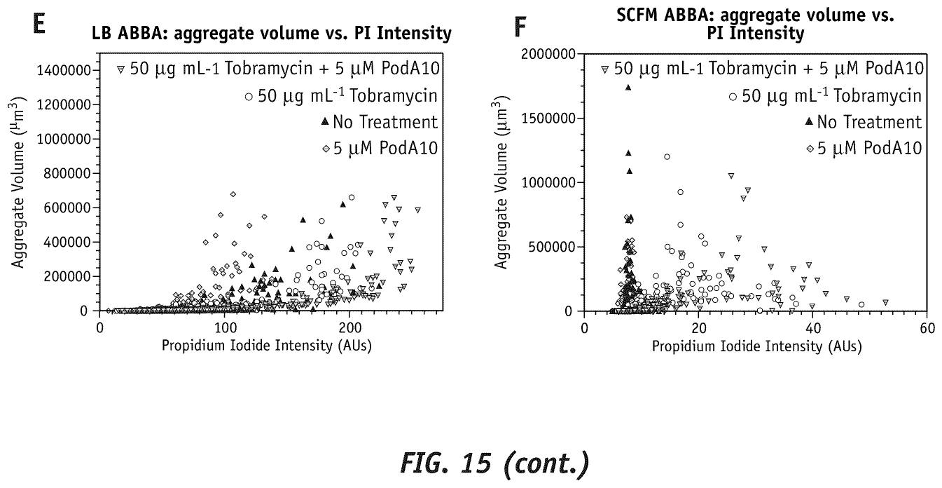

FIG. 15. Pseudomonas LB and SCFM ABBA aggregate volume analyses. ABBA data from FIGS. 4A-4G were analyzed in Imaris to obtain PI mean intensity for LB (A) and SCFM (B), along with the volume of each aggregate for LB (C) and SCFM (D). Each point represents a single aggregate. Mean aggregate volume was calculated with a bin size of 20 (every 25 μm) and plotted as a solid line. Propidium Iodide intensity from FIGS. 4A-4G was plotted against each aggregate volume for LB (E) and SCFM (F).

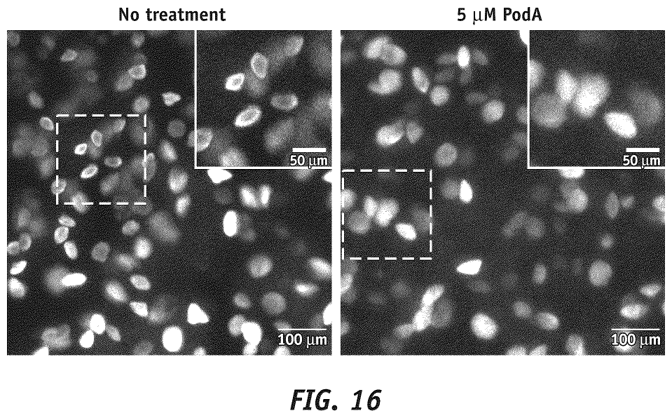

FIG. 16. Differences of PI staining in LB between untreated and PodA treated ABBAs. Microscopy data LB ABBAs were analyzed in the XY-plane at 90 μm depth (stack 15/84), with brightness contrast normalized to the no treatment control. Example of ring staining pattern indicated by zooming in on dashed boxes, with zoomed in images in the upper right corner. Look up table (LUT) settings were normalized to no treatment condition.

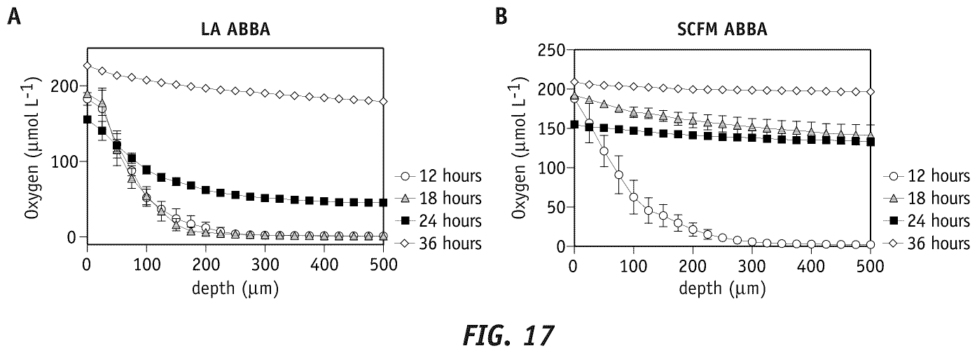

FIG. 17. Pseudomonas LB and SCFM ABBA oxygen profiles. Cells were inoculated in LB or SCFM ABBAs and grown for the time indicated in graph legends. Oxygen profiles were determined using a micrometer oxygen sensor (UniSense). All time points consisted of biological triplicates with technical triplicate readings of each ABBA block with error bars representing standard deviation. A) Oxygen profile time course of LB ABBA. B) Oxygen profile time course of SCFM ABBA.

### DETAILED DESCRIPTION

Provided herein are engineered phenazine degrading agents, effective phenazine degrading agents' amounts, as well as related methods, systems and compositions that in several embodiments are configured for effective reduction of antibiotic resistance and/or survivability of phenazine producing bacteria.

The term “phenazine” as used herein indicates small, colorful, redox-active compounds formed by bacteria to perform diverse physiological functions. In particular, “phenazines” in the sense of the disclosure comprise several phenazines of bacterial origin produced by bacteria such as Pseudomonas spp., Streptomyces spp., Burkholderia spp., and Pantoea agglomerans. The absorption spectra of phenazines are characteristic, with an intense peak in the range 250-290 nm and a weaker peak at 350-400 nm. At least one main band occurs in the visible region (400-600 nm) to which the phenazines owe their colors. Phenazines in the sense of the disclosure comprise compounds of Formula (I):

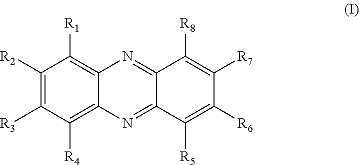

where R₁-R₈ are independently selected from hydrogen, hydroxy, alkoxy, alkyl, alkenyl, alkynyl, aryl, heteroaryl, acyl, and other groups identifiable to the skilled person.

Additionally, phenazines can include, but are not limited to, molecules according to the structures and formulas below:

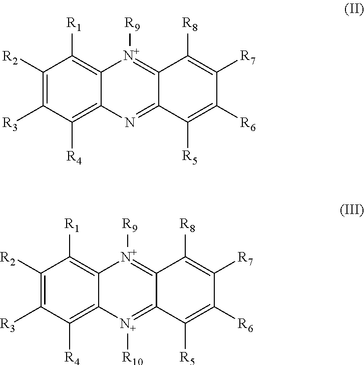

where R₁-R₁₀ are independently selected from hydrogen, hydroxy, alkoxy, alkyl, alkenyl, alkynyl, aryl, heteroaryl, acyl, and other groups identifiable to the skilled person, and one of R₁-R₁₀ is a negatively charged substituent (formal charge of −1) such as

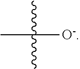

In particular, exemplary phenazine structures comprise:

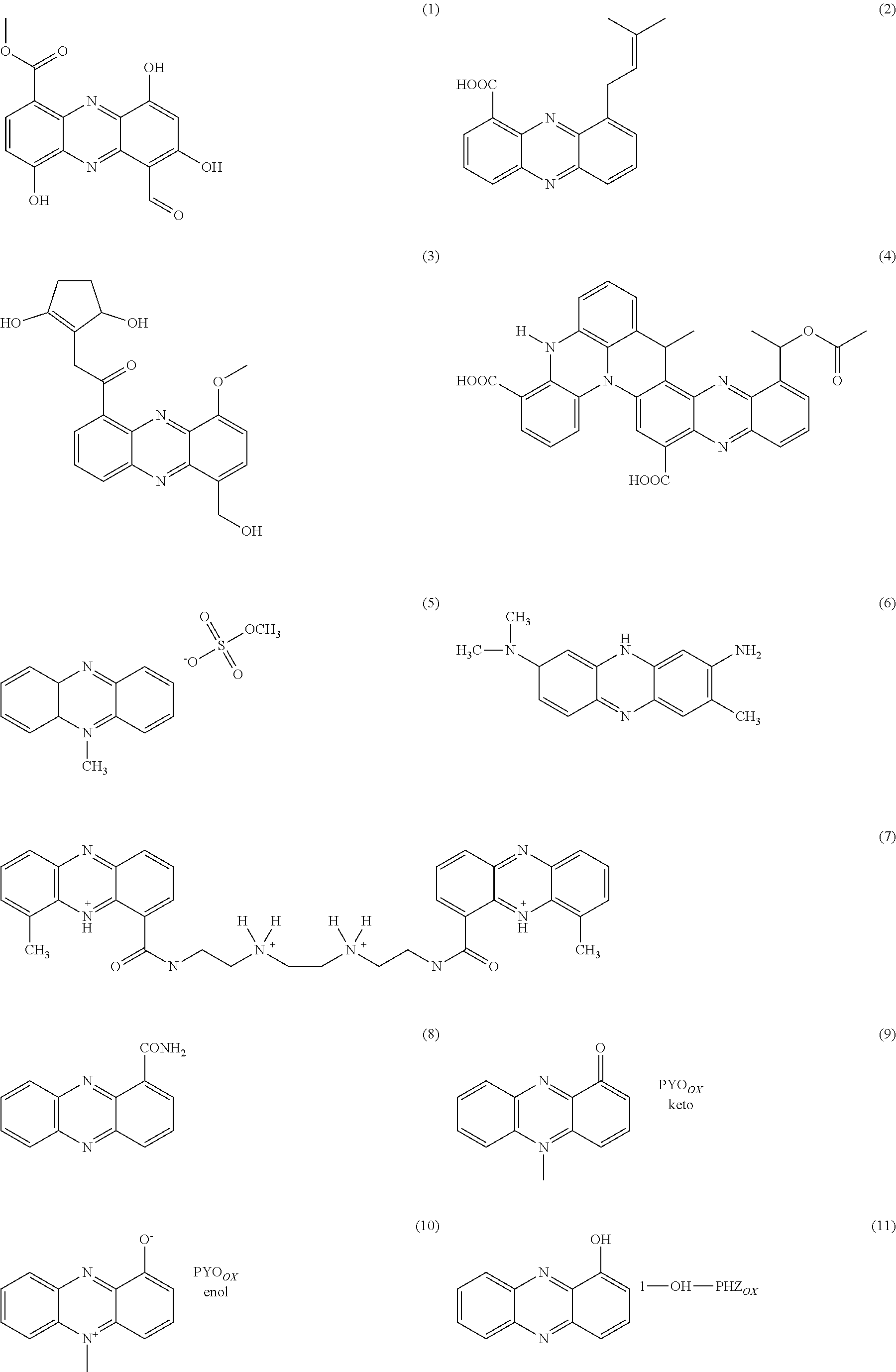

as well as additional phenazines that can be identified by a skilled person such as the exemplary phenazines described in Mentel et al. (ChemBioChem 2009, 10, 2295-2304) [3] and Pierson et al. (Appl Microbiol. Biotechnol. 2010, 86, 1659-1670) [4] and in other references cited in the instant disclosure which are incorporated herein by reference in their entirety

Phenazine pigments are mostly water soluble and are excreted into the medium. For example, pyocyanin produced by Pseudomonas aeruginosa, diffuses readily into agar-solidified media which become stained blue. Some phenazines are only sparingly water soluble and precipitate. For examples, chlororaphine, a mixture of phenazine-1-carboxamide (oxychlororaphine) and its dihydro derivative, produced by Pseudomonas chlororaphis, accumulate as isolated emerald-green crystals at the base of agar slants. Iodinin crystallizes on the surfaces of old colonies of Brevibacterium iodinum, giving them a dark-purple appearance, and phenazine-1-carboxylic acid (PCA) is deposited as golden yellow crystals in colonies of Pseudomonas aureofaciens and in the surrounding medium. It is noted, however, that the same pigment can be produced by unrelated bacteria and “achromogenic” strains of many phenazine-producers are common. A number of strains of bacteria produce more than one phenazine. It seems likely that all bacterial phenazines are derived from a common precursor.

Representative phenazines comprise pyocyanin (PYO) and Phenazine-1-carboxylic acid (PCA). Pyocyanin (PYO) is the phenazine characteristically produced by chromogenic strains of the pseudomonad, which is found as the blue pigment occasionally seen on infected wound dressings. More attention has been paid to pyocyanin than to any other phenazine. Pyocyanin is an organic base, blue in alkaline aqueous solutions but red when acidified. The differential solubility of these forms in chloroform and water was exploited for this pigment. Pyocyanin was found to be chemically reduced to a colorless form and spontaneously reoxidized in air, which has led to the discovery, the indicator and redox properties of the compound. Additionally, pyocyanin slowly decomposed to a yellow substance, no longer basic in nature, now known to be 1-hydroxyphenazine.

PCA is a yellow crystalline compound naturally produced by P. aureofaciens. The phenazine produced was readily extracted from acidified cultures with chloroform. Dilute alkali changed the color of the phenazine to orange-red and rendered it insoluble in chloroform. PCA isolated from cultures, in amounts of up to 1 g of pigment litre-1, was shown to have antibacterial activity towards a number of plant pathogens.

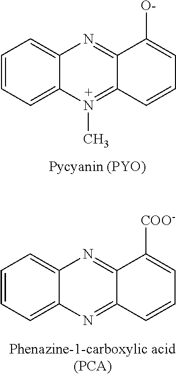

Biosynthesis as well as properties of individual phenazines are identifiable by a skilled person. In particular, phenazine natural products have been implicated in the virulence and competitive fitness of producing organisms. For example, the phenazine pyocyanin produced by Pseudomonas aeruginosa contributes to its ability to colonise the lungs of cystic fibrosis (CF) patients. Production of pyocyanin by P. aeruginosa is responsible for the bluish tint of sputum and pus associated with P. aeruginosa infections in humans. Clear correlation has been demonstrated between phenazine concentration in sputum and lung function decline. Further, phenazines are found to affect bacterial community development for P. aeruginosa.

Similarly, phenazine-1-carboxylic acid, produced by a number of Pseudomonas spp., increases survival in soil environments and has been shown to be essential for the biological control activity of certain strains. Examples are provided below for two types of phenazines known as pyocyanin and phenazine-1-carboxylic acid, respectively. For more examples of the occurrence, biochemistry and physiology of phenazine production, see Turner et al., 1986, Advances in Microbial Physiology, vol. 27, page 211-275. [5]

Phenazines targeted by phenazines degrading agents, herein described, comprise in particular pyocyanin-like phenazines which are formed by phenazines of formula (III)

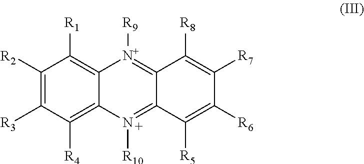

wherein R₁-R₁₀ are independently selected from hydrogen, hydroxy, alkoxy, alkyl, alkenyl, alkynyl, aryl, heteroaryl, acyl, and one of R₁-R₁₀ is a negatively charged substituent. In some of those embodiments, R₁-R₈ are independently selected from hydrogen, hydroxy, C1-C4 alkoxy, C1-C4 alkyl, C2-C4 alkenyl, C2-C4 alkynyl, and other groups identifiable to the skilled person, N+-R10 is H and R9 is CH3.

In some embodiments, pyocyanin-like phenazines comprise phenazines of formula III wherein at least one of R₁-R₈ is hydroxy group. In some embodiments, pyocyanin-like phenazines comprise phenazines of formula (III) wherein at least one of R₁-R₈ is methoxy group.

In some embodiments, pyocyanin and/or a pyocyanin-like phenazines can be represented by formula (IV)

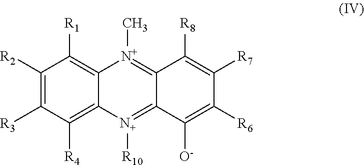

where R₁-R₄, R₆-R₈ and R₁₀ are independently selected from hydrogen, hydroxy, alkoxy, alkyl, alkenyl, alkynyl, aryl, heteroaryl, acyl, and other groups identifiable to the skilled person.

In some embodiments, pyocyanin-like phenazines comprise-methylphenazinium methyl sulfate or 3,6-diamino-10-methylacridinium or 1,8-dimethoxyphenazine or myxin, 5-methylphenazinium methyl sulfate or 3,6-diamino-10-methylacridinium or 1,8-dimethoxyphenazine or myxin and additional phenazines identifiable by a skilled person.

Embodiments here described are directed to compounds configured to be able to degrade phenazines in the sense of the disclosure, herein also defined as phenazine degrading agents

Accordingly, the term “phenazine degrading agent” as used herein indicates any compound capable of breaking down phenazine in the sense of the disclosure into smaller molecules. Phenazine degrading agents comprise proteins from bacteria capable to degrade pyocyanin to 1-OH-phenazine+formaldehyde. Additional features of degraded phenazines can be identified by a skilled person.

In some embodiments, phenazine degrading agents can be naturally produced by a bacterium capable of producing phenazines. Identification of those degrading agents can be performed, for example, by constructing a bacterial “enrichment culture” by defining a minimal growth medium where a phenazine (PCA, PYO, and additional phenazines identifiable by a skilled person) is provided as either (or both) the sole source of carbon or nitrogen. If growth is observed after many rounds of serial dilutions, phenazine-degraders can be isolated by plating the enrichment culture on an agar plate with the same medium composition. Single colonies are picked, and streaked to fresh plates, and visually checked for purity. Once pure, the 16S rDNA is sequenced and the organism can be phenotypically characterized. Other methods for identifying a bacterium capable of phenazine degradation would be identifiable to a skilled person upon reading of the present disclosure. Once a bacterium capable of degrading phenazine is identified, one or more particular enzymes responsible for phenazine degradation in the bacterium can be identified, for example, by biochemical approach and/or genetic approaches identifiable by a skilled person

In some embodiments, phenazine degrading agents herein described are synthetic phenazine degrading agents including at least one residue or chemical moiety that differs from naturally produced phenazine degrading agents.

In some embodiments, phenazine degrading agents herein described can be produced following modifications of a naturally occurring or other synthetic pyocyanin degrading agent by biochemical approach, a genetic approach and other approaches identifiable by a skilled person. In particular, a biochemical approach to provide a phenazine degrading agents herein described can comprise performing an activity assay following chemical modification of the pyocyanin demethylase, for example based on absorption or fluorescence a phenazine over time and a subsequent purifying of cell fractions to promote a disappearance of phenazine. A genetic approach to provide a phenazine degrading agents herein described can comprise employing transposition mutagenesis of the pyocyanin demethylase to make a collection of random mutants and screening them for an inability to grow on a minimal medium plus the phenazine, as described, for example, in Gallagher et al. (J. Bacteriol. 2002, 184, 6472-6480). [6] Functionality of a modified pyocyanin demethylase can be tested by a biochemical assay where the enzyme is mixed with the substrate pyocyanin. The loss of blue coloration indicates an active pyocyanin demethylase.

In some embodiments herein described, a phenazine degrading agent comprises or consists of a pyocyanin demethylase.

The term “pyocyanin demethylase” herein described refers to a type of enzymes having the ability to oxidize a methyl group of pyocyanin-like phenazines of formula (III) to formaldehyde and reduce the pyrazine ring of pyocyanin-like phenazines of formula (III) via a tautomerizing demethylation reaction. The pyocyanin demethylase uses an oxidized phenazine substrate as an electron acceptor with a methyl group to produce a reduced phenazine and formaldehyde. In general, the catalytic site of the pyocyanin demethylase contains several charged and polar residues and a nearby disulfide, also referred to as demethylating residues such as histidine, aspartate, glutamate or tyrosine, as these residues contribute to the demethylase capability of the enzyme. A derivative of a pyocyanin demethylase indicates an enzyme having a) at least 30% identity with the pyocyanin demethylase, and b) at least one demethylating residue in the catalytic site of the derivative pyocyanin demethylase, which is i) the same demethylating residue of the catalytic site of the pyocyanin demethylase or a functional equivalent thereof, and is ii) in a position equivalent to the position of the same demethylating residue of the catalytic site of the pyocyanin demethylase. The derivative of the pyocyanin demethylase to maintain a same, reduced or increased an ability to demethylate pyocyanin as will be understood by a skilled person.

An exemplary demethylation by a pyocyanin demethylase is the demethylation of PYO to 1-OH-PHZre performed by a pyocyanin demethylase herein described. The chemical reaction is shown as follows:

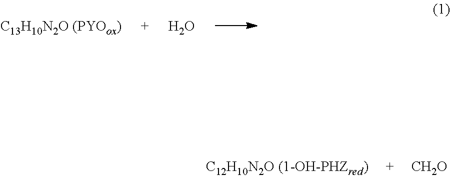

in which oxidized PYO (PYOₒₓ) and water are converted to reduced 1-OH—PHZ and formaldehyde. The reduced 1-OH—PHZ can in turn react with Fe (III) generating Fe (II) and oxidized 1-OH—PHZ (1-OH—PHZ (x). The oxidized1-OH—PHZ is also an iron chelator and can chelate Fe (III), Fe (II) as well as other metals. The generation of 1-OH—PHZ will limit the Fe concentration in pathogens such as P. aeruginosa, thus interfering with biofilm formation and/or maintenance.

In particular some embodiments, a pyocyanin demethylase in the sense of the disclosure indicates a protein from Mycobacterium fortuitum encoded by MFORT_14352 (NCBI Accession number: EJZ13467) that catalyzes pyocyanin (PYO) degradation (K. C. Costa, M. Bergkessel, S. Saunders, J. Korlach, D. K. Newman, Enzymatic degradation of phenazines can generate energy and protect sensitive organisms from toxicity. MBio 6, e01520-01515 (2015) [7] herein also referred to as PodA (pyocyanin: phenazine oxidoreductase demethylating).

In particular, a pyocyanin demethylase in the sense of the disclosure can have sequence MTGKTKPAIIGGVAITALAAAGLGVWLFTDGRGGRSTTEPVTMTLDVKNDQVAKHDFG KPGMDVGDMDIFSDILSVDGKQVGYDGGACFFTNVTPDNPMTYCELTIHLDAGEIFARS LTPHTLAPFTMAITGGTGEYANSKGELTVSGVATPDEKYELKLTK (SEQ ID NO: 1) herein also indicated as PodA unless otherwise indicated.

In some embodiments, a pyocyanin demethylase according to the present disclosure is an engineered pyocyanin demethylases PodA having sequence

(SEQ ID NO: 1)
M₁T₂G₃K₄T₅K₆P₇A₈I₉I₁₀G₁₁G₁₂V₁₃A₁₄I₁₅T₁₆A₁₇
L₁₈A₁₉A₂₀A₂₁G₂₂L₂₃G₂₄V₂₅W₂₆L₂₇F₂₈T₂₉D₃₀G₃₁
R₃₂G₃₃G₃₄R₃₅S₃₆T₃₇T₃₈E₃₉P₄₀V₄₁T₄₂M₄₃T₄₄L₄₅
D₄₆V₄₇K₄₈N₄₉D₅₀Q₅₁V₅₂A₅₃K₅₄H₅₅D₅₆F₅₇G₅₈K₅₉
P₆₀G₆₁M₆₂D₆₃V₆₄G₆₅D₆₆M₆₇D₆₈I₆₉F₇₀S₇₁D₇₂I₇₃
L₇₄S₇₅V₇₆D₇₇G₇₈K₇₉Q₈₀V₈₁G₈₂Y₈₃D₈₄G₈₅G₈₆A₈₇
C₈₈F₈₉F₉₀T₉₁N₉₂V₉₃T₉₄P₉₅D₉₆N₉₇P₉₈M₉₉T₁₀₀
Y₁₀₁C₁₀₂E₁₀₃L₁₀₄T₁₀₅I₁₀₆H₁₀₇L₁₀₈D₁₀₉A₁₁₀
G₁₁₁E₁₁₂I₁₁₃F₁₁₄A₁₁₅R₁₁₆S₁₁₇L₁₁ST₁₁₉P₁₂₀
H₁₂₁T₁₂₂L₁₂₃A₁₂₄P₁₂₅F₁₂₆T₁₂₇M₁₂₈A₁₂₉I₁₃₀
T₁₃₁G₁₃₂G₁₃₃T₁₃₄G₁₃₅E₁₃₆Y₁₃₇A₁₃₈N₁₃₉S₁₄₀
K₁₄₁G₁₄₂E₁₄₃L₁₄₄T₁₄₅V₁₄₆S₁₄₇G₁₄₈V₁₄₉A₁₅₀
T₁₅₁P₁₅₂D₁₅₃E₁₅₄K₁₅₅Y₁₅₆E₁₅₇L₁₅₈K₁₅₉L₁₆₀
T₁₆₁K₁₆₂,

having replacement in positions A53, 173, A87, T91, M99, A129 and K141 to increase stability, yield and/or activity of the pyocyanin demethylase in SEQ ID NO: 1 or portions thereof.

In particular the pyocyanin demethylase further modified to include at least two replacements in SEQ ID NO: 1 selected from
A53 with L, N, or V
173 with I, E, K, L, Q, R, T or V
A87 with C, I, T or V
T91 with V
M99 with M, C, F, I, K, R, V or Y
A129 with A, C, S, T, or V
K141 with K, S or T
   and configured to be capable of the engineered pyocyanin demethylase or a derivative thereof to demethylate pyocyanin and/or a pyocyanin-like phenazines of formula (III)

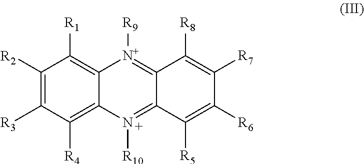

where R₁-R₁₀ are independently selected from hydrogen, hydroxy, alkoxy, alkyl, alkenyl, alkynyl, aryl, heteroaryl, acyl, and other groups identifiable to the skilled person, and one of R₁-R₁₀ is a negatively charged substituent (formal charge of

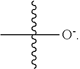

In preferred embodiments, the engineered demethylase in the sense of the present disclosure has replacements in positions 73, 87, 91, 99 and 129, or in positions 73, 87, 99, 129 and 141, or in position 73, 87, 91, 129 and 141, or in positions 53, 73, 87, 99 and 129, as mutations in those residues are expected to result in an increased yield with respect to wild type PodA of SEQ ID NO 1 (see example section reporting data for representative example of SEQ ID NO:3)

In more preferred embodiments, the engineered demethylase in the sense of the disclosure has at least two of a replacement in position 73 selected from I73T, I73K, I73L, I73R, a replacement in position 87 selected from A87V, and A87I, a replacement in position 99 selected from M99V and M99T, a replacement in position 129 selected from A129V and A129T, the replacement K141T, in position 141, the replacement T91V, in position 91 and the replacement A53N, in position 53 of SEQ ID NO: 1.

In additional preferred embodiments, an engineered demethylase in the sense of the disclosure is PodA2 of SEQ ID NO: 1, herein also PodA2 unless otherwise indicated, having replacements I73T, A87V, T91V, M99V and A129V, PodA3 of SEQ ID NO: 1, herein also PodA3 unless otherwise indicated, having replacements I73K, A87V, T91V, M99V, A129V, PodA5 of SEQ ID NO: 1, herein also PodA5 unless otherwise indicated, having replacements I73L, A87I, M99V, A129V, K141T, PodA6 of SEQ ID NO: 1, herein also PodA6 unless otherwise indicated, having replacements I73R, A87V, T91V, M99V, A129T, PodA7 of SEQ ID NO: 1, herein also PodA7 unless otherwise indicated, having replacements I73L, A87I, T91V, M99T, and A129V, PodA8 of SEQ ID NO: 1, herein also PodA8 unless otherwise indicated, having replacements A53N, I73R, A87V. T91V. A129V, PodA9 of SEQ ID NO: 1, herein also PodA9 unless otherwise indicated, having replacements I73K, A87V. T91V, A129V and K141T, and/or PodA10 of SEQ ID NO: 1, herein also PodA 10 unless otherwise indicated, having replacements A53N, I73T, A87V, M99V, A129T.

In some embodiments, phenazine degrading agents encompass derivatives of pyocyanin demethylase in the sense of the disclosure and in particular of engineered pyocyanin demethylase in the sense of the disclosure.

A derivative of a pyocyanin demethylase in the sense of the disclosure indicates a variant of a protein that has at least 30% identity with the reference sequence while retaining the ability to demethylate pyocyanin.

The term “percent identity” refers to a quantitative measurement of the similarity between sequences of a polypeptide or a polynucleotide and, in particular, indicates the amount of characters that match between two different sequences. Commonly used similarity searching programs, like BLAST, PSI-BLAST (Altschul S F, M. T., Schäffer A A, Zhang J, Zhang Z, Miller W, Lipman D J., Gapped BLAST and PSI-BLAST: a new generation of protein database search programs. Nucleic Acids Res., 1997. 25 (17): p. 14), SSEARCH (Smith T F, W. M., Identification of common molecular subsequences. J Mol Biol, 1981. 147 (1): p. 3, WR. P., Searching protein sequence libraries: comparison of the sensitivity and selectivity of the Smith-Waterman and FASTA algorithms. Genomics, 1991. 11 (3): p. 16) FASTA (Pearson W R, L.D., Improved tools for biological sequence comparison. Proc Natl Acad Sci USA, 1988. 85 (8): p. 5) [11] and the HMMER3 9 (Johnson L S, E.S., Portugaly E, Hidden Markov model speed heuristic and iterative HMM search procedure. BMC Bioinformatics, 2010. 11 (431): p. 8.) [12] can produce accurate statistical estimates, ensuring that protein sequences that share significant similarity also have similar structures.

The similarity between sequences is typically measured by a process that comprises the steps of aligning the two polypeptide or polynucleotide sequences to form aligned sequences, then detecting the number of matched characters, i.e. characters similar or identical between the two aligned sequences and calculating the total number of matched characters divided by the total number of aligned characters in each polypeptide or polynucleotide sequence, including gaps. The similarity result is expressed as a percentage of identity.

For example in embodiment wherein the pyocyanin demethylase is, PodA derivatives in the sense of the disclosure encompass homologous proteins of PodA with at least 30% identity with PodA of SEQ ID NO: 1 and that possesses the demethylating residues at positions equivalent to D72, E154, and Y156 in PodA.

In some embodiments, a derivative of a pyocyanin demethylase comprise a truncated version the protein encoded by MFORT_14352 (lacking a predicted N-terminal, membrane-spanning helix), hereafter referred to as PodA₃₀₋₁₆₂. The gene expressing PodA₃₀₋₁₆₂ derived from Mycobacterium was heterologously expressed in E. coli. from Escherichia coli. In particular, PodA₃₀₋₁₆₂ in the sense of the disclosure can have sequence MDGRGGRSTTEPVTMTLDVKNDQVAKHDFGKPGMDVGDMDIFSDILSVDGKQVGYD GGACFFTNVTPDNPMTYCELTIHLDAGEIFARSLTPHTLAPFTMAITGGTGEYANSKGEL TVSGVATPDEKYELKLTKAENLYFQ (SEQ ID NO: 2) as described in U.S. Pat. No. 10,913,936 filed on Mar. 22, 2017 and issued on Feb. 9, 2021, herein incorporated by reference in its entirety.

In some embodiments, a derivative of a pyocyanin demethylase comprise a truncated version the protein encoded by MFORT_14352 (lacking a predicted N-terminal, membrane-spanning helix), hereafter referred to as PodA₄₁₋₁₆₂. The gene expressing PodA₄₁₋₁₆₂ derived from Mycobacterium was heterologously expressed in E. coli. from Escherichia coli. In particular, PodA₄₁₋₁₆₂ in sense of the the disclosure can have sequence VTMTLDVKNDQVAKHDFGKPGMDVGDMDIFSDILSVDGKQVGYDGGACFFTNVTPDN PMTYCELTIHLDAGEIFARSLTPHTLAPFTMAITGGTGEYANSKGELTVSGVATPDEKYE LKLTK (SEQ ID NO: 3).

A table indicating the correspondence of the positions of residues A53, 173, A87, M99, A129 and K141 of PodA of SEQ ID NO: 1 in the derivative PodA₃₀₋₁₆₂ of SEQ ID NO: 2 and in derivative PodA₄₁₋₁₆₂ of SEQ ID NO: 3 is reported in Table 1 below

TABLE 1
Overview PodA residues/positions
for stability, yield and/or activity

| PodA-SEQ | PodA₃₀₋₁₆₂- | PodA₄₁₋₁₆₂- |
|---|---|---|
| ID NO: 1 | SEQ ID NO: 2 | SEQ ID NO: 3 |
| A53 | A24 | A13 |
| I73 | I44 | I33 |
| A87 | A58 | A47 |
| T91 | T62 | T51 |
| M99 | M70 | M59 |
| A129 | A100 | A89 |
| K141 | K112 | K101 |

Whenever not accompanied by a specific indication of a SEQ ID NO the above residues and position replaceable to increase stability, yield and/or activity of pyocyanin demethylase PodA or a derivative thereof are indicated with respect to the PodA sequence (SEQ ID NO: 1) as customary in the field and would be understood by a skilled person.

A skilled person will also be able to identify the residues and positions reported in Table 1 in sequences of derivatives of pyocyanin demethylases upon review of the present disclosure.

Accordingly, in some embodiments, an engineered pyocyanin demethylase PodA₃₀₋₁₆₂ or a derivative thereof are described, the pyocyanin demethylase comprising sequence

(SEQ ID NO: 2)
D₁G₂R₃G₄G₅R₆S₇T₈T₉E₁₀P₁₁V₁₂T₁₃MI₄TI₅LI₆D₁₇
V₁₈K₁₄N₂₀D₂IQ₂₂V₂₃A₂₄K₂₅H₂₆D₂₇F₂₈G₂₉K₃₀P₃₁
G₃₂M₃₃D₃₄V₃₅G₃₆D₃₇M₃₈D₃₉I₄₀F₄₁S₄₂D₄₃I₄₄L₄₅
S₄₆V₄₇D₄₈G₄₉K₅₀Q₅₁A₅₂G₅₃Y₅₄D₅₅G₅₆G₅₇A₅₈C₅₉
F₆₀F₆₁T₆₂N₆₃V₆₄T₆₅P₆₆D₆₇N₆SP₆₉M₇₀T₇₁Y₇₂C₇₃
E₇₄L₇₅T₇₆I₇₇H₇₈L₇₉D₈₀A₈₁G₈₂E₈₃I₈₄F₈₅A₈₆R₈₇
S₈₈L₈₉T₉₀P₉₁H₉₂T₉₃L₉₄A₉₅P₉₆F₉₇T₉₈M₉₉A₁₀₀
I₁₀₁T₁₀₂G₁₀₃G₁₀₄T₁₀₅G₁O₆E₁₀₇Y₁₀₈AI₀₉N₁₁₀
S₁₁₁K₁₁₂G₁₁₃E₁₁₄L₁₁₅T₁₁₆V₁₁₇S₁₁₈G₁₁₉V₁₂₀
A₁₂₁T₁₂₂P₁₂₃D₁₂₄E₁₂₅K₁₂₆Y₁₂₇E₁₂₈L₁₂₉K₁₃₀
L₁₃₁T₁₃₂K₁₃₃A₁₃₄E₁₃₅N₁₃₆L₁₃₇Y₁₃₈F₁₃₉Q₁₄₀,

the pyocyanin demethylase further modified to include at least two replacements in SEQ ID NO: 2 selected from
A24 with L, N, or V
I44 with I, E, K, L, Q, R, T or V
A58 with C, I, T or V
T62 with V
M70 with M, C, F, I, K, R, V or Y
A100 with A, C, S, T, or V
K112 with K, S or T
   and configured to be capable of the engineered pyocyanin demethylase or a derivative thereof to demethylate pyocyanin and/or a pyocyanin-like phenazines of formula (III)

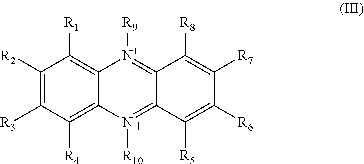

where R₁-R₁₀ are independently selected from hydrogen, hydroxy, alkoxy, alkyl, alkenyl, alkynyl, aryl, heteroaryl, acyl, and other groups identifiable to the skilled person, and one of Ri-R₁₀ is a negatively charged substituent (formal charge of −1) such as

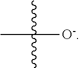

In some preferred embodiments, and engineered pyocyanin demethylase PodA₃₀₋₁₆₂ has replacements in positions 44, 58, 62, 70 and 100, or in positions 44, 58, 70, 100 and 112, or in position 44, 58, 62, 100 and 112, or in positions 24, 44, 58, 70 and 100 of SEQ ID NO: 2, as mutations in those residues are expected to result in an increased yield with respect to wild type PodA (see example section reporting data in connection with representative example of SEQ ID NO: 3).

In more preferred embodiments, the engineered demethylase PodA₃₀₋₁₆₂ has at least two of a replacement in position 44 selected from 144T, I44K, I44L, I44R, a replacement in position 58 selected from A58 V, and A58 I, a replacement in position 70 selected from M70V and M70T, a replacement in position 100 selected from A100V and A100T, the replacement K112T, in position 112, the replacement T62V, in position 62 and the replacement A24N, in position 24 of SEQ ID NO: 2.

In additional preferred embodiments, the engineered demethylase in the sense of the disclosure is PodA2 of SEQ ID NO: 2, herein also PodA2₃₀-162 unless otherwise indicated, having replacements 144T, A58V, T62V, M70V and A100V, PodA3 of SEQ ID NO: 2, herein also PodA3₃₀₋₁₆₂ unless otherwise indicated, having replacements I44K, A58, T62V, M70V, A100V, PodA5 of SEQ ID NO: 2, herein also PodA5₃₀₋₁₆₂ unless otherwise indicated, having replacements I44L, A58I, M70V, A100V, K112T, PodA6 of SEQ ID NO: 2, herein also PodA630-162 unless otherwise indicated, having replacements I44R, A58V, T62V, M70V, A100T, PodA7 of SEQ ID NO: 2, herein also PodA7₃₀-162 unless otherwise indicated, having replacements I44L, A58I, T62V, M70T, and A100V, PodA8 of SEQ ID NO: 2, herein also PodA830-162 unless otherwise indicated, having replacements, A24N, I44R, A58V, T62V, A100V, PodA9 of SEQ ID NO: 2, herein also PodA930-162 unless otherwise indicated, having replacements I44K, A58V, T62V, A100V and K112T, and/or PodA10 of SEQ ID NO: 2, herein also PodA2₃₀-162 unless otherwise indicated, having replacements A24N, 144T, A58V, M70V, A100T.

In some embodiments, an engineered pyocyanin demethylase can be PodA₄₁₋₁₆₂ or a derivative thereof, the pyocyanin demethylase comprising sequence

(SEQ ID NO: 3)
V₁T₂M₃T₄L₅D₆V₇K₈N₉D₁₀Q₁₁V₁₂A₁₃K₁₄H₁₅D₁₆F₁₇
G₁₈K₁₉P₂₀G₂₁M₂₂D₂₃V₂₄G₂₅D₂₆M₂₇D₂₈I₂₉F₃₀S₃₁
D₃₂I₃₃L₃₄S₃₅V₃₆D₃₇G₃₈K₃₉Q₄₀V₄₁G₄₂Y₄₃D₄₄G₄₅
G₄₆A₄₇C₄₈F₄₉F₅₀T₅₁N₅₂V₅₃T₅₄P₅₅D₅₆N₅₇P₅₈M₅₉
T₆₀Y₆₁C₆₂E₆₃L₆₄T₆₅I₆₆H₆₇L₆₈D₆₉A₇₀G₇₁E₇₂I₇₃
F₇₄A₇₅R₇₆S₇₇L₇₈T₇₉P₈₀H₈₁T₈₂L₈₃A₈₄P₈₅F₈₆T₈₇
M₈₈A₈₉I₉₀T₉₁G₉₂G₉₃T₉₄G₉₅E₉₆Y₉₇A₉₈N₉₉S₁₀₀
K₁₀₁G₁₀₂E₁₀₃L₁₀₄T₁₀₅V₁₀₆S₁₀₇G₁₀₈V₁₀₉A₁₁₀
T₁₁₁P₁₁₂D₁₁₃E₁₁₄K₁₁₅Y₁₆E₁₁₇L₁₁₈K₁₁₉L₁₂₀
T₁₂₁K₁₂₂,

the pyocyanin demethylase further modified to include at least two replacements in SEQ ID NO: 3 selected from
A13 with L, N, or V
I33 with I, E, K, L, Q, R, Tor V
A47 with C, I, T or V
T51 with V
M59 with M, C, F, I, K, R, V or Y
A89 with A, C, S, T, or V
K101 with K, S or T
   and configured to be capable of the engineered pyocyanin demethylase or a derivative thereof to demethylate pyocyanin and/or a pyocyanin-like phenazines of formula (III)

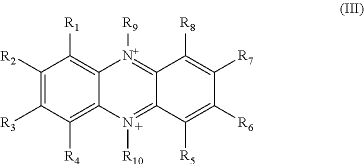

where R₁-R₁₀ are independently selected from hydrogen, hydroxy, alkoxy, alkyl, alkenyl, alkynyl, aryl, heteroaryl, acyl, and other groups identifiable to the skilled person, and one of R₁-R₁₀ is a negatively charged substituent (formal charge of −1) such as

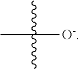

In preferred embodiments, the engineered demethylase PodA₄₁₋₁₆₂ has replacements in positions 33, 47, 51, 59 and 89, or in positions 33, 47, 59, 89 and 101, or in position 33, 47, 51, 89 and 101, or in positions 13, 33, 47, 59 and 89, as mutations in those residues are expected to result in an increased yield with respect to PodA₄₁₋₁₆₂ of SEQ ID NO 3 (see example section)

In more preferred embodiments, the engineered demethylase PodA₄₁₋₁₆₂ has at least two of a replacement in position 33 selected from 133T, I33K, I33L, I33R, a replacement in position 47 selected from A47V, and A47I, a replacement in position 59 selected from M59V and M59T, a replacement in position 89 selected from A89V and A89T, the replacement K101T, in position 101, the replacement T51V, in position 51 and the replacement A13N, in position 13 of SEQ ID NO: 3.

In additional preferred embodiments, an engineered demethylase in the sense of the disclosure is PodA2 of SEQ ID NO: 3, herein also PodA2₄₁₋₁₆₂ unless otherwise indicated, having replacements 133T, A47V, T51V, M59V and A89V, PodA3 of SEQ ID NO: 3, herein also PodA3₄₁₋₁₆₂ unless otherwise indicated, having replacements I33K, A47V, T51V, M59V, A89V, PodA5 of SEQ ID NO: 3, herein also PodA5₄₁₋₁₆₂ unless otherwise indicated, having replacements I33L, A47I, M59V, A89V, K101T, PodA6 of SEQ ID NO: 3, herein also PodA641-162 unless otherwise indicated having replacements I33R, A47V, T51V, M59V, A89T, PodA7 of SEQ ID NO: 3, herein also PodA7₄₁₋₁₆₂ unless otherwise indicated, having replacements I33L, A47I, T51V, M59T, and A89V, PodA8 of SEQ ID NO: 3, herein also PodA841-162 unless otherwise indicated, having replacements, A13N, I33R, A47V, T51V. A89V, PodA9 of SEQ ID NO: 3, herein also PodA941-162 unless otherwise indicated, having replacements I33K, A47V, T51V. A89V and K101T, and/or PodA10 of SEQ ID NO: 3, herein also PodA1041-162 unless otherwise indicated, having replacements A13N, 133T, A47V. M59V, A89T.

In some embodiments, pyocyanin demethylase herein described can include additional sequences at the N-terminus or C-terminus, e.g. typically methionine for the start codon, as will be understood by a skilled person.

In particular, in some embodiments, reported in the Example section PodA 1041-162 can have sequence MVTMTLDVKNDQVNKHDFGKPGMDVGDMDIFSDTLSVDGKQVGYDGGVCFFTNVTP DNPVTYCELTIHLDAGEIFARSLTPATLAPFTMTITGGTGEYANSKGELTVSGVATPDEK YELKLTK (SEQ ID NO: 4) as will be understood by a skilled person.

In some of those embodiments, a PodA derivative can have a 40% identity, a 50% identity or a 60% identity with PodA of SEQ ID NO:1, SEQ ID NO: 2, SEQ ID NO: 3 or SEQ ID NO: 4. In some embodiments, a PodA derivative can have 70% or higher identity with PodA of SEQ ID NO:1 SEQ ID NO: 2, SEQ ID NO: 3 or SEQ ID NO: 4.

In pyocyanin demethylases and derivatives herein described, the demethylating residues can be identified by first aligning a PodA derivative to SEQ ID NO:1 SEQ ID NO: 2, SEQ ID NO: 3 or SEQ ID NO: 4 and then identifying the residues in the PodA derivative that correspond to the demethylating residues of SEQ ID NO:1, SEQ ID NO: 2, SEQ ID NO: 3 or SEQ ID NO: 4 in the aligned columns as would be understood to a person skilled in the art.

The PodA derivatives in some embodiments encompass homologous proteins of PodA with at least 30% identity, 40% identity, 50% identity or >70% identity with PodA of SEQ ID NO: 1, SEQ ID NO: 2, SEQ ID NO: 3 or SEQ ID NO: 4 while retaining the ability to demethylate pyocyanin.

In some embodiments, derivatives of pyocyanin demethylase comprise homologous proteins of PodA having SEQ ID NO: 1, SEQ ID NO: 2, SEQ ID NO: 3 or SEQ ID NO: 4, with at least 30% identity, in which one or more demethylating residues within the enzyme's catalytic site. such as H121, F70, D68, D72, E154 and Y156 of SEQ ID NO: 1 or corresponding residues in SEQ ID NO: 2, SEQ ID NO: 3 or SEQ ID NO: 4, are replaced with a functionally equivalent residue.

In some embodiments, derivatives of the engineered pyocyanin demethylase comprise homologous proteins of PodA having SEQ ID NO: 1, SEQ ID NO: 2. SEQ ID NO: 3 or SEQ ID NO: 4 in which at least 30% identity, in which one or more demethylating residues within the enzyme's catalytic site, such as H121, F70, D68, D72, E154 and Y156 of SEQ ID NO: 1 or corresponding residues in SEQ ID NO:2, SEQ ID NO:3 and SEQ ID NO:4, are replaced with a functionally equivalent residue.

A functionally equivalent residue of an amino acid used herein typically refers to other amino acid residues having physiochemical and stereochemical characteristics substantially similar to the original amino acid. The physiochemical characteristics include water solubility (hydrophobicity or hydrophilicity), dielectric and electrochemical properties, physiological pH, partial charge of side chains (positive, negative or neutral) and other properties identifiable to a person skilled in the art. The stereochemical characteristics include spatial and conformational arrangement of the amino acids and their chirality. For example, glutamic acid is considered to be a functionally equivalent residue to aspartic acid in the sense of the current disclosure. Tyrosine and tryptophan are considered as functionally equivalent residues to phenylalanine. Arginine and lysine are considered as functionally equivalent residues to histidine.

In particular, the demethylating residue H121 of PodA can be replaced by a protonating residue such as arginine or lysine: D68, D72, E154 or Y156 can be replaced with a negatively charged residue such as aspartate, glutamate or tyrosine; F70 can be replaced by an aromatic residue such as tyrosine or tryptophan.

In some embodiments, a derivative pyocyanin demethylase herein described can be provided using directed evolution from a pyocyanin demethylase or a derivative thereof herein described.

In some embodiments, a pyocyanin demethylase derivative can be provided by protein engineering methods identifiable by those skilled in the art, such as methods based on rational design of modified pyocyanin demethylase derivatives and/or directed evolution techniques. The term “rational design” indicates a process wherein detailed knowledge of the structure and function of a protein is used to make desired changes, employing site-directed mutagenesis and other methods known to those skilled in the art.

In one exemplary embodiment, a derivative pyocyanin demethylase can be designed and generated using computational strategies by introducing mutations to PodA having SEQ ID: NO: 1 SEQ ID NO: 2, SEQ ID NO: 3 or SEQ ID NO: 4. The designed pyocyanin demethylase derivatives can potentially possess enhanced stability, higher yield, and comparable or even enhanced catalytic efficiency compared to that of PodA having SEQ ID: NO: 1 SEQ ID NO: 2, SEQ ID NO: 3 or SEQ ID NO: 4. Many computational rational design tools can be used for performing such task. For example, automated algorithm based on atomistic Rosetta modeling and phylogenetic sequence information as described in Goldenzweig et. al, Molecular Cell 63, 337-346, 2016 can be used to computationally scan various pyocyanin demethylase variants.

In another exemplary embodiment, the genetic sequence corresponding to the pyocyanin demethylase can be mutated using error-prone PCR or another technique identifiable to the skilled person to produce a library of mutated genetic sequences. The proteins expressed by the mutant sequences can be screened for phenazine degrading activity against specific or broad ranges of phenazines, for example, by the spectrophotometric measurement of phenazine levels over time. The proteins thus identified to be able to degrade a specific phenazine or broad range of phenazines can be synthesized, for example, in a bacterium using recombinant DNA techniques known to the skilled person. The term “directed evolution” indicates a process wherein random mutagenesis is applied to a protein, and a selection regime is used to pick out variants that have one or more desired properties, such as selecting variants with pyocyanin demethylase activity. Directed evolution requires no prior structural knowledge of a protein, nor is it necessary to be able to predict what effect a given mutation will have. Accordingly, the sequence and structure of known pyocyanin demethylases can be modified using protein engineering techniques to provide new pyocyanin demethylase variants.

In some embodiments, a pyocyanin demethylase or a derivative thereof or other phenazine degrading agents herein described, can be obtained from gene expression of an encoding polynucleotide. Polynucleotides encoding pyocyanin demethylase derivatives can be cloned using commercially available reagents from vendors such as Qiagen, Invitrogen, Applied Biosystems, Promega, and others, following standard molecular biology methods known in the art, such as those described in Sambrook and Russell (2001) Molecular Cloning, A Laboratory Manual.

Synthetic DNA. Genomic DNA or cDNA encoding pyocyanin demethylase derivatives can be cloned into an expression vector. Expression vectors can comprise plasmid DNA, viral vectors, or non-viral vectors, among others known to those skilled in the art, comprising appropriate regulatory elements such as promoters, enhancers, and post-transcriptional and post-translational regulatory sequences, as would be understood by a skilled person. Promoters can be constitutively active or inducible. RNA can be isolated from a cell, such as Mycobacterium fortuitum and cDNA produced by reverse transcription using standard techniques and commercial kits. Alternatively, genomic DNA can be purified from the cell, and cDNA or genomic DNA encoding one or more pyocyanin demethylases isolated, following methods known to those in the art. PCR-based amplification of the gene of interest can be performed using appropriately designed primer pairs (e.g. using PrimerDesign or other programs known to those skilled in the art). An encoded tag can be incorporated into the primer design (e.g. encoding a His-tag designed to be fused to the N- or C-terminus of the recombinant enzyme) to facilitate protein purification (e.g. using commercially-available His-tagged protein purification columns/kits), as described below. PCR-based amplification can be followed by ligation (e.g. using T4 DNA ligase) of the amplicon into an appropriate expression cassette in a plasmid suitable for propagation in bacteria or other cells, such as transformation-competent E. coli, followed by growth of transformed cell cultures, purification of the plasmid for confirmation of the cloned pyocyanin demethylase by DNA sequence analysis, among other methods known to those skilled in the art.

Cloned recombinant pyocyanin demethylases can be expressed using cell-based methods, or cell-free methods, following standard techniques and using commercially available kits. Cell-based methods for expression of recombinant enzymes can include expression in prokaryotic or eukaryotic cell cultures, such as E. coli or other bacterial cells, yeast strains, insect cells, or mammalian cells, among others known to those skilled in the art.

In some embodiments, the pyocyanin demethylase derivatives in the sense of the disclosure encompass a PodA having a SEQ ID NO: 1 SEQ ID NO: 2, SEQ ID NO: 3 or SEQ ID NO: 4, an engineered pyocyanin demethylase or a derivative thereof, further linked to one or more other proteins, polypeptides, or domains to form a recombinant fusion protein.

Recombinant fusion proteins can be created artificially using recombinant DNA technology identifiable by a person skilled in the art of molecular biology. In general, the methods for producing recombinant fusion proteins comprise removing the stop codon from a cDNA or genomic sequence coding for the PodA protein having a SEQ ID NO: 1 SEQ ID NO: 2, SEQ ID NO: 3 or SEQ ID NO: 4 or a derivative thereof, then appending the cDNA or genomic sequence of the second protein in frame through ligation or overlap extension PCR. Optionally, PCR primers can further encode a linker of one or more amino acids residues and/or a PCR primer-encoded protease cleavage site placed between two proteins, polypeptides, or domains or parts thereof. The resulting DNA sequence will then be expressed by a cell or other protein expression system as a single protein. A fusion protein can also comprise a linker of one or more amino acids residues, which can enable the proteins to fold independently and retain functions of the original separate proteins or polypeptides or domains or parts thereof. Linkers in protein or peptide fusions can be engineered with protease cleavage sites that can enable the separation of one or more proteins, polypeptides, domains or parts thereof from the rest of the fusion protein.

In other embodiments, a pyocyanin demethylase fusion protein can be generated using directed evolution approaches such as DNA shuffling and others known to those skilled in the art.

In some embodiments, a pyocyanin demethylase or a derivative thereof can further comprise at least one tag having the ability to tag the pyocyanin demethylase, without affecting the enzyme's ability to degrade phenazine. The tag allows the pyocyanin demethylase to bind to tag-specific antibodies, or other molecules, depending on the tag selection.

The term “tag” as used herein indicates peptide sequences genetically grafted onto a recombinant protein. Tags can be removed by chemical agents or by enzymatic means, such as proteolysis or splicing, as known to a skilled person in the art. Tags can be attached to proteins for various purposes. For example, affinity tags can be appended to proteins so that the tagged protein can be purified from a crude biological source using an affinity technique. Exemplary affinity tags include poly-Histidine (His tag), chitin binding protein (CBP), maltose binding protein (MBP), and glutathione-S-transferase (GST), among others known to those skilled in the art. The poly(His) tag is a commonly used protein tag which binds to metal matrices. Solubilization tags can be used to assist in the proper folding in proteins and keep them from precipitating. Exemplary solubilization tags include thioredoxin (TRX) and poly (NANP). Some affinity tags have a dual role as a solubilization agent, such as MBP, and GST. Chromatography tags can be used to alter chromatographic properties of the protein to afford different resolution across a particular separation technique. Other tags applicable to the current disclosure would be identifiable to a person of ordinary skill in the art.

In some embodiments, a pyocyanin demethylase, an engineered pyocyanin demethylase or a derivative thereof can comprise a His-tag or other tags herein described attached using standard molecular biology techniques known to those skilled in the art. For example, a tag can be added by inserting the polynucleotide encoding a protein of interest in a vector that has the tag configured to fuse at the N-terminus or C-terminus. The tag can also be added using primers encoding the tag then fused to the N-terminus or C-terminus of the gene by PCR. Alternatively or in addition, methods such as overlap extension PCR and infusion HD cloning can be used to insert the tag at a site between the N-terminus and C-terminus of the protein-coding polynucleotide. Optionally, PCR primers can further encode a linker of one or more amino acids residues placed between the protein of interest and the tag to prevent the tag from affecting the activity of the protein being tagged and/or a PCR primer-encoded protease cleavage site. The choice of the location where a tag is inserted to a protein sequence depends mainly on the structural and functional features of the protein and the intended downstream methods employing the use of the tag, as would be understood to a person skilled in the art.

In some embodiments, a tagged pyocyanin demethylase, or derivative thereof is PodA of SEQ ID NO: 1 or PodA₃₀₋₁₆₂ of SEQ ID NO: 2, or PodA₄₁₋₁₆₂ of SEQ ID NO: 3, or PodA10 of SEQ ID NO: 4, further comprising a tag such as a His tag. For example, PodA₄₁₋₁₆₂ comprising a C-terminal His tag and a TEV protease cleavage site can facilitate nickel affinity purification of PodA and/or derivatives thereof. Affinity-purified PodA and/or derivatives thereof can be generated by expression in an expression system such as E. coli under control of an expression vector, followed by nickel affinity purification followed by cleavage of the 6x-His tag by TEV protease.

Additional phenazine degrading agents and engineered pyocyanin demethylase in the sense of the disclosure can be identified with methods herein described. The method comprises contacting a candidate demethylase protein with a pyocyanin-like phenazine and detecting the ability of the candidate agent to inactivate said pyocyanin-like phenazine and/or a phenazine related pathway in the bacteria. The system comprises one or more phenazine producing bacteria able to produce pyocyanin-like phenazine, and one or more agents capable of detecting phenazine and/or phenazine related pathways. In some embodiments of the methods and systems, the bacteria comprise persister cells.

The terms “detect” or “detection” as used herein indicates the determination of the existence, presence, or fact of a target in a limited portion of space, including but not limited to a sample, a reaction mixture, a molecular complex and a substrate. The “detect” or “detection” as used herein can comprise determination of chemical and/or biological properties of the target, including but not limited to ability to interact, and in particular bind, other compounds, ability to activate another compound and additional properties identifiable by a skilled person upon reading of the present disclosure. The detection can be quantitative or qualitative. A detection is “quantitative” when it refers, relates to, or involves the measurement of quantity or amount of the target or signal (also referred to as quantitation), which includes but is not limited to any analysis designed to determine the amounts or proportions of the target or signal. A detection is “qualitative” when it refers, relates to, or involves identification of a quality or kind of the target or signal in terms of relative abundance to another target or signal, which is not quantified.

In some embodiments, activity of phenazine degrading agents herein described can be detected by detecting difference in the amount of phenazine in bacteria before and after administration of the phenazine degrading agents herein described. The amount of phenazine in the bacteria before and after reduction of the quantity of phenazine according to the methods described herein can be measured by methods identifiable to a skilled person upon reading of the present disclosure. For example, the quantity of phenazine in bacterial culture before and after reduction of the quantity of phenazine can be measured by directly loading the filtrate of the culture onto a HPLC column and analyzing the filtrate as done by Dietrich et al. (Molecular Microbiology 2006, 61, 1308-1321). Additional quantification techniques can be identified by a skilled person and can include, for example, using time-lapsed spectral multiphoton fluorescence microscopy of Sullivan et al., (ACS Chemical Biology 2011, 6, 893-899.) to monitor phenazine concentrations within bacterial cells in vivo both before and after reduction of the phenazine levels.

In some embodiments, one or more phenazine degrading agents herein described and in particular one or more engineered pyocyanin demethylase herein described can be used in methods and systems to interfere with viability of bacteria.

The term “bacterium” or “bacteria” as used herein refers to a prokaryotic microbial species of Gram-negative or Gram positive bacteria. The wording “Gram-negative bacteria” refers to bacteria that do not retain crystal violet dye in the Gram staining protocol. In contrast, the wording “Gram-positive bacteria” refers to those that are stained dark blue or violet by Gram staining. Exemplary bacteria in the sense of the disclosure comprise Pseudomonas, Brevibacterium, Coryneform Bacteria, Nocardia Brevibacterium linens, Brevibacterium, Burkholderia cenocepecia, Methanosarcina mazei, Mycobacterium abscessus, Pantoea agglomerans, Pectobacterium atrosepticum, Pelagio variabilis, Pseudomonas fluorescens, Streptomyces anulatus, Streptomyces cinnamonensis, and related species that produce phenazines to facilitate various physiological functions identifiable to a skilled person upon reading of the present disclosure.

In particular, in several embodiments, herein described, bacteria in the sense of the disclosure comprise phenazine producing bacteria, which comprise Pseudomonas aeruginosa and additional bacteria known or identifiable by a skilled person, and phenazine degrading bacteria which comprise Sphingomonas sp. DP58 (see Yang et al. Current Microbiology 2007, 55, 284-287 and Chen et al. Biodegradation 2008, 19, 659-667) and additional bacteria known or identifiable by a skilled person.

Identification of a phenazine degrading bacterium can be performed by various techniques. For example, identification of a phenazine producing bacterium can be performed by constructing a bacterial “enrichment culture” by defining a minimal growth medium where a phenazine (PCA, PYO, and additional phenazines identifiable by a skilled person) is provided as either (or both) the sole source of carbon or nitrogen. If growth is observed after many rounds of serial dilutions, phenazine-degraders can be isolated by plating the enrichment culture on an agar plate with the same medium composition or by dilution to extinction in liquid medium. Single colonies are picked, and streaked to fresh plates, and visually checked for purity. Once pure, the 16S rDNA is sequenced and the organism can be phenotypically characterized. Other methods for identifying a bacterium capable of phenazine degradation would be identifiable to a skilled person upon reading of the present disclosure.

In some embodiments, bacteria comprise persister cells which typically constitute a small portion of a culture which is tolerant to killing by lethal doses of bactericidal antibiotics. Persister bacterial cells can be identified, for example, by exposure of logarithmic or stationary cultures of the bacteria to antibiotics using concentrations exceeding five times the minimum inhibitory concentration for each antibiotic. Persister numbers can be determined by plating the antibiotic-treated cultures on LB agar plates and subsequent counting of colony forming units representing the cell numbers which survived antibiotic exposure. Other methods for identification of persister cells will be known by a skilled person, and can be found, for example, in Möker et al. (“Pseudomonas aeruginosa increases formation of multidrug-tolerant persister cells in response to quorum-sensing signaling molecules.” In J Bacteriol. 2010 April; 192 (7): 1946-55. Epub 2010 Jan. 22).

In some embodiments, one or more phenazine degrading agents herein described and in particular one or more engineered pyocyanin demethylases herein described can be used in methods and systems for the inactivation of phenazines and/or a phenazine related pathway.

The term “inactivation” as used herein with reference to a pathway refers to a complete or partial inhibition of one or more of the reactions or steps in the pathway.

The terms “inhibit” and “inhibition” as used herein refers to a decrease relative to a baseline level. Accordingly, inhibition of a reaction indicates a decrease in the relative output compared to an output selected as a baseline level. Inhibition of a reaction can be detected by detecting any products or other indicator and/or parameter associated with completion of the reaction and identifiable by a skilled person. Accordingly, an inactivated pathway in the sense of the present disclosure indicates a pathway in which any enzyme controlling a reaction in the pathway is biologically inactive or in which at least one of the reactions or steps of the pathway is otherwise inhibited, e.g. by degrading one or more enzymes of the pathway and/or by subtracting the relevant substrate and/or intermediate through phenazine degradation.

The term “pathway” as used herein refers to a biological process comprising one or more chemical or biological reactions or steps in which at least one substance is transformed, produced. and/or acquired by a bacterium. The one or more reactions or steps comprised in the pathway can involve molecules such as, for example, proteins, enzymes, cofactors, oxidizing/reducing agents, signaling molecules, metal ions, and others identifiable to a skilled person upon reading of the present disclosure that participate in the transformation, production and/or acquisition of the substance by a bacterium. In embodiments wherein pathway involves a bacterial cell signaling molecule, the pathway indicates signal transduction through cascade reactions of a series of signaling molecules as part of a complex system of communication that governs basic cellular activities and coordinates cell actions. Exemplary pathways of the disclosure comprise Fe (III) reduction to Fe (II) comprising the steps of reduction of Fe (III) to Fe (II) through the use of a reducing agent (such as, for example, pyocyanin, and/or other reducing agents), bacterial acquisition of Fe (II) comprising the steps of reduction of Fe (III) to Fe (II) by a reducing agent (such as, for example, pyocyanin, and/or other reducing agents) and importation of Fe (II) into the bacteria by a transporter protein (such as, for example, FeoB), and other pathways identifiable to a skilled person upon reading of the present disclosure.

The term “phenazine-related pathway” as used herein refers to either a pathway in which a phenazine is a starting material, intermediate, or product, or alternatively, any pathway in which at least one of the one or more of the steps comprised in the pathway are mediated by a phenazine. Exemplary pathways in which a phenazine is a starting material, intermediate, or product include, but are not limited to, phenazine biosynthesis, phenazine cycling, quorum sensing, and other pathways identifiable to a skilled person upon reading of the present disclosure. Exemplary pathways in which one or more of the steps of the pathway are promoted or mediated by a phenazine include, but are not limited to, reduction of Fe (III) to Fe (II) by phenazine, bacterial Fe (II) acquisition in which the Fe (II) is obtained, and other processes identifiable to a skilled person upon reading of the present disclosure.

In some embodiments, phenazine related pathways comprise phenazine-mediated bacterial biofilm formation, phenazine-mediated iron acquisition and phenazine mediated intracellular redox balancing of bacteria

In some embodiments, a phenazine related pathway comprises a phenazine-mediated signaling pathway of the bacteria. Specifically, in some embodiments, the bacteria have a motile and a sessile state and the signaling pathway triggers a transition from the motile to the sessile state.

In some embodiments, one or more phenazine related pathways comprise central metabolic pathways of the bacteria.

In some embodiments, the one or more phenazine related pathways comprise transportation of phenazines in and/or out of the bacterial cell. In other embodiments, phenazine related pathways comprise intracellular phenazine mediated redox hemostasis of the bacteria.

In some embodiments, a method and system to interfere with viability of bacteria is described, the method comprising contacting bacteria with one or more phenazine degrading agents herein described to reduce survivability and/or antibiotic resistance of the bacteria.

The term “viability” as used here in refers to whether or not a bacterial cell is able to maintain itself or recover its potentiality. Viable cells in the sense of the present disclosure are cells able to, or capable of recover the ability to form colonies and biofilms on or in a solid or liquid medium. In some embodiments, the term “medium” as used herein indicates an environment that is suitable to support growth of microorganisms or cells. In particular, suitable medium comprise growth medium or culture medium in a liquid or gel designed to support the bacteria in vitro, as well as tissues and other suitable environments within a host (including a human host) in vivo. Accordingly, various mediums are formed by or comprise medium components that are chemical compounds and molecules that are used in life-supporting functions and processes of bacteria, which allow bacterial cells to grow and reproduce.

Exemplary medium components comprise at least one redox-active compound in a solvent. In some embodiments, the solvent can comprise water in at least 10% by volume, preferably at least 50% by volume, and most preferably at least 95% by volume.

In some embodiments, the medium solvent can further comprise at least one organic solvent. Exemplary organic solvent includes ethanol, methanol, tetrahydrofuran, dimethylformamide (DMF), dimethyl sulfoxide (DMSO), acetic acid, formic acid, glycerol, glycol, isopropanol and 1-butanol.

In some embodiments, the redox-active compound has at least one oxidation absorption maximum in the wavelength of 400 nm to 700 nm in the oxidized state with a corresponding oxidation extinction coefficient. In some embodiments, the redox-active compound has at least one reduction absorption maximum in the wavelength of 400 nm to 700 nm in the reduced state with a corresponding reduction extinction coefficient.

In some embodiments, the at least one oxidation absorption maximum and the at least one reduction absorption maximum have an absorption shift or difference of at least 5 nm, preferably 50 nm and most preferably 150 nm.

In some embodiments, the ratio of oxidation extinction coefficient to the reduction extinction coefficient is at least 2 to 1, preferably at least 20:1 and most preferably 100:1.

In some embodiments, the ratio of reduction extinction coefficient to the oxidation extinction coefficient is at least 2 to 1, preferably at least 20:1 and most preferably 100:1.

In some embodiments, the at least one redox-active compound has a standard electrode potential E vs. NHE of −500 mV to 500 mV. As used herein, the term redox-active compound refers to a chemical compound that is able to undergo reversible electrochemical conversion between an oxidation state and reduction state. A redox-active compound includes naturally occurring redox-active molecules, organic, inorganic or metal ion complexes. Redox-active natural products include, but are not limited to those produced by the genera Streptomyces and Pseudomonas, including those redox-active natural products produced by P. aeruginosa, P. oryzihabitans, and P. luteola.

Exemplary redox-active compounds listed in Table 2.

TABLE 2
Exemplary redox-active compounds

| | | E^{0,} (vs. | # of Redox | | Reduction |
|---|---|---|---|---|---|
| Chemical name | Structure | NHE) | cycles over | Support | by |
| (Abbreviation) | (The oxidized form) | (mV) | 7 days | survival? | PA14? |
| Pyocyanin (PYO) | 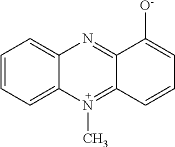 | −40 ^{a} | 31 | Yes | Yes |
| Phenazine-1- carboxylate (PCA) | 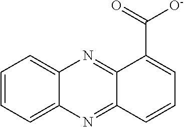 [SMILES: O=C([O-])c1cccc2nc3ccccc3nc12] | −114 ^{a} | 22 | Yes | Yes |
| 1-Hydroxyphenazine (1-OHPHZ) | 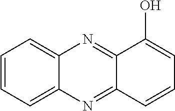 [SMILES: Oc1cccc2nc3ccccc3nc12] | −174 ^{a} | 14 | Yes | Yes |
| Methylene blue (MB) | 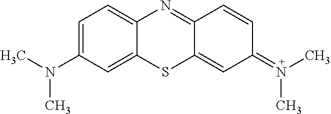 | 0 ^{b} (+11 ^{c}) | 3 | No | Yes |
| 2,6-AQDS | 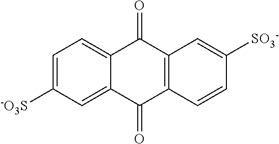 | −184 ^{d} | No cycle | No | Yes (very slowly) |
| Paraquat | 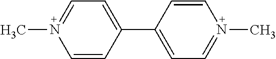 | −446 ^{e} | No cycle | No | No |
| Homogentisic acid (HMA) | 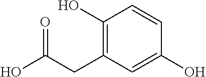 [SMILES: O=C(O)Cc1cc(O)ccc1O] | +306 ^{b} | No cycle | No | — |

^{a} Reference [20] [21]
^{b} E^{0,} values were measured in aqueous solution at pH 7 in this study
^{c} Reference (Fultz, M. L., and R. A. Durst. 1982. Mediator Compounds for the Electrochemical Study of Biological Redox Systems - a Compilation. Analytica Chimica Acta 140:1-18) [22]
^{d} Reference [23] [24]
^{e} References [25, 26] [27]

Additional medium components that can be found in a medium comprise amino acids. salts, polyacrylic acids, polyols, polyglycols, such as Polyethylene Glycols (e.g. PEG 1000, PEG 3000), polysaccharides, polypeptides, polynucleotides as well as other organic polymers with molecular weight between 10,000 to 1,000,000 Da and additional components identifiable by a skilled person. For example medium components can comprise sodium thioglycolate (HS—CH₂CO₂Na), sodium dithionite, Organic; simple sugars e.g. glucose, acetate or pyruvate; extracts such as peptone, tryptone, yeast extract etc., hydrogen carbonate salts (HCO₃⁻), amino acids, NH₄Cl, (NH₄)₂SO₄, KNO₃, KCl, K₂HPO₄, MgCl₂, MgSO₄, CaCl₂), Ca(HCO₃)₂, FeCl₃, Fe(NH₄)(SO₄)₂, Fe-chelates, CoCl₂, ZnCl₂, Na₂MoO₄, CuCl₂, MnSO₄, NiCl₂, NazSeO₄, Na₂WO₄, Na₂VO₄, Vitamins, amino acids, purines, pyrimidines

Methods for evaluating the viability of bacteria after the use of the methods and systems for interference with viability of bacteria described herein include, but are not limited to, measurement of colony forming units, cell counts such as that described by Wang et al. (J. Bacteriol. 2010, 192, 365-369) [28], and other methods identifiable to a skilled person upon the reading of the present disclosure.

In some embodiments, phenazine degrading agents, herein described, can be administered to enhance phenazine degradation endogenously and/or exogenously.

In particular, in some embodiments, enhancing phenazine degradation can be performed by expressing and/or delivering to the bacteria one or more phenazine degrading agents herein described. In an exemplary embodiment, a DNA sequence of a phenazine-degrading agent can be delivered by introduction of the DNA sequence into a bacterium via a vector (e.g. viral or plasmid vector), or other techniques identifiable by a skilled person upon reading of the present disclosure, and the DNA sequence expressed in the bacteria to produce the phenazine-degrading protein. In another embodiment, phenazine-degrading proteins can be expressed in other bacteria and then isolated and purified to afford phenazine-degrading proteins suitable for extracellular degradation of phenazine.

In embodiments wherein the phenazine degrading agent can be administered exogenously, any one of the engineered pyocyanin demethylase or derivative thereof herein described can be administered at suitable concentration to achieve inhibition of a phenazine degrading pathway, and/or to interfere with viability of bacteria and in particular inhibit viability of phenazine producing bacteria identifiable by detecting functionality of the pathway and/or viability of bacteria following contacting the bacteria with one or more of engineered pyocyanin demethylase or derivative thereof herein described. Additional methods to identify suitable concentrations of engineered pyocyanin demethylase or derivative thereof herein described are identifiable by a skilled person upon reading of the disclosure.

In some embodiments inhibition of a phenazine degrading pathway, and/or of viability of phenazine producing bacteria, can be performed in vitro or in vivo. Inhibition in vitro can be detected, by detecting decreased bacterial survival after treatments For in vivo applications, efficacy can be determined by healing rate of a chronic infection or through counts of bacteria in vivo.

In some embodiments concentration of engineered pyocyanin demethylase or derivative thereof herein described, suitable to achieve inhibition of a phenazine degrading pathway, and/or to interfere with viability of bacteria and in particular inhibit viability of phenazine producing bacteria identifiable comprise at least 0.001 uM, at least 0.01 uM, at least 0.1 uM, at least 1.0 uM, and more preferably at least 5.0 uM, at least 10.0 uM, and at least 20 uM.

In some embodiments, concentrations of a pyocyanin demethylase herein described capable of inhibition a phenazine degrading pathway, and/or of viability of phenazine producing bacteria can be in a range from 0.1 uM to 20 uM of PodA and can be combined with clinical concentrations of antimicrobials, which differ depending on the antimicrobial agent. Antimicrobial agents can be defined as any known compound that is being used with the intent to treat infections.

In some embodiments, a pyocyanin demethylase or derivative thereof herein described and in particular of an engineered pyocyanin demethylase or derivative thereof can be used in ranges of at least 0.75 uM at least 1 uM, at least 1.25 uM, at least 1.5 uM, at least 1.75 uM, at least 2 uM, at least 2.25 uM at least 2.5 uM at least 2.75 uM, at least 3 uM, at least 3.25 uM, at least 3.5 uM, at least 3.75 uM at least 4 uM, at least 4.25 uM, at least 4.5 uM, at least 4.75 uM and in additional concentrations identifiable by a skilled person upon reading of the present disclosure.

In embodiments where the inhibition is performed in vivo, the determination of a proper concentration can be performed in combination with clinical concentrations of antimicrobials, which differ depending on the antimicrobial agent.

In some embodiments, inactivating a phenazine or phenazine-related pathway comprises contacting the bacteria with one or more phenazine degrading agents to impair phenazine-mediated bacterial biofilm development in the bacteria.

As used herein the term “biofilm” indicates an aggregate of microorganisms in which cells adhere to each other on a surface. These adherent cells are frequently embedded within a self-produced matrix of extracellular polymeric substance (EPS). Biofilms can form on living or non-living surfaces and can be prevalent in natural, industrial and hospital settings. The microbial cells growing in a biofilm are physiologically distinct from planktonic cells of the same organism, which, by contrast, are single-cells that can float or swim in a liquid medium. Formation of a biofilm begins with the attachment of free-floating microorganisms to a surface. These first colonists adhere to the surface initially through weak, reversible adhesion via van der Waals forces. If the colonists are not immediately separated from the surface, they can anchor themselves more permanently using cell adhesion structures such as pili. When the biofilm growth is balanced with that of biofilm dispersion, the biofilm is considered “mature.” Methods to quantify and measure biofilms will be known to a skilled person and can include, for example, the COMSTAT method of Heydorn et al. (Microbiology 2000, 146, 2395-2407) [29].

In some embodiments, the phenazine-mediated bacterial biofilm development comprises phenazine-mediated iron acquisition of bacteria. Iron has been shown to be involved as a signal in bacterial biofilm formation (see, for example, Banin et al. PNAS, 2005, 102, 11076-11081) [30]. Phenazines have been shown to mediate iron acquisition in bacterial biofilm development, for example, by reduction of insoluble Fe (III) to more soluble Fe (II) (See, for example, Wang et al. J. Bacteriol. 2011, 193, 3606-3617) [31].

In some embodiments, one or more phenazine degrading agents herein described and in particular engineered pyocyanin demethylase and/or derivative thereof can be used to inhibit pathogenic microbial biofilm formation as well as to disrupt mature biofilm in vitro and in vivo (see Example 7).

In particular, in some embodiments, herein described phenazine degrading agents herein described can impact early stages in biofilm formation and development by decreasing the biofilm surface coverage.

In some embodiments, phenazine degrading agents herein described can disrupt the mature biofilm by interfering with anoxic growth of pathogens in deeper layers of the biofilm.

A person skilled in the art would understand that as biofilm matures, cells in deeper layers of the biofilm begin to experience oxygen limitation and redox stress, rendering the cells to be slow growing and highly resistant to antibiotics.

Thus, in some embodiments, a method for inhibiting bacteria biofilm formation and/or disrupting mature biofilm in a medium is described, the method comprising administering one or more phenazine degrading agents to the medium comprising the biofilm. The suitable medium comprises growth medium or culture medium in a liquid or gel designed to support the bacteria in vitro, as well as tissues and other suitable environments within a host (including a human host) in vivo.

The phenazine degrading agents such as PodA and a derivative thereof can degrade pyocyanin-like phenazines into 1-Hydroxyphenazine (1-OH—PHZ) which can limit the Fe concentration in pathogens such as P. aeruginosa by acting as an iron chelator, thus interfering with biofilm formation and/or maintenance

In some embodiments, functional derivative can be determined by assessing the proteins specific activity and kinetic parameters in comparison with a reference pyocyanin demethylase according to the disclosure and the related demethylase and biofilm inhibiting activity can be derived on that basis. For example, if a derivative of a pyocyanin demethylase has a specific activity and kinetic parameters comparable with those of WT PodA or PodA10, the derivative is expected to inhibit biofilms as will be understood by a skilled person upon reading of the present disclosure.

Thus, in some embodiments, another method for inhibiting bacteria biofilm formation and/or disrupting mature biofilm in a medium is described. The method comprises administering 1-hydroxyphenazine (1-OH—PHZ) as metal-chelating agent to the medium comprising the biofilm, alone or in combination with an antibiotic and/or other antimicrobial for a time and under conditions to reduce survivability and/or antibiotic resistance of the bacteria.

The term “chelator” or “chelating agent” as used herein refers to a molecule or ligand capable of binding a metal ion (e.g. iron) by forming multiple bonds to the metal. In particular, chelation involves the formation or presence of two or more separate coordinate bonds between a polydentate (multiple bonded) ligand and a single central metal. These ligands can be organic compounds, and are called chelants, chelators, chelating agents, or sequestering agents. Chelators can be molecules made by the host (such as, hemoglobin, transferrin, lactoferrin, conalbumin and ferritin); or molecules made by other microoorganisms (such as siderophores including Enterobactin, Yersiniabactin, Pyoverdine, Pyochelin, and others identifiable to a skilled person.); or synthetic molecules (e.g. deferoxamine, deferiprone, deferasirox, 2,2 dipyridyl, 1,10 phenanthroline, FerroZine, EDTA, diethylenetriamine, ethylene diamine, N,N′,N″-tris(2-pyridylmethyl)-1,3,5-cis,cis-triaminocyclohexane (tachpyr), and others identifiable to a skilled person).

In general, a metal chelator is a molecule capable of binding a metal and forming a molecular complex according to the generic reaction:

$$mM + lL + {hH}^{+} = {M}_{m} {L}_{l} {H}_{h} \qquad ( 2 )$$

wherein M is the metal, L is the chelator, and H⁺ is positive hydrogen ions with m, 1 and h independently equal to or higher than 1, and wherein the equilibrium constant βmlh is determined to be

$$β ml h ( M , L ) = \frac{[ {M}_{m} {L}_{l} {H}_{h} ]}{{{{[ M ]}^{m} [ L ]}^{l} [ {H}^{+} ]}^{h}} . \qquad ( 3 )$$

Some molecules are capable of binding more than one metal with different binding affinities which are reflected in different equilibrium constant Bmlh. In those instances, a molecule is known to a skilled person as a chelator of a particular metal when the molecule is capable of specifically binding that metal. Specific binding of a metal by a chelator is determined by the molecule binding with a highest equilibrium constant βmlh for the metal separately calculated with respect to other metals according to equation (3).

For example, some molecules capable of binding more than one metal. To determine whether a molecule is a Cu or Zn chelator, the equilibrium constant βmlh can be calculated for the molecule with respect to Cu, with respect to Zn and with respect to other metals. The calculated βmlh values can be compared to determine whether the molecule is a Cu) or Zn chelator.

1-hydroxyphenazine (1-OH—PHZ) in the sense of the present disclosure is a metal chelating agent. FIG. 27A of related application to U.S. Non-Provisional application Ser. No. 15/466,839 entitled “Phenazine Degrading Agents And Related Compositions, Methods And Systems For Interfering With Viability Of Bacteria” filed on Mar. 22, 2017 and granted on Feb. 9, 2019 with US patent number U.S. Pat. No. 10,913,936, illustrates a coordination geometry for 1-OH—PHZ metal complexation. 1-OH—PHZ can act as a bivalent metal chelator by forming a stable five-membered, 2:1, chelate compound. For example, 1-OH—PHZ can form metal complexes with bivalent metals such as Cu, Ni, Co, Zn and Cd (Kidani Y. Studies on Metal Chelate Compounds of Phenazine Derivatives. VIII. Metal Complexes of 1-Hydroxyphenazine, Yakugaku Zasshi. 1973 Sep.;93 (9): 1089-93.) [33]. The dissociation constant of 1-hydroxyphenazine with metals can be calculated according to equation 3 or determined by spectrophotometry or potentiometry as will be understood by a person skilled in the art. For example, in one exemplary embodiment, 1-hydroxyphenazine (1-OH—PHZ) as a copper chelating agent has a log of the equilibrium constant βmlh of binding copper equal to 8.68. (Kidani Y., Studies on Metal Chelate Compounds of Phenazine Derivatives. I. Spectrophotometric Studies on Copper Chelate Compounds of 1-Hydroxyphenazine and its Di-N-oxide. Chemical and Pharmaceutical Bulletin, Vol. 6 (1958) No. 5, P 556-562) [34])

In some embodiments, antimicrobials used in combination with the pyocyanin demethylase of the disclosure and/or suitable antibiotics, can comprise one or more chelating agents have concentrations ranging from I uM to 1000 μM, for example in amounts of 100 μM. 200 μM and in additional concentrations identifiable by a skilled person.

In some embodiments, phenazine degrading agents can be used in methods for treating and/or preventing a bacterial infection by a phenazine producing bacteria in an individual.

The term “treatment” as used herein indicates any activity that is part of a medical care for, or deals with, a condition, medically or surgically.

The term “prevention” as used herein indicates any activity which reduces the burden of mortality or morbidity from a condition in an individual. This takes place at primary, secondary and tertiary prevention levels, wherein: a) primary prevention avoids the development of a disease; b) secondary prevention activities are aimed at early disease treatment, thereby increasing opportunities for interventions to prevent progression of the disease and emergence of symptoms; and c) tertiary prevention reduces the negative impact of an already established disease by restoring function and reducing disease-related complications.

The term “condition” as used herein indicates a physical status of the body of an individual (as a whole or as one or more of its parts), that does not conform to a standard physical status associated with a state of complete physical, mental and social well-being for the individual. Conditions herein described include but are not limited to disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms.

The term “individual” as used herein in the context of treatment includes a single biological organism, including but not limited to, animals and in particular higher animals and in particular vertebrates such as mammals and in particular human beings.

In some embodiments, the method for treating and/or preventing a bacterial infection in an individual comprises administering to the individual an effective amount of one or more phenazine degrading agents herein described alone or in combination with an antibiotic and/or other antimicrobial. In some embodiments, administering of one or more phenazine degrading agents can be performed in combination with one or more antibiotics and/or other antimicrobials. In particular, the phenazine degrading agents, herein described, will be selected by the skilled person as not interfering in a deleterious manner with the normal biochemical pathways of the individual.

Exemplary antibiotics that can be used in combination with the one or more phenazine degrading agents herein described include Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam. As a person of ordinary skill in the art would understand, the antibiotics herein listed can be selected for treating infections or reducing inflammation caused by bacteria including Staphylococcus aureus, Pseudomonas (P. aeruginosa), Burkholderia cepacia, some mycobacteria.

The administering to the individual the one or more phenazine degrading agents alone or in combination with an antibiotic and/or other antimicrobial can be performed through various administration routes including oral ingestion, inhalation, intranasal, topical application, intravenous or subcutaneous injections and others as will be recognized by a person skilled in the art. The one or more phenazine degrading agents alone or in combination with an antibiotic and/or other antimicrobial can be in a form of an aqueous solution, cream, solid powder, tablets, aerosols, or other forms as will be understood by a person skilled in the art.

In some embodiments, an antimicrobial is described. The antimicrobial comprises one or more phenazine degrading agents herein described. The one or more phenazine degrading agents are in particular comprised in the antimicrobial in an amount suitable to reduce antibiotic resistance and/or survivability of phenazine producing bacteria. In some embodiments, the antimicrobial comprises a compatible vehicle, which can be a vehicle for effective administrating and/or delivering of the one or more agents to an individual. In some embodiments of the methods and systems, the bacteria comprise persister cells.

An “antimicrobial” as described herein indicates a substance that kills or inhibits the growth of microorganisms such as bacteria, fungi, or protozoans. Antimicrobial either kills microbes (microbiocidal) or prevent the growth of microbes (microbiostatic).

In some embodiments, the antimicrobial comprises one or more phenazine degrading agents optionally a compatible vehicle for effective administrating and/or delivering of the one or more agents to an individual.

The term “vehicle” as used herein indicates any of various media acting usually as solvents, carriers, binders or diluents for phenazine degrading agents comprised in the composition as an active ingredient.

In some embodiments, antimicrobial is a pharmaceutical composition comprising one or more phenazine degrading agents for the treatment of cystic fibrosis and a pharmaceutically acceptable vehicle such as an excipient or diluent.

The term “excipient” as used herein indicates an inactive substance used as a carrier for the active ingredients of a medication. Suitable excipients for the pharmaceutical compositions herein disclosed include any substance that enhances the ability of the body of an individual to absorb the one or more agents. Suitable excipients also include any substance that can be used to bulk up formulations with the one or more agents to allow for convenient and accurate dosage. In addition to their use in the single-dosage quantity, excipients can be used in the manufacturing process to aid in the handling of the one or more agents. Depending on the route of administration, and form of medication, different excipients may be used. Exemplary excipients include but are not limited to anti-adherents, binders, coatings disintegrants, fillers, flavors (such as sweeteners) and colors, glidants, lubricants, preservatives, or sorbents.

The term “diluent” as used herein indicates a diluting agent which is issued to dilute or carry an active ingredient of a composition. Suitable diluents include any substance that can decrease the viscosity of a medicinal preparation.

In particular, in some embodiments, the phenazine degrading agents herein described can be included in pharmaceutical compositions which contain at least one phenazine degrading agents herein described, in combination with one or more compatible and pharmaceutically acceptable vehicles, and in particular with pharmaceutically acceptable diluents or excipients. In those pharmaceutical compositions, the phenazine degrading agents can be administered as an active ingredient for treatment or prevention of a condition in an individual.

In some embodiments, the antimicrobial or pharmaceutical composition comprising one or more phenazine degrading agents herein described further comprises antibiotic and/or an additional antimicrobial.

The term “antibiotics” as used herein refers to a type of antimicrobial used in the treatment and prevention of bacterial infection. Some antibiotics can either kill or inhibit the growth of bacteria. Others can be effective against fungi and protozoans. The term “antibiotics” can be used to refer to any substance used against microbes. Antibiotics are commonly classified based on their mechanism of action, chemical structure, or spectrum of activity. Most antibiotics target bacterial functions or growth processes. Antibiotics having bactericidal activities target the bacterial cell wall, such as penicillins and cephalosporins, or target the cell membrane, such as polymyxins, or interfere with essential bacterial enzymes, such as rifamycins, lipiarmycins, quinolones and sulfonamides. Antibiotics having bacteriostatic properties target protein synthesis, such as macrolides, lincosamides and tetracyclines. Antibiotics can be further categorized based on their target specificity. “Narrow-spectrum” antibacterial antibiotics target specific types of bacteria, such as Gram-negative or Gram-positive bacteria. “Broad-spectrum” antibiotics affect a wide range of bacteria.

In some embodiments, suitable antibiotics that can be used in the antimicrobial in combination with Fe chelators include ampicillin, kanamycin, ofloxacin, Aminoglycosides, Carbapenems, Ceftazidime, Cefepime, Ceftobiprole, Fluoroquinolones, Piperacillin, Ticarcillin, tobramycin, aztreonam, coliston, tazobactam, and others (or combinations of these antibiotics) that can be recognized by a person skilled in the art.

In some embodiments, suitable antibiotics comprise antibiotics effective against pathogen Pseudomonas aeruginosa such as Aminoglycosides, Carbapenems, Ceftazidime, Cefepime, Ceftobiprole, Fluoroquinolones, Piperacillin, Ticarcillin, tobramycin, aztreonam, coliston, and others (alone or in combination) that can be recognized by a skilled person.

Additional antibiotics suitable in particular for treatment of cystic fibrosis include Amoxicillin and clavulanic acid (Augmentin®), Methicillin, oxacillin, nafcillin, cloxacillin, dicloxacillin, cabenicillin, ticarcillin, piperacillin, mezlocillin, azlocillin, ticarcillin and clavulanic acid (Timentin®), piperacillin and tazobactam (Zosyn®), cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam. A person skilled in the art would be able to select appropriate antibiotics for treating cystic fibrosis caused by particular pathogen. An exemplary indication of antibiotic, is shown in Table 3 below From Orenstein, D. Cystic Fibrosis: A Guide for Patient and Family, 4th ed. LWW; 2011.

TABLE 3
An exemplary list of antibiotics

| | | |
|---|---|---|
| Type and kinds | Bacteria Treated | How Taken |

Penicillins

| | | |
|---|---|---|
| Amoxicillin and clavulanic | Staphylococcus | |
| acid (Augmentin ®) | aureus (Staph) | |
| Methicillin, oxacillin | Pseudomonas | Intravenous, |
| and nafcillin | (P. aeruginosa) | intramuscular |
| Cloxacillin and | Staph | Oral |
| dicloxacillin | | |
| Cabenicillin, | P. aeruginosa | Intravenous |
| ticarcillin, piperacillin, | | |
| mezlocillin and azlocillin | | |
| Ticarcillin and clavulanic | Staph, P. aeruginosa | intravenous |
| acid (Timentin ®) | | |
| Piperacillin and | P. aeruginosa | intravenous |
| tazobactam (Zosyn ®) | | |

Cephalosporins

| | | |
|---|---|---|
| Cephalexin, cefdinir, | Staph, P. aeruginosa | oral |
| cefprozil and cefaclor | | |
| Cefuroxime | Staph | oral |

Sulfa

| | | |
|---|---|---|
| Sulfisoxazole | P. aeruginosa | oral |
| Erythromycin/sulfisoxazole | Staph | oral |

Aminoglycosides

| | | |
|---|---|---|
| Tobramycin, amikacin, | P. aeruginosa | Intravenous, |
| gentamicin | (in combination | inhaled |
| | with gentamicin, | |
| | tobramycin, and | |
| | amikacin; also | |
| | work well with | |
| | anti-Pseudomonas | |
| | penicillin | |
| | drug) | |

Macrolides

| | | |
|---|---|---|
| Erythromycin, | Staph and may | Oral, |
| clarithromycin | help reduce | intravenous |
| and azithromycin | inflammation from | |
| | P. aeruginosa | |

Tetracyclines

| | | |
|---|---|---|
| Tetracycline, | Formerly | Oral, |
| doxycycline, | P. aeruginosa, some | intravenous, |
| minocycline, | Burkholderia | intramuscular |
| and tigecycline | cepacian and Staph | |

Quinolones

| | | |
|---|---|---|
| Ciprofloxacin, levofloxacin | Pseudomonas | Oral, intravenous |

Vancomycin

| | | |
|---|---|---|
| Vancomycin | Staph and | intravenous |
| | methicillin-resistant | |
| | Staphylococcus | |
| | aureus (MRSA) | |

Linezolid

| | | |
|---|---|---|
| Linezolid | MRSA and some | Oral, |
| | mycobacteria | intravenous |

Imipenem & Meripenem

| | | |
|---|---|---|
| Imipenem & Meripenem | P. aeruginosa, Staph | intravenous |

Aztreonam (Cayston ®)

| | | |
|---|---|---|
| Aztreonam (Cayston ®) | P. aeruginosa | Intravenous, |
| | | inhaled |

In embodiments herein described concentration of suitable antibiotics that can be used in the antimicrobial against phenazine producing bacteria can identified based on the respective breakpoint Minimum Inhibitory Concentration (MIC)

The wording breakpoint minimum inhibitory concentration (MIC) indicates the concentration that inhibits visible bacterial growth at 24 hours of growth in specific media, at a specific temperature, and at a specific carbon dioxide concentration. Methods that can be used to measure the MIC of a microorganism comprise broth dilution, agar dilution and gradient diffusion (the ‘E test’), where twofold serial dilutions of antibiotic are incorporated into tubes of broth, agar plates or on a paper strip, respectively, as will be understood by a person skilled in the art. The disk diffusion method defines an organism as susceptible or resistant based on the extent of its growth around an antibiotic-containing disk. MIC values are influenced by several laboratory factors.

Laboratories follow standard for parameters such as incubation temperature, incubation environment, growth media, as well as inoculum and quality control parameters. In the U.S. Standards for determining breakpoint MIC values for various bacteria can be found in Clinical & Laboratory Standards Institute (CLSI) publications, with an example also provided as Appendix A of U.S. Provisional Application No. 62/722,124 incorporated herein by reference in its entirety, as will be understood by the skilled person. In Europe, standards for determining breakpoint MIC values for bacteria can be found in European Committee on Antimicrobial Susceptibility Testing (EUCAST) see website: www.eucast.org/clinical_breakpoints/at the time of filing of the instant disclosure) as will be understood by the skilled person.

In some embodiments, in methods and systems herein described and related compositions one or more antibiotics can be administered in concentration of at least 0.00005 ug mL, preferably at least 0.002 ug mL, at least 0.01 ug mL, at least 0.025 ug mL, or at least 0.08 ug mL. or at least 0.1 ug mL, and in additional concentrations identifiable by a skilled person upon reading of the present disclosure. The specific concentration of each antibiotic can be determined based on the related MIC as will be understood by a skilled person.

In most preferred embodiments of methods and systems of the present disclosure, one or more antibiotics can be administered at a concentration of at least 2.0 ug mL, at least 10.0 ug mL, at least 25.0 ug mL, at least 50.0 ug mL, and at least 100.0 ug mL-1, in particular in combination with concentration of one or more engineered pyocyanin demethylase in concentration associated with a resulting synergic inhibition of bacteria viability herein described. The specific concentration of each antibiotic can be determined based on the related MIC as will be understood by a skilled person.

In most preferred embodiments, antibiotics used in methods and systems and related compositions of the present disclosure are aminoglycosides. The term “aminoglycosides” as used herein indicates an antibiotic that inhibit protein synthesis and contain an amino-modified glycoside aa portion of the molecule. Aminoglycoside antibiotics are typically used as a Gram-negative antibacterial medication, more typically against Gram-negative aerobes. such as Pseudomonas, Acinetobacter, and Enterobacter as well as some Mycobacteria, including the bacteria that cause tuberculosis, as well be understood by a skilled person.

Aminoglycosides antibiotics can be categorized based on the molecular structure in 4,6-disubstituted deoxystreptamine sub-class of aminoglycosides, the neomycins are examples of the 4,5-disubstituted sub-class, and a non-deoxystreptamine aminoglycoside subclass. Aminoglycosides antibiotics are typically administered intravenously and intramuscularly, topical preparations for wounds, oral administration for gut decontamination (e.g., in hepatic encephalopathy) and/or a nebulized form

Exemplary aminoglycoside antibiotics comprise, Kanamycin A Amikacin, Tobramycin, Dibekacin, Gentamicin, Sisomicin, Netilmicin, Neomycins B, C, Streptomycin and Plazomicin wherein kanamycin A through netilmicin are examples of the 4,6-disubstituted deoxystreptamine sub-class of aminoglycosides, the neomycins are examples of the 4,5-disubstituted sub-class, and streptomycin is an example of a non-deoxystreptamine aminoglycoside. Tobramycin is an exemplary representative of aminoglycosides as will be understood by a skilled person.

In some preferred embodiments methods and systems as well as related compositions according to the disclosure are performed to provide a combined administration of a phenazine degrading agent and an antibiotic at combined concentrations resulting in a synergistic inhibition of one or more target phenazine producing bacteria. In those embodiments, a naturally occurring and isolated pyocyanin demethylase PodA and/or an engineered pyocyanin demethylase PodA can be administered at a concentration of at least 0.01 uM, 0.1 uM, 1.0 uM, 5.0 uM, 10.0 uM, and 20 uM and the antibiotic can be administered at a concentration of at least 2.0, at least 10.0 ug mL-1, at least 25.0 ug mL-1, at least 50.0 ug mL-1, and at least 100.0 ug mL.

As a person skilled in the art would appreciate, pyocyanin-like phenazine as a natural pigment can change its color from blue to yellow upon reduction, thus can be used as bacterial pigments for applications in food, pharmaceutical, cosmetics, paint, and textile industries. Thus, in some embodiments, the phenazine degrading agents herein described can be administered to a medium comprising pyocyanin-like phenazine pigments in absence of bacteria as a color-control agent to control the color of the medium.

In some embodiments, a composition can comprise one or more phenazine degrading agents herein described with one or more medium components.

In some embodiments, the composition can comprise medium components such as sodium thioglycolate (HS—CH₂CO₂Na), sodium dithionite, organic molecules such as simple sugars e.g. glucose, acetate or pyruvate; extracts such as peptone, tryptone, yeast extract etc., hydrogen carbonate salts (HCO₃), amino acids, NH₄Cl, (NH₄)₂SO₄, KNO₃, KCl, K₂HPO₄, MgCl₂, MgSO₄, CaCl₂, Ca(HCO₃)₂, FeCl₃, Fe(NH₄)(SO₄)₂, Fe-chelates, CoCl₂, ZnCl₂, Na₂MoO₄, CuCl₂, MnSO₄, NiCl₂, Na₂SeO₄, Na₂ WO₄, Na₂ VO₄, Vitamins, amino acids, purines, pyrimidines.

In some embodiments, the composition can comprise a basic binder, and an isocyanate compound and in particular an isocyanate pre-polymer. In some embodiments, the composition can comprise an aqueous dispersion of an acryl-modified polyester resin, a blocked polyisocyanate compound having a nonionic hydrophilic group; and an aqueous dispersion of acrylic-based polymer fine particles.

In several embodiments, the composition comprising one or more phenazine degrading agent and one or more medium component can act as a barrier against environmental conditions. Such chemical composition can contain one or more pigments to impact color and opacity, binder polymer forming a matrix to hold the one or more pigments in place, extender to improve adhesion, solvent such as organic solvent or water to reduce the viscosity of the paint, additives to modify the properties of the paint and other ingredients identifiable to a person skilled in the paint industry.

As described herein, the phenazine degrading agents, bacteria, antimicrobial agents or compositions herein described can be provided as a part of systems to perform any methods, including any of the assays described herein. In some embodiments, a system can comprise one or more phenazine degrading agents alone or in combination with pyocyanin-like phenazine in a certain proportion to produce a desired color.

In embodiments of systems where detection can be performed, the systems can be provided in the form of arrays or kits of parts. An array, sometimes referred to as a “microarray”. can include any one, two or three dimensional arrangement of addressable regions bearing a particular molecule associated to that region. Usually, the characteristic feature size is micrometers.

In a kit of parts, the phenazine degrading agents, antimicrobial agent, candidate phenazine degrading agents, bacteria and compositions and other reagents to perform the method can be comprised in the kit independently. In particular, the phenazine degrading agents, antimicrobial agent, candidate phenazine degrading agents, bacteria can be included in one or more compositions, and each phenazine degrading agent can be in a composition together with a suitable vehicle. In some embodiments, a kit can comprise a phenazine degrading agent with medium components within a composition herein described.

Additional components can include labeled molecules and in particular, labeled polynucleotides, labeled antibodies, labels, microfluidic chip, reference standards, and additional components identifiable by a skilled person upon reading of the present disclosure. The terms “label” and “labeled molecule” as used herein as a component of a complex or molecule referring to a molecule capable of detection, including but not limited to radioactive isotopes, fluorophores, chemiluminescent dyes, chromophores, enzymes, enzyme substrates, enzyme cofactors, enzyme inhibitors, dyes, metal ions, nanoparticles, metal sols, ligands (such as biotin, avidin, streptavidin or haptens) and the like. The term “fluorophore” refers to a substance or a portion thereof which is capable of exhibiting fluorescence in a detectable image. As a consequence, the wording “labeling signal” as used herein indicates the signal emitted from the label that allows detection of the label, including but not limited to radioactivity, fluorescence, chemiluminescence, production of a compound in outcome of an enzymatic reaction and the like.

In some embodiments, detection of a viable bacteria can be carried either via fluorescent based readouts, in which the labeled antibody is labeled with fluorophore, which includes, but not exhaustively, small molecular dyes, protein chromophores, quantum dots, and gold nanoparticles. Additional techniques are identifiable by a skilled person upon reading of the present disclosure and will not be further discussed in detail.

In particular, the components of the kit can be provided, with suitable instructions and other necessary reagents, in order to perform the methods here described. The kit will normally contain the compositions in separate containers. Instructions, for example written or audio instructions, on paper or electronic support such as tapes or CD-ROMs, for carrying out the assay, will usually be included in the kit. The kit can also contain, depending on the particular method used, other packaged reagents and materials (i.e. wash buffers and the like).

The methods, herein described, can be performed in vivo and/or in vitro as will be understood by a skilled person.

### EXAMPLES

The stabilized phenazine degrading agents and related compositions, methods and systems herein disclosed are further illustrated in the following examples, which are provided by way of illustration and are not intended to be limiting.

In particular, the following examples illustrate exemplary stabilized PodAs, as well as related methods and protocols for degrading phenazine. A person skilled in the art will appreciate the applicability and the necessary modifications to adapt the features described in detail in the present section, to additional phenazine degrading agents and related compositions, methods and systems according to embodiments of the present disclosure. The following materials and methods were used.

Bacterial strains, culture media, and chemicals. Strains used in this study are listed in Table 4 below

TABLE 4
Bacterial strains used in this study

| | | |
|---|---|---|
| Strain | Relative Genotype | Source |

E. coli strains

| | | |
|---|---|---|
| E. coli DH5α | Φ80dlacZ ΔM15 recA1 | NEB |
| | endA1 gyrA96 thi-1 hsdR17 | |
| | (rₖ−, mₖ⁺) supE44 relA1 deoR | |
| | Δ (lacZYA-argF) U169 phoA | |
| E. coli BL21 (□DE3) | B F⁻ ompT gal den) lon hsdS | NEB |
| | (r_{B}m_{B}) gal (ΔDE3) | |

P. aeruginosa strains

| | | |
|---|---|---|
| DKN263 | Wildtype UCBPP-14 | Lab Collection |

Escherichia coli BL21 (ADE3) (41) [36] was used for protein overexpression and E. coli DH5a (New England Biolabs) was used for plasmid construction. All E. coli strains were grown at 37° C. in lysogeny broth (LB, Difco™) or Terrific Broth (TB, Difco™). Ampicillin for culturing E. coli was used at 100 ug mL-1. Pseudomonas aeruginosa strain UCBPP-PA14 (hereafter Pseudomonas aeruginosa aeruginosa) was used for growth analyses and cultivated on LB. All chemicals were purchased from Sigma-Aldrich unless otherwise noted; glycerol (VWR), HEPES (Gold BioTechnology), sodium chloride (Fisher Scientific).

Plasmid construction. All plasmids used in this work are listed in Table 5, below

TABLE 5
Plasmids used in this study

| Plasmid | Genotype | Source |
|---|---|---|
| pTEV16 | lad⁺ bla⁺ | (1) |

Overexpression plasmids

| | | |
|---|---|---|
| pET20b(+)- | pET20b(+)-podA D68A | pET20b(+)- |
| podA D68A | | podA D68A |
| pET20b(+)- | pET20b(+)-podA D72A | pET20b(+)- |
| podA D72A | | podA D72A |
| pET20b(+)- | pET20b(+)-podA H121A | pET20b(+)- |
| podA H121A | | podA H121A |
| pET20b(+)- | pET20b(+)-podA E154A | pET20b(+)- |
| podA E154A | | podA E154A |
| pET20b(+)- | M. fortuitum PodA^{Y156A} in pET20b | (2) |
| podA Y156A | (+) | |
| pPodA1 | Mycobacterium fortuitum podA₄₁₋₁₆₂ + | This study |
| | cloned into pTEV16 with a TEV | |
| | cleavable 6X-His tag | |
| pPodA2 | Mycobacterium fortuitum podA | This study |
| | designed variant cloned into | |
| | pTEV16 with a TEV cleavable 6X- | |
| | His tag: I73K, A87V, T91V, M99T, | |
| | A128V | |
| pPodA3 | Mycobacterium fortuitum podA | This study |
| | designed variant cloned into | |
| | pTEV16 with a TEV cleavable 6X- | |
| | His tag: I73T, A87V, T91V, M99V, | |
| | A128V | |
| pPodA4 | Mycobacterium fortuitum podA | This study |
| | designed variant cloned into | |
| | pTEV16 with a TEV cleavable 6X- | |
| | His tag: I73K, A87V, M99T, | |
| | A129V, K141T | |
| pPodA5 | Mycobacterium fortuitum podA | This study |
| | designed variant cloned into | |
| | pTEV16 with a TEV cleavable 6X- | |
| | His tag: I73E, A87V, M99I, A129V, | |
| | K141T | |
| pPodA6 | Mycobacterium fortuitum podA | This study |
| | designed variant cloned into | |
| | pTEV16 with a TEV cleavable 6X- | |
| | His tag: I73L, A87I, M99V, A129V, | |
| | K141T | |
| pPodA7 | Mycobacterium fortuitum podA | This study |
| | designed variant cloned into | |
| | pTEV16 with a TEV cleavable 6X- | |
| | His tag: I73R, A87V, T91V, M99V, | |
| | A129T | |
| pPodA8 | Mycobacterium fortuitum podA | This study |
| | Sarel designed variant cloned into | |
| | pTEV16 with a TEV cleavable 6X- | |
| | His tag: I73L, A87I, T91V, M99T, | |
| | A129V | |
| pPodA9 | Mycobacterium fortuitum podA | This study |
| | designed variant cloned into | |
| | pTEV16 with a TEV cleavable 6X- | |
| | His tag: A53N, I73R, A87V, T91V, | |
| | A129V | |
| pPodA10 | Mycobacterium fortuitum podA | This study |
| | designed variant cloned into | |
| | pTEV16 with a TEV cleavable 6X- | |
| | His tag: I73K, A87V, T91V, | |
| | A129V, K141T | |
| pPodA11 | Mycobacterium fortuitum podA | This study |
| | designed variant cloned into | |
| | pTEV16 with a TEV cleavable 6X- | |
| | His tag: A53N, I73T, A87V, M99V, | |
| | A129T | |

Primers were synthesized by Integrated DNA Technologies, Inc. (IDT [San Diego, CA]) and are listed in Table 6, below.

TABLE 6
Primers used in this study

| | Primer | SEQ |
|---|---|---|
| | Sequence | ID |
| Primer Name | 5′ → 3′ | NO |

Overexpression primers

| | | |
|---|---|---|
| 5′ pod A pTEV 16 | NNGCTCTTCNTTCAT | 5 |
| | GGACGGTCGCGGCGG | |
| | CCGGAGTACAA | |
| 3′ podA pTEV16 | NNGCTCTTCNTAATC | 6 |
| | ATTTCGTCAGTTTCA | |
| | ATTCGTACTTCTC | |
| 5′ pTEV16 gibson | GCCCTGAAAATACAG | 7 |
| | GTTTTCACTAGTTG | |
| 3′ pTEV16 gibson | TAAGAATTCTCGAG | 8 |
| | CTCCCGGGATC | |
| 5′ podA gBlock | ATTACGATATCCCAA | 9 |
| gibson | CTAGTGAAAACCTGT | |
| | ATTTTCAGGGC | |
| 3′ podA gBlock | TGCTCAGCGGCCGCG | 10 |
| gibson | GATCCCGGGAGCTCG | |
| | AGAATTCTTA | |

For heterologous protein expression, the gene coding for PodA was amplified from Mycobacterium fortuitum strain ATCC 6841 using Phusion™ High Fidelity DNA Polymerase (ThermoFisher Scientific) per manufactures instructions and cloned into an IPTG inducible overexpression vector, pTEV16 (42), using Type II-Restriction BspQI cloning (43) [38]. The resulting plasmid was referred to as pPodA1. Catalytic PodA variants were utilized from a previous study (12) [1]. For designed proteins gBlocks® were synthesized from IDT to code for relevant amino acid substitutions, with 5′ and 3′ basepairs corresponding to the MCS of pTEV16. One set of primers was used to amplify each gBlock® and another set to amplify pTEV16. PCR constructs were isolated using the Monarch® PCR & DNA Cleanup Kit (NEB). Genes were then cloned into pTEV16 using Gibson Assembly® Master Mix (NEB) per manufacturer protocol. The resulting plasmids pPodA2-pPodAllare listed in Table 5.

Protein overproduction and purification of soluble and inclusion body fractions. Plasmids coding for proteins of interest were transformed into E. coli BL21 (ADE3). Overnight cultures (10 mL) of transformants were sub-cultured (1% [v/v]) into 10 L of TB containing ampicillin. Cultures were grown at 37° C. with shaking to an optical density of 0.4 (OD (,mm) and plasmid expression was induced with IPTG (50 μM) and shaken overnight at 16° C. Cells were harvested by centrifugation at 5,000×g for 10 min in a Beckman Coulter Avanti J-20 XOI refrigerated centrifuge using a JLA-8.1000 rotor at 4° C. Cell pellets were stored at −80° C. until used.

For purification, cell pellets were thawed and re-suspended in 30 mL Buffer A (HEPES [50 mM, pH 7.5], NaCl [500 mM], and imidazole [20 mM]) containing lysozyme (1 mg/mL) DNase (5 ug mL-1), and protease inhibitor (PMSF, 0.5 mM). Cells were lysed by four passages through an Avestin Emulsiflex C3 (ATA Scientific) at 15,000 psi. Due to difficulties filtering lysate in subsequent steps, additional DNase (5 ug mL-1) was added before centrifugation. Clarified lysates were obtained via centrifugation at 40,000×g for 30 min at 4° C. in a Beckman Coulter Avanti centrifuge with the JA-25.50 rotor and filtered through a 0.45 μm filter (Millipore). Using an AKTA FPLC, lysate was applied to a pre-equilibrated 5 mL HisTrap™ FF (Sigma-Aldrich) after which the column was washed with 10 CV of Buffer A, 7 CV of 8% Buffer B (HEPES [50 mM. pH 7.5], NaCl [500 mM], and imidazole [500 mM]), and a gradient to 100% Buffer B over 10 CV. Fractions were run on an SDS-PAGE gel and fractions containing PodA were combined and cleaved for 3 h at 25° C. with TEV protease (1:50 mg:mg ratio of TEV to PodA). TEV was purified and stored as described previously (44) [39]. Cleaved protein was dialyzed at 4° C. for 3 h in HEPES (50 mM, pH 7.5), NaCl (500 mM), EDTA (1 mM), followed by dialysis against HEPES (50 mM. pH 7.5), NaCl (500 mM), and imidazole (20 mM). Cleaved PodA was separated from TEV and contaminates by passage over a HisTrap™ FF that was equilibrated with Buffer A. For the final dialysis, PodA was dialyzed against 50 mM HEPES (pH 7.5), 100 mM NaCl, and 20% glycerol (v/v).

For purification of PodA from inclusion bodies, the pellets remaining following lysis and centrifugation at 40,000×g were resuspended in 20 mL of detergent buffer [50 mM HEPES (pH 7.5) and 1% Triton-X (v/v)] using a Bio-gen PRO200 cell homogenizer (PRO Scientific). Sample was spun for 15 min at 40,000×g at 4° C. in a Beckman Coulter Avanti centrifuge with a JA-25.50 rotor. Supernatant was discarded and the remaining cell pellet was resuspended in 5 mL of buffer [50 mM HEPES (pH 7.5), 500 mM NaCl] using a cell homogenizer, supplemented with lysozyme (200 ug mL-1), and incubated for 10 min. Volume was increased to 20 mL with HEPES (50 mM, pH 7) and sample was spun for 40,000×g at 4° C. for 15 min. Supernatant was discarded and pellet was homogenized in 10 mL of denaturing buffer 50 mM HEPES (pH 7.5), 500 mM NaCl, and 8M urea) and incubating rocking overnight. The sample was spun at for 40,000×g at 25° C. for 30 min and supernatant was incubated with 1 mL HisPur Ni-NTA resin (Thermo Fisher Scientific) on a rocking platform for 10 min. Beads were allowed to settle without shaking, supernatant was discarded, and resin was pipetted into glass gravity columns. Resin was washed with 15 mL of denaturing buffer, followed by an elution with denaturing buffer supplemented with imidazole (500 mM). Fractions were analyzed by SDS-PAGE and those containing protein were combined into SnakeSkin 3.5 K MWCO dialysis tubing (Thermo Fisher Scientific). Urea was slowly dialyzed out in HEPES (50 mM, pH 7.5) and NaCl (500 mM) by lowing urea concentrations by 1 M increments. Each step was dialyzed at RT for 30 min. Protein was dialyzed in HEPES (50 mM, pH 7.5), NaCl (500 mM), and imidazole (20 mM) overnight at 4° C. The following day, protein was cleaved for 3 h at 25° C. with TEV protease (1:50 mg:mg ratio of TEV to PodA). The sample was applied to a HisPur gravity column and cleaved protein in the flow thru was confirmed by SDS-PAGE. Cleaved PodA protein was then dialyzed for storage for 3 h at 4° C. in HEPES (50 mM pH 7.5), NaCl (250 mM) and finally in HEPES (50 mM, pH 7.5), NaCl (100 mM), and glycerol (20%, v/v). Protein concentration was established on a NanoDrop™ using the extinction coefficient and MW of each protein. Protein was drop frozen in LN2 and stored at −80° C. until use.

Protein design calculations. Using the PodA structure as a starting point (PDB entry 5k21), 13 positions was selected in the homotrimeric interfaces for design: 53Ala, 64Val, 67Met, 73Ile, 87Ala, 91Thr, 92Asn, 99Met, 112Glu, 118Leu, 129Ala, 134Thr, 141Lys. As previously described (24, 25), a Position Specific Scoring Matrix (PSSM) was generated using the default parameters and calculated the tolerated sequence identities at each of the 13 positions with PSSM cutoff≥−2 and Rosetta ΔΔG<6 R.e.u. Next, it was enumerated, modeled and refined in Rosetta all the possible combinations of mutations allowed by the tolerated sequence space that differed from the WT protein by 3 to 5 mutations (a total of 118,424 combinations of mutations). During all Rosetta modeling and design calculations, the structure was modeled with the Ca²⁺ ions and their ligating residues unchanged and subject to C3 symmetry constraints. The designs were ranked according to Rosetta energy and clustered them, retaining the low-energy designs that exhibited at least two mutations relative to one another. For experimental screening 10 mutants out of the top 35 were chosen by visual inspection.

A web-accessible version of AffiLib (for non-symmetric design) is available at the web page (//AffiLib.weizmann.ac.il) for academic users.

Synthesis of Pyocyanin (PYO). PYO was synthesized from phenazine methosulfate using a protocol described previously (45) [42]. Deviations included eliminating purification of PYO via TLC plates and utilizing dichloromethane in the place of chloroform. PYO was analyzed for purity via HPLC analysis and was found to be >95% pure. Lyophilized PYO was stored at 4° C. until use, after which it was resuspended in 20 mM HCl to make a 5 mM PYO stock, filtered to rid of insoluble particulate, and PYO stock concentration was calculated by measuring the absorbance at 690 nm and using the extinction coefficient of PYO (4130 M⁻¹ cm⁻¹) in combination with Beer's Law (A=εcl).

Specific activity and kinetics of PodA. To determine specific activity and kinetic parameters of PodA for PYO, a continuous spectrophotometric assay monitoring the absorbance of PYO (690 nm) was utilized. Briefly, assays were performed at 25° C. in 100 μL reaction volumes in 96-well plates and reactions were monitored at 690 nm over 10 min. Reaction mixtures contained Phosphate buffer (50 mM, pH 6.0), ethylene glycol (1%, v/v), protein (3 μM) and substrate (for specific activity, 100 μM, for kinetics, varying). Data were acquired using the SpectraMax® M3 Microplate Reader (Molecular Devices) using the Soft Max Pro software every 10 s over 10 min. Path lengths for each well were calculated using Soft Max Pro endpoint readings and slopes were corrected for path lengths of 1 cm.

Specific activity was calculated from the slope of the linear range of (ΔOD₆₉₀ min⁻¹) using Beer's Law (A=εcl) with a path length of 1 cm and the molar extinction coefficient of PYO (4,130 M1 cm1). Equation was solved for c, giving specific activity in nmol min mg of PodA. For kinetic parameters, graphs of initial velocity (μM s1) versus substrate concentration (μM) were plotted using Prism v8 (GraphPad). The Michaelis-Menten kinetics model was used to determine K. and Vₘₐₓ. The turnover number (kₐₜ) was determined using the following equation: Vₘₐₓ=kₐₜ [E], where [E] was the concentration of PodA added. All spectrophotometric assays mentioned above were completed thrice, each in technical triplicate with a representative data set shown. Error bars represent standard deviation as calculated by Prism v8 (GraphPad). Standard deviation for k_{cat} and k_{cat} K_{M}⁻ was calculated using standard formulas for propagation of error.

Pseudomonas aeruginosa PodA and tobramycin liquid synergy experiments. For planktonic synergy experiments, starter cultures were grown 24 h at 37° C. shaking in LB. Stationary phase cultures (usually between OD500 4-6) were aliquoted (100 uL) into 96-well clear bottom flat well plates (VWR). Antibiotics and PodA were diluted into stock concentrations to pipette 5 μL antibiotic and 2 μL PodA per well. Antibiotics were diluted into water and PodA was diluted into 50 mM HEPES (pH 7.5). Plates were incubated in a humidity Tupperware chamber lined with wet paper towel at 37° C. for 10 h. After incubation, wells were pipetted to resuspend, and each well was diluted 1:10 into fresh LB. From that mixture, 10 μL of diluted cells were pipetted into 90 μL fresh LB in 96-well flat bottom plates. Mineral Oil, light (65 μL undiluted, Sigma-Aldrich) was pipetted onto top of cultures to prevent dehydration and allow for oxygen diffusion. Microtiter plates were incubated at 37° C. inside a temperature-controlled chamber of a BioTek Synergy 4 plate reader. Plates were continuously shaken (medium setting) and time points (OD₅₀₀) were taken for 24 h every 30 min. Data were plotted using Prism v8. Growth studies were completed thrice, with a representation from one experiment shown. For concentrations that led to an increase in lag times, the same experiment was set up but rather than an outgrowth step, cells were diluted over a dilution series and seven dilutions (10 μL each) were plated on LB agar to determine CFUs mL-1. Cells were plated in biological triplicate to obtain an average number of CFUs mL-1 to calculate a standard deviation. Cells from each 10 μL drips dilution were counted if colonies were between 10-100.

PodA 10 and tobramycin cell treatments (same samples from FIG. 8) were diluted in series and dilutions were plated onto LB agar. Cells were counted if a dilution contained 10-100 colonies.

Crystal violet assay. Pseudomonas aeruginosa grown overnight in LB were inoculated (1.5%, v/v) and grown for 24 h at 37° C. in 96-well round bottom plates in minimal medium (100 μL) with arginine (40 mM) as the sole carbon source. PodA was added with cells simultaneously and was present during growth and biofilm development. Planktonic cells were removed and attached cells were quantified with crystal violet staining (125 μL. 0.1% w/v), followed by washes with water and de-staining with an acetic acid (30%, v/v) mixture in water. Crystal violet was measured using a SpectraMax® M3 Microplate Reader (Molecular Devices) at an absorbance of 550 nm. Details of procedure were published previously (28) [43].

Pseudomonas aeruginosa PodA and tobramycin agar block biofilm assay (ABBA) synergy experiments. Starter Pseudomonas aeruginosa cultures were grown 24 h at 37° C. shaking in LB and the OD500 was determined by diluting cells 1:10. For inoculation into agar, cells were first diluted to an OD of 1.0, followed by a 1:10 dilution for a final OD of 0.1. Molten L B agar (1% w/v) or SCFM agar (1% w/v, made by mixing premade 2× SCFM and 2% agar) was incubated at 44° C., after which 10 μL of OD 0.1 culture was added and mixed into 1 mL of agar, for a final starting OD of 0.001. A portion of the mixture (200 μL) was pipetted into a well of an 8-well glass chamber slide for microscopy (Thermo Fisher Scientific #155409) or into a 2 mL Eppendorf tube for cell viability experiments. ABBAs were incubated at 37° C. for 24 h in a Tupperware lined with wet paper towels. Following growth, ABBAs were washed with 200 μL of HEPES (20 mM, pH 7) to rid planktonic cells growing on the surface of the agar. Treatment stocks (100 μL each) were made by diluting PodA 10 and/or tobramycin into HEPES (final concentration of 20 mM, pH 7), with the no treatment control consisting of solely HEPES. Final concentrations of PodA (5 μM) and tobramycin (50 ug mL-1) were calculated accounting for the total volume of the agar+liquid. ABBAs were treated overnight (14 h), treatment mixture was decanted, and agar blocks were washed with HEPES (400 μL).

For microscopy experiments, cells were incubated for 30 min with 125 μL of diluted propidium iodide (final concentration of 490 UM when added to ABBA, taking into account volume of the agar) and SYTO-9 (final concentration of 16 μM), after which 50 μL of a 1:50 dilution of 5-μm fluorescent beads (Spherotech; CFP-5045-2) was added to each agar block. ABBAs were imaged with a Leica TCS SPE confocal microscope as described elsewhere using 512×512-pixel format (29) [44], with the exception of imaging with a 6 μm step-size in the Z-direction.

For cell viability experiments, 300 μL of HEPES (20 mM, pH 7) was pipetted into each Eppendorf ABBA tube and samples were homogenized on speed 3 of a Bio-Gen PRO200 Homogenizer (PRO Scientific). Samples were briefly spun to rid of air bubbles (<1,000×g) and diluted in series and plated as described above in liquid synergy experiments. Experiments were completed with biological triplicates and was repeated thrice, with a representative figure shown.

ABBA microscopy image analysis. Images were analyzed in FIJI by re-slicing the data set in the XZ dimension followed by a max-intensity Z-projection. The brightness/contrast was auto-normalized in FIJI to the sample treated with only tobramycin. These LUT values were then applied to the other images in the corresponding data sets. Average pixel intensities along the Z-dimensions were found using the “Plot Profile” function in FIJI. Experiments were completed in a minimum of biological triplicate with a representative replicate shown in figures.

To determine mean pixel intensity and aggregate volume of each ABBA, channels 1 (SYTO-9) and 2 (Propidium Iodide) were combined into a single channel using FIJI. Images were analyzed in Imaris and aggregate masks were created using the “surface” module of the software. Using the surface Imaris wizard, all options under algorithm settings were unchecked. For surface channel settings, channel 3 (representing combined channels) was chosen and default settings were maintained. For threshold settings, the no treatment ABBA sample was analyzed for proper data inclusion and a value of 60 was used for all LB samples, with a threshold value of 40 for all SCFM samples. Lastly, data were excluded 0.1 μm from the XY edges, to exclude aggregates that were cut off due to imaging field. Data were not excluded from the top edge of the ABBA, as images were collected from the surface of the agar using fluorescent beads as described above. Exported statistics were used to plot aggregate volume or mean PI intensity against position of aggregates in the Z-dimension.

To determine mean and 95% confidence intervals of PI staining of ABBAs, collections of aggregates were binned by 25 μm through the depth of the agar. Then 10,000 bootstrap replicates per bin were produced, took the mean of each of those replicates, then calculated the 2.5 and 97.5 percentiles of those collections to calculate the 95% confidence interval.

Oxygen probe measurements. ABBAs were inoculated and set-up identically as in the section titled “Pseudomonas aeruginosa PodA and tobramycin agar block biofilm assay (ABBA) synergy experiments.” ABBAs were incubated for 24 h at 37° C. in a humidity chamber and were incubated in a 37° C. sand bath during oxygen probe measurements. Oxygen concentrations were measured using a Clark-type amperometric electrode with a 10 μm tip diameter, which was connected to an amplifier of a multimeter (Unisense, Denmark). A two-step calibration was performed using an oxygen free solution (0.1 M NaOH, 0.1 M sodium ascorbate) and an oxygen saturated LB solution (with 1% w/v salinity). The agar surface was found by decreasing the tip depth by 25 μm until oxygen measurements declined by 2 μmol L⁻¹. The oxygen sensor was positioned 100 μm above the agar interface, and data were collected in 25 μm steps for a total of 700 μm. Technical triplicate measurements were made at each depth, with each measurement taking 3 s, with 2 s between measurements. Oxygen profile data are averages from one experiment of technical triplicates of biological triplicates, with each experiment done in triplicate on different days. Data were collected using SensorTrace Pro 3.1.3 software and were plotted using Prism v8 (GraphPad).

### Example 1. Characterization of PodA Activity and Kinetics

To assess PodA's activity of therapeutic relevance in the context of human chronic infections, PodA activity was quantified and its kinetic parameters determined under a spectrum of physiologically relevant variables (i.e. pH, viscosity, sodium and potassium concentrations).

As done previously to facilitate PodA purification (12) [1], a predicted N-terminal transmembrane domain was excluded, resulting in a wild type variant called WT PodA₄₁₋₁₆₂.

In particular, PodA₄₁₋₁₆₂ is a truncated version the protein encoded by MFORT_14352 (lacking a predicted N-terminal, membrane-spanning helix), The gene expressing PodA₄₁₋₁₆₂ derived from Mycobacterium was heterologously expressed in E. coli. from Escherichia coli. In particular, PodA₄₁₋₁₆₂ in the sense of the disclosure can have sequence (SEQ ID NO: 3)

Moving forward in the present and subsequent examples, all PodA purified proteins maintained this deletion.

WT PodA₄₁₋₁₆₂ demethylation activity was measured and compared with active site variants, using a spectrophotometric assay that monitors the absorbance of PYO over time

In particular the specific activity of WT PodA₄₁₋₁₆₂ under varying conditions. WT PodA₄₁₋₁₆₂ (3 μM) was incubated with PYO (100 μM) and activity was monitored by measuring absorbance of PYO over time. Activity was analyzed by changing pH, viscosity, or salt concentrations. The results are shown in FIG. 5.

Using a variety of buffers to analyze activity from pH 5.5-10.0, it was found that WT PodA₄₁₋₁₆₂ had optimal activity at pH 6 as shown in the illustration of FIG. 5A.

Activity of WT PodA₄₁₋₁₆₂ below pH 5.5 was not established due to the pKₐ of PYO (4.9), which changes the chemical and therefore absorptive properties of the substrate. Using a reaction condition with a pH of 6, a slight increase in viscosity with ethylene glycol or glycerol (1% v/v) enhanced WT PodA₄₁₋₁₆₂ activity (FIG. 5A).

In the case of glycerol, higher concentrations (5%-20%) reverted this activity increase.

Average ion concentrations of potassium and sodium in cystic fibrosis sputum are 15 mM (K⁺) and 66 mM (Na⁺) (15) [45]. Addition of these ions in salt form (NaCl and KCl) in this concentration range did not inhibit or enhance WT PodA₄₁₋₁₆₂ activity (FIG. 5A).

For these reasons, conditions were established for kinetic analyses to be carried out at a pH of 6 with 1% ethylene glycol.

WT PodA₄₁₋₁₃₂ was also characterized in comparison with selected variants PodA^{D68A}, PodA^{D72A}, PodA^{H12IA}, PodA^{H12IK}, PodA^{HI2IR}, PodA^{E154A}, PodA^{Y156A}, and PodA^{Y156}F

In particular, WT PodA₄₁₋₁₃₂ or related variants (3 μM) and PYO (concentrations indicated on x-axis) were incubated and monitored at 690 nm. Vₘₐₓ values were plotted against [PYO] and an K was calculated by fitting data to the Michaelis-Menten equation

Using Michaelis-Menten parameters, WT PodA₄₁₋₁₆₂ exhibited a K_{M} of 0.372 mM±0.018, a k_{cat} of 0.176±0.003 s⁻¹, and a catalytic efficiency of 4.74±0.24×10² M⁻¹ s⁻¹ (FIG. 5B).

WT PodA₄₁₋₁₆₂ active site variants PodA^{D72A}, PodA^{H12IA}, PodA^{H12IK}, PodA^{HI21R}, PodA^{E154A}, PodA^{Y156A}, and PodA^{Y156}F did not have measurable activity compared to controls. A variant inhibiting product release, PodA^{D68A}, maintained similar Michaelis-Menten trends for concentrations of PYO under 100 μM, after which Vₘₐₓ leveled off (FIG. 5B).

### Example 2. Identification of PodA Variants with Mutated Trimer Interface

Although the characterization of WT PodA₄₁₋₁₆₂ reported win Example 1 confirmed the enzyme's activity under a broad range of conditions, it also revealed limitation in the related protein yields (typically ˜0.3 mg L⁻¹ of bacterial culture).

Furthermore, that PodA purification demands laborious refolding experiments argued against using protein-optimization methods that require high or even medium-throughput experimental screening such as in vitro evolution or deep mutational scanning.

Accordingly, protein design was applied to attempt to identify possible variants with increased stability with the aim of improving yield while maintaining or improving activity.

The AffiLib protein-design method and a related method, called AbLIFT, were used. AffiLib was shown to improve protein binding affinity by almost two orders of magnitude (24) AbLIFT, led to dramatic improvement in antibody stability, expressibility and binding affinity through the design of mutations in the variable domain's light-heavy chain obligatory interface (26) [46].

The AffiLib design approach is especially relevant for the design of small-to-large mutations, since it uses Rosetta atomistic design calculations to enumerate all of the combinations of mutations that are observed in a phylogenetic analysis of sequence homologs and are individually tolerated according to computational mutation scanning. Each combination of mutations is allowed to relax, including through backbone and rigid-body minimization to promote the introduction of small-to-large mutations.

AffiLib was applied to PodA to increase its yield during heterologous overexpression. Since PodA is a homotrimer, all modeling and design simulations used symmetric sequence, backbone and rigid-body sampling. Furthermore, each PodA monomer binds a structural Ca²⁺ ion and any change in amino acid identity or conformation at the ion-ligation sites were disallowed.

Since PodA forms symmetric homotrimers, residues were selected to improve the interactions within these obligatory interfaces as well as complex stability and expressibility.

In particular, because PodA forms symmetric homo-trimers, it was hypothesized that improving the interactions at these obligatory interfaces will similarly improve complex stability and expressibility.

In particular in view of the results of structural analysis of these obligatory interfaces. residues and positions in the interface were selected also based on the observation that the related sites may be under-packed due to the presence of three alanine amino acids.

The AffiLib design approach is especially relevant for the design of small-to-large mutations, since it uses Rosetta atomistic design calculations to enumerate all of the combinations of mutations that are observed in a phylogenetic analysis of sequence homologs and are individually tolerated according to computational mutation scanning. Each of the enumerated mutants is allowed to relax, including through backbone and rigid-body minimization, to enable the entire trimeric structure to accommodate the introduction of small-to-large mutations.

Because PodA is a homotrimer, all modeling and design simulations used symmetric sequence, backbone, and rigid-body sampling. Furthermore, because the available crystallographic structure of PodA shows that each monomer binds a Ca²⁺ ion (FIG. 12), any change in amino acid identity or conformation at the ion-ligation sites was disallowed.

The designs were ranked according to their computed energies, and the top 35 designs were visually inspected. From those a diverse set of 10 mutants that exhibited improved core packing or hydrogen-bonding interactions across the obligatory interfaces was chosen Induction studies showed that a majority of the PodA variants were shuttled into inclusion bodies. A protocol was developed reflected in the material and methods reported in this examples section to denature and purify WT PodA₃₀₋₁₆₂ from inclusion bodies, followed by a refolding procedure.

Residues and positions were selected in view of the results of the above analysis performed with respect to PodA₄₁₋₁₆₂ and tested in PodA₄₁₋₁₆₂. The selected residues and positions are reported in FIG. 1 panel A with numbering referred to PodA of SEQ ID NO: 1.

In particular the illustration of FIG. 1 panel A and through the examples of the disclosure, the residue numbers for the selected position are indicated with respect the full protein sequence of PodA SEQ ID NO: 1, as that is the norm within the field, even if the experiments reported in the Examples were performed with PodA₄₁₋₁₆₂.

Accordingly in the set of examples of the present disclosure
the residue indicated as A53 corresponds to residue A13 of SEQ ID NO: 3,
the residue indicated as 173 corresponds to residue 133, of SEQ ID NO: 3,
the residue indicated as T87 corresponds to residue A47 of SEQ ID NO: 3,
the residue indicated as M99 corresponds to residue M59 of SEQ ID NO: 3,
the residue indicated as A129 corresponds to residue A89 of SEQ ID NO: 3, and
the residue indicated as K141 corresponds to residue K101 of SEQ ID NO: 3
   as will be understood by a skilled person upon reading of the present disclosure.

In particular, positions corresponding to A53, 173, A87, T91, M99, A129 and K141 of PodA of SEQ ID NO: 1 were chosen (See FIG. 1 panel A where positions that were subjected to design are marked by spheres).

Mutations were designed and the resulting designs were ranked according to their computed energies and the top 35 designs were visually inspected.

From those, for experimental testing a diverse set of 10 mutants that exhibited improved packing or hydrogen-bonding interactions across the obligatory interfaces the variants reported in the following Table 7 and in FIG. 1 panels A-D were chosen.

TABLE 7

| | WT | | | | | | | | | | |
|---|---|---|---|---|---|---|---|---|---|---|---|
| Protein | PodA | PodA1 | PodA2 | PodA3 | PodA4 | PodA5 | PodA6 | PodA7 | PodA8 | PodA9 | PodA10 |
| Residue | (−) | I73K | I73T | I73K | I73E | I73L | I73R | I73L | A53N | I73K | A53N |
| Changes | | A87V | A87V | A87V | A87V | A87I | A87V | A87I | I73R | A87V | I73T |
| | | T91V | T91V | T91V | M99I | M99V | T91V | T91V | A87V | T91V | A87V |
| | | M99T | M99V | M99V | A129V | A129V | M99V | M99T | T91V | A129V | M99V |
| | | A129V | A129V | 129V | K141T | K141T | A129T | A129V | A129V | K141T | A129T |

In particular, testing indicated that mutations Ala87Val and A129V increase core packing in a hydrophobic region (see FIG. 1 panel B), mutations Ile73Thr, Ile73K, Ile73R, and Ile73E improves surface polarity (see FIG. 1 panel C), mutation Ala53Asn forms an interfacial hydrogen bond with a Ser (see FIG. 1 panel D) K14T contributes to the maintenance of a stable configuration.

### Example 3: Characterization of Yields in Production of PodA Variants in Comparison with WT PodA

Induction studies were performed by providing overexpression cultures of designed variants were analyzed using BugBuster™. The results illustrated by a representative SDS-PAGE gel for PodA1-PodA3 shown in FIG. 6 indicated that a majority of the PodA variants were shuttled into inclusion bodies (FIG. 6).

A protocol was developed (see Materials and Methods) to denature and purify WT PodA₄₁₋₁₆₂ from inclusion bodies, followed by a refolding procedure. Before moving forward using the inclusion body purification exclusively for the designed variants, it was verified that WT PodA₄₁₋₁₆₂ activity and kinetics were not altered after denaturing and refolding the enzyme. Soluble fraction WT PodA₄₁₋₁₆₂ had a catalytic efficiency of 1.05±0.093×10² M⁻¹ s⁻¹ and WT PodA₄₁₋₁₆₂ purified from inclusion bodies had a catalytic efficiency of 1.14±0.25×10² M⁻¹ s⁻¹.

Remarkably, eight PodA designs exhibited increased protein yields relative to the wild-type protein as shown by the yield reported in Table 8 and FIG. 1 panel E.

TABLE 8

| | WT | | | | | | | | | | |
|---|---|---|---|---|---|---|---|---|---|---|---|
| Protein | PodA | PodA1 | PodA2 | PodA3 | PodA4 | PodA5 | PodA6 | PodA7 | PodA8 | PodA9 | PodA10 |
| Yield | 0.33 | 0.20 | 16.1 | 9.45 | 0.38 | 9.9 | 4.4 | 4.1 | 2.1 | 1.28 | 5.92 |
| mg/L⁻¹ | | | | | | | | | | | |
| Residue | (−) | I73K | I73T | I73K | I73E | I73L | I73R | I73L | A53N | I73K | A53N |
| Changes | | A87V | A87V | A87V | A87V | A87I | A87V | A87I | I73R | A87V | I73T |
| | | T91V | 791V | T91V | M99I | M99V | T91V | T91V | A87V | T91V | A87V |
| | | M99T | M99V | M99V | A129V | A129V | M99V | M99T | T91V | A129V | M99V |
| | | A129V | A129V | 129V | K141T | K141T | A129T | A129V | A129V | K141T | A129T |

The above results are consistent with the expectation that the trimeric interfaces in the wild-type PodA were unstable and that improving packing at these interfaces might improve protein production yields.

In particular, the eight variants PodA2, PodA3, PodA5, PodA6, PodA7, PodA8, PodA9 and PodA10, showed a significant increase in yield with respect to WT PodA and the production yields of was successfully increased by optimizing contacts across the obligatory homotrimeric interfaces with selected positions and replacements.

In particular, variants PodA3, PodA5, PodA6, PodA7, and PodA10, showed an increase in yield of one order of magnitude with respect to the wild type.

In general the variants allow production yields of PodA 20-fold increased. This enabled the study of the physiological effects of PodA on planktonic and biofilm-grown P. aeruginosa cells under conditions relevant to human infections, revealing significant synergistic killing with the commonly used clinical antibiotic, tobramycin.

### Example 4. Characterization of PodA Variants Stability Activity and Kinetics in Comparison with WT PodA

The same continuous spectrophotometric assay used to characterize WT PodA₄₁₋₁₃₂ in Example 1 was used to determine enzyme kinetics of PodA and the variants identified in Example 2 for PYO.

The eight variants PodA2, PodA3, PodA5, PodA6, PodA7, PodA8, PodA9 and PodA10 were tested for kinetics and the results are reported in FIG. 2)

In particular, PYO (50 μM) demethylation activity of PodA variants (3 μM) was determined using a spectrophotometric assay described in the Materials and Methods. As can be seen by the illustration of FIG. 2 panel A, of the eight variants tested, wild-type activity was maintained for PodA8 and PodA10 (FIG. 2 panel A).

Kinetics of these proteins were compared. In particular Michaelis-Menten kinetics of PodA8 and PodA10 (3 μM) were found by plotting Vₘₐₓ values against [PYO], as indicated on x-axis of the chart shown in FIG. 2 panel B and it was found no change in catalytic efficiency between PodA^{WT} and PodA8 and a slight increase in efficiency for PodA10 (FIG. 2 panel B). Due to the slight increased efficiency combined with a higher yield, it was decided to utilize PodA10 for in studies of PodA effects on P. aeruginosa.

As in FIG. 5 panel A with WT PodA₄₁₋₁₆₂, PodA10 activity was analyzed across a broad range of buffers, salts, and viscosity agents.

In particular, PodA10 specific activity under varying conditions. PodA10 (3 μM) was incubated with PYO (100 μM) and activity was monitored by measuring absorbance of PYO over time.

Unlike WT PodA₄₁₋₁₆₂, PodA10 162 maintained activity over a large range of pH, with no observable preference between a pH of 5.5 and 9 (FIG. 1 panel D), indicating that the design was also more stable to environmental conditions than the wild-type enzyme. PodA 10 activity was not altered with addition of salts, and a slight ad-vantage was seen with addition of ethylene glycol (FIG. 2 panel D), similar to WT PodA₄₁₋₁₆₂.

Activity was analyzed by changing pH, viscosity, or salt concentrations. The results are shown in FIG. 2C

As shown in FIG. 2 panel C. PodA 10 maintained activity over a large range of pH, with no observable preference between a pH of 5-9 (FIG. 2 panel C). PodA10 activity was not altered with addition of salts, and a slight advantage was seen with addition of ethylene glycol (FIG. 2 panel C), similar to WT PodA₄₁₋₁₆₂.

Due to the high thermal stability of PodA, stability assays that rely on monitoring protein unfolding over a temperature gradient were not suitable for the related testing. Therefore the enzyme's kinetic stability were analyzed by boiling WT PodA₄₁₋₁₆₂ and PodA10 and measuring activity of each protein after specified boiling times. After 30 and 60 min of boiling, PodA10 had significantly higher activity compared to WT PodA₃₀₋₁₆₂ (FIG. 13). Thus, the optimized inter-subunit interactions in the designed PodA10 resulted in significantly higher Escherichia coli expression levels and kinetic stability compared to the parental enzyme. We also noted that several designed mutations in PodA8 and PodA10 were not prevalent in a multiple-sequence alignment of PodA homologs (FIG. 14), indicating the importance of the atomistic calculations to design success

Even if activity of PodA2, PodA3, PodA5, PodA6, PodA7, PodA8 and PodA9, have not been tested it is expected that in view of the higher stability during overexpression those variants will have a higher stability in pH, viscosity, and salinity. Accordingly, PodA3, PodA5, PodA6, PodA7, PodA8 and PodA9 are expected to have an activity

### Example 5. Addition of PodA10 to P. aeruginosa Cultures in Combination with Antibiotics Enhances Planktonic Killing and Blocks Biofilm Development

Prior to testing the impact of PodA10 on biofilms, it was sought to determine its effects on P. aeruginosa grown planktonically.

To ensure its activity and kinetics would not be compromised in physiologically relevant growth medium, PodA10 PYO demethylation activity was tested in lysogeny broth (LB) and Synthetic Cystic Fibrosis Medium (SCFM1, (15)) [45].

In particular, Using a continuous spectrophotometric assay as described in the Material and Methods, PodA (3 μM) and PYO (concentrations indicated on x-axis) were incubated and monitored at 690 nm. Vₘₐₓ values were plotted against [PYO] and an Ky was calculated by fitting data to the Michaelis-Menten equation.

The results are shown in FIG. 7. No change was observed in catalytic efficiency in either medium compared to the kinetic experiments performed in buffer (FIG. 7), allowing moving forward with studying PodA10 effects on P. aeruginosa.

Planktonic cultures of P. aeruginosa strain PA14 were therefore grown to high densities in LB or SCFM over a range of concentrations of PodA10 (0-20 μM) and tobramycin (0-100 μg mL⁻¹). Cells were incubated statically to reduce access to oxygen and require P. aeruginosa to utilize PYO to sustain viability.

After incubation, cells were resuspended and reinoculated into fresh medium. Lag times were calculated for each well and it was hypothesized those with increased lag times reflected increased cell death from the treatment.

The same experiments in SCFM were repeated, a minimal medium whose composition is based upon that of sputum from cystic fibrosis patients (15) [45] and which elicits a similar response from P. aeruginosa grown in vitro compared to the in vivo environment (27) [47].

The results of the experiments are shown in FIG. 3 panel A (LB), FIG. 3 panel B (SCFM) and FIG. 8 (LB and SCFM).

A review of the results indicates that while slight differences were seen in lag times when PodA10 was added with tobramycin in SCFM, the effects were not as dramatic as seen in our experiments with LB (FIG. 8).

However, when plating the same SCFM cultures to calculate viability via CFUs, there was a significant decrease in survival when PodA 10 (5 μM) was added with tobramycin (FIG. 3 panel B). It was speculated that the inconsistent lag times observed in our experiments (FIG. 8) were due to cells adjusting from being treated in SCFM and then grown in LB. This indicates that direct cell counts are a better test for analyzing PodA10 and tobramycin synergistic killing. Contrary to lag times, synergy between PodA and tobramycin in SCFM was clear from directly counting cells (FIG. 3 panel A) and that lag times were not a good universal analysis of synergy (FIG. 8).

Other variants were not tested, but it expected that if activity of a PodA variant is comparable PodA10, then the efficacy for ability to kill Pseudomonas will be similar.

### Example 6. Effects of PodA10 in Combination with Antibiotic on Planktonic Viability and Crude Biofilm Development

After it was established that PodA10 enhances killing of planktonic P. aeruginosa, experiments were performed to verify that the designed variant could also inhibit biofilm formation as shown previously for WT PodA₄₁₋₁₆₂ (12) [1].

In particular for the first biofilm test, the classic biofilm crystal violet microtiter plate assay was to measure the amount of biomass attached to microtiter plate wells after a defined period of time (28) [43]. Incubation for 24 h with addition of designed variant PodA10 from the beginning of the assay led to a biofilm attachment defect that was comparable to that achieved by WT PodA₄₁₋₁₆₂ (FIG. 3 panel C).

Similarly what indicated for Example 5, also in this case other variants were not tested, but it expected that if activity of a PodA variant is comparable PodA 10, then the efficacy for ability to kill Pseudomonas will be similar.

### Example 7. Addition of PodA10 to Aggregate Biofilms Together with Conventional Antibiotics Leads to Synergistic Killing

Based on the results of the experiments in Example 5 and Example 6, synergistic concentrations of PodA and tobramycin to be analyzed in triplicate were identified.

Prior to testing the im-pact of PodA10 on biofilms, we sought to determine its effects on P. aeruginosa grown planktonically. To ensure that its activity and kinetics would not be compromised in physiologically relevant growth medium, we tested PodA10 PYO demethylation activity in lysogeny broth (LB) and synthetic cystic fibrosis medium (SCFM). No change in catalytic efficiency was detected in either medium compared to experiments performed in buffer (FIG. 7), which allowed moving forward with studying PodA10 effects on P. aeruginosa.

Accordingly the next assessment was directed to verify whether PodA is synergistic with tobramycin (an antibiotic commonly used to treat P. aeruginosa infections in the clinic). Tobramycin is an aminoglycoside that targets the ribosome, and its efficacy requires a sufficient proton motive force for entry. The minimum inhibitory concentration (50%) for tobramycin on Pseudomonas ranges from 1 to 3 μg·mL⁻¹ (31), with the target tobramycin concentration in sputum of cystic fibrosis infections being ˜130 ug·mL⁻¹ [50].

To assess whether PodA10 is synergistic with tobramycin, planktonic cultures of P. aeruginosa strain PA14 were grown to high densities in LB or SCFM over a range of concentrations of PodA10 (0 to 20 μM) and tobramycin (0 to 100 ug·mL-1). Cells were incubated statically. After incubation, cells were resus-pended and reinoculated into fresh medium. Lag times were calculated for each well, and we hypothesize that increased lag times reflect increased cell death from the treatment. Based on the results of these screens, synergistic concentrations of PodA and tobramycin were identified to be analyzed in triplicate.

In particular, to complete preliminary experiments in triplicate, and perform targeted experiments in biological triplicate with technical triplicate in LB and SCFM, a minimum of 10 mg of PodA was needed. To perform these studies, instead of purifying protein from over 30 L of culture for WT PodA₄₁₋₁₆₂ but, due to the improvements in yield observed for PodA10, the goals were achieved using only 1.6 L of culture—a substantial improvement in experimental efficiency.

In LB, addition of PodA10 (>1 μM) together with tobramycin increased lag times compared to PodA (−) controls (FIG. 8).

When PodA10 was added with tobramycin, lag times increased by 1.5-2.0 h compared to no PodA controls (FIG. 8)

To verify that lag times were due to increased cell death, in the same experimental set up, cells were plated for colony forming units (CFUs). PodA10 (5 μM) alone did not increase cell death, but when PodA10 was added with tobramycin, cell viability greatly decreased compared to the individual antibiotic controls (FIG. 3 panel A).

The same experiments were repeated in SCFM, a minimal medium whose composition is based upon that of sputum from cystic fibrosis patients and which elicits a similar response from P. aeruginosa grown in vitro compared to the in vivo CF lung environment [47]. While slight differences were seen in lag times when PodA10 was added with tobramycin, the effects were not as dramatic as seen in our experiments with LB (FIG. 8). However, when plating the same SCFM cultures to calculate viability via CFUs, there was a significant decrease in survival when PodA10 (5 μM) was added with tobramycin com-pared to no protein or inactive PodA10 controls (FIG. 3 panel B). It is expected that the inconsistent lag times observed in our experiments (FIG. 8) were due to cells adjusting from being treated in minimal defined medium (SCFM) and then grown in rich medium (LB), an observation that is common in microbiological growth experiments. This indicates that direct cell counts are a better test for analyzing PodA10 and tobramycin synergistic killing. impacts mature biofilms and their viability.

In planning additional experiments, it was noted that while the crystal violet assay permits biofilm phenotypes to be rapidly screened, it only provides a crude measurement of a biofilm defect related to attachment. To better understand how PodA10 impacts biofilm development and longevity, the agar block biofilm assay (ABBA) was used, which was designed to enable the experimental study (12, 29) of the type of aggregate biofilms that characterize human chronic infections (30) [51].

Due to the ABBA assay being less high throughput than experiments in liquid cultures, it was chosen to test a single concentration of tobramycin (50 ug mL-1) with PodA10 (5 μM) in LB and SCFM, as these concentrations showed the most dramatic synergistic effects in previous experiments (FIG. 3, FIG. 8).

Previous ABBA experiments performed in succinate-minimal medium with WT PodA+1-162 showed that addition of PodA led to smaller aggregates in anoxic regions of the agar after 27 hours of incubation (12) [1].

These experiments also showed that inactive PodA₄₁₋₁₆₂ had no effect on P. aeruginosa aggregate development. Given this, and the low protein yield for expression of this inactive PodA variant (which was not redesigned with AffiLib), it was opted to use buffer as a negative control for these experiments.

Cells were inoculated into the agar and allowed to grow into aggregates for 24 hours, after which PodA10 and tobramycin were added simultaneously and allowed to incubate for 12 hours (FIG. 4A). Samples were then stained with propidium iodide (PI), a fluorescent DNA binding dye that is incapable of passing through the membrane of viable cells, and imaged. Generally, higher PI staining indicates lower cell viability due to the compromised membranes of these cells. Aggregates were analyzed via confocal microscopy throughout the depth of the ABBA.

Addition of PodA10 and tobramycin for LB and SCFM was administered at the latest time point for which an oxygen gradient was present (24 h for LB: 12 h for SCFM) (FIG. 17). This ensured that biofilms were at their most mature state, while still exhibiting metabolic stratification. After treatment, samples were stained with propidium iodide (PI), a fluorescent DNA-binding dye that is incapable of passing through the membrane of highly energized cells and imaged. Generally. higher PI staining indicates lower cell viability due to the com-promised membranes of these cells.

Aggregates were analyzed viaconfocal microscopy throughout the depth of the ABBA, with each aggregate representing a distinctly growing Pseudomonas biofilm. Incubation of LB-grown aggregates with tobramycin and PodA10 led to PI staining intensity that was significantly greaterthan PodA10 or tobramycin alone (FIG. 4B).

In particular Incubation of LB-grown aggregates with tobramycin and PodA10 led to a PI staining intensity that was visibly higher than PodA10 or tobramycin alone (FIG. 4B).

To quantify this visual effect, the average fluorescence intensity of PI in each sample was measured through the XZ-plane of the ABBA (FIG. 4C). In LB, the peak PI intensity with PodA10 and tobramycin was higher than the peak intensity of tobramycin alone (193.3 AUs vs. 151.0 AUS, respectively). To ensure higher intensities were not due to differing aggregate sizes, the average volume of each aggregate was plotted over depth and saw no significant differences in volumes between treatments (FIG. 9).

Plotting the aggregate volume vs. PI intensity of each aggregate further showed that PI staining was highest in the PodA10+tobramycin treated samples (FIG. 9). These staining results correlated with cell viability, which was determined by homogenizing ABBAs and plating for CFUs. ABBAs that were incubated with PodA10 and tobramycin had the lowest viable cell counts compared to controls (FIG. 4D).

For the LB ABBA, the largest differences in PI staining between tobramycin and tobramycin+PodAll (FIGS. 4C and 4F) were between 0 and 300 μm, a region previously characterized as the oxic-hypoxic zone within the agar (12) [1]. To confirm this, parallel experiments were set up identically and microelectrodes were used to measure the oxygen profiles in our ABBA system.

An oxygen gradient existed through the depth of the LB ABBA and correlated with the viability patterns observed (FIG. 4C, FIG. 17). Importantly, the oxygen gradient changes over time: at 12 hours, no oxygen is present at a depth of ˜350 um (FIG. 10, FIG. 17); however, by 24 hours, oxygen is again detected. These dynamics likely a change in what is limiting metabolism at these different timepoints: first oxygen, and then carbon. The expectation which is not intended to be limiting is that these dynamics reflect a change in the limiting nutrient at these different time points: first oxygen, then carbon. Once carbon is depleted, oxygen concentrations rise againdue to diffusion and a lack of cellular consumption.

At smaller scales, an oxygen gradient also exists within each single aggregate, due to cellular oxygen consumption outpacing its diffusion (31) [52]. Additionally, gradients of phenazines have been shown to exist within biofilms, with PYO being localized to the periphery (32), reflecting its requirement for molecular oxygen for its biosynthesis. Analyzing data from FIG. 4B in a XY-plane (top-view) rather than a XZ-plane (side view) a staining pattern consistent with such gradients was found: untreated aggregates grown in LB show an outside ring PI staining pattern that is abolished upon PodA addition (FIG. 11 and FIG. 16).

Similar synergistic effects of PodA and tobramycin treatment were observed for biofilm aggregates grown in SCFM. Qualitative differences in PI staining trends were also seen in this medium (FIG. 4E), with the peak PI intensity of PodA10 with tobramycin being higher than tobramycin alone (31.2 AUs vs. 22.6 AUs, respectively) (FIG. 4F). This difference was lower compared to that seen in LB, as reflected in the viability counts (FIG. 4G). While the differences were not as dramatic in SCFM as in LB, this was expected given that SCFM aggregates are smaller in size and therefore exhibit lower PI uptake (FIG. 9). In microelectrode experiments in 24 h grown SCFM ABBAs, no significant oxygen gradient decline was found, mirroring the higher PI staining intensity throughout the entire depths of the ABBA in the presence of tobramycin (FIG. 4F).

It is expected that antibiotics effective against phenazine producing bacteria will have comparable effect comparable to the one illustrated in this example. In particular a comparable efficacy is expected when PodA variant herein described are combined with one or more aminoglycosides (same antibiotic class as tobramycin).

### Example 8. Effects of Concentration on PodA Ability to Perform Synergistic Killing with Tobramycin and Other Antibiotics

PodA and antibiotics will be combined and applied to Pseudomonas cultures and cell viability will be calculated by counting alive cells after treatments. Treatments that have the highest killing compared to antibiotic or PodA only controls will be used for future PodA applications.

In order to determine synergistic effects of PodA and antibiotics, concentrations of PodA or engineered PodA can be tested at 0.01 μM, 0.1 μM, 1.0 μM, 5.0 μM, 10.0 μM, and 20.0 μM.

For antibiotics, concentrations are tested ranging from 100× below the MIC to 10× above the MIC. To determine synergy all possible combinations of PodA and tobramycin can be combined with a dense culture of bacterial cells and incubated for 12 hours.

Afterwards, cell viability can be determined by diluting cells and plating to calculate colony forming units.

Any combinations that lead to further killing compared to the sum of either compound alone, will be considered synergistic. Combinations of PodA and antibiotics that are equal to the sum or less of each compound alone, are considered non-synergistic.

Specific PodA concentrations that were tested were: 0.001 μM, 0.01 μM, 0.1 μM, 1.0 μM, 5.0 μM, 10.0 μM, and 20 μM.

Concentrations of the exemplary antibiotics tobramycin tested: 0.00005, 0.002, 0.01, 0.025, 0.08. 0.1. 2.0, 10.0, 25.0, 50.0, and 100.0 ug mL-1.

The concentration indicated in bold were shown to have synergic effect and are known or expected to be able to provide synergic effects in any combinations one with another.

The PodA concentrations were tested on PodA1041-162, are expected to be effective for PodA1032-162. PodA10 wt and also for the other variants exemplified herein as well as for PodA wild type and any other PodA derivatives as will be understood by a skilled person upon reading of the present disclosure.

### Example 9. PodA₃₀₋₁₆₂ Activity Is Stable Across Conditions Relevant to Cystic Fibrosis Sputum in a manner equivalent to PodA₄₁₋₁₆₂

To assess PodA's potential for therapeutic relevance in the context of human chronic infections, such as those in the cystic fibrosis (CF) airways, PodA activity was quantified and its kinetic parameters determined under a spectrum of physiologically relevant variables (i.e., pH, viscosity, and sodium and potassium concentrations). As done previously to facilitate PodA purification [1]. A predicted N-terminal transmembrane domain, resulting in a wild-type (WT) variant called WT PodA₃₀₋₁₆₂ which includes the structure of PodA₄₁₋₁₆₂.

Experiments performed proved that PodA₃₀₋₁₆₂ variants had activity and behavior substantially identical to the variants of PodA #1 162 whose activity has been tested with experiments reported in the previous examples.

Using a variety of buffers to analyze activity from pH 5.5 to 10.0, we found that WT PodA₃₀₋₁₆₂ had optimal activity at pH 6. Activity of WT PodA₃₀₋₁₆₂ below pH 5.5 was not established due to the pKₐ of PYO (pKₐ=4.9), which changes the chemical and therefore absorptive proper-ties of the substrate. The pH of CF airway surface liquid is acidic and is thought to increase viscosity of CF mucus by influencing mucin electrostatic interactions [54]. Using a reaction condition with a pH of 6, a slight increase in viscosity with ethylene glycol or glycerol (1% vol/vol) enhanced WT PodA₃₀₋₁₆₂ activity.

In the case of glycerol, higher concentrations (5 to 20%) reversed this activity increase. Average ion concentrations of potassium and sodium in cystic fibrosis sputum are 66 mM (Na⁺) and 15 mM (K+) (16). Addition of these ions in salt form (NaCl and KCl) in this concentration range did not inhibit or enhance WT PodA3_{0 16}? activity For these reasons, we established conditions for kinetic analyses to be carried out at a pH of 6 with 1% ethylene glycol.

The same continuous spectrophotometric assay was used to determine enzyme kinetics of PodA for PYO. Using Michaelis-Menten parameters, WT PodA₃₀₋₁₆₂ exhibited a K_{M} of 0.372 mM±0.018, a k_{cat} of 0.176±0.003 s⁻¹, and a catalytic efficiency of 4.73±0.24×10² M⁻¹ s⁻¹. The WT PodA₃₀₋₁₆₂ active-site variants PodA^{D72A}, PodA^{HI21A}, PodA^{HI21K}, PodA^{HI2IR}, PodA^{E154A}, PodA^{Y156A}, and PodA^{Y156}F did not have measurable activity compared to controls, as previously seen [1]. A variant inhibiting product release (13), PodAD68A, maintained similar Michaelis-Menten trends for concentrations of PYO under 100 μM, after which Vₘₐₓ leveled off.

Therefore similarly to what indicated for PodAn 162 also for PodA₃₀₋₁₆₂ Stabilizing the PodA Trimer Interface Increased the yield by 20-Fold.

Before moving forward using the inclusion body purification exclusively for the designed variants, we verified that WT PodA₃₀₋₁₆₂ activity and kinetics were not altered after denaturing and refolding the enzyme. Soluble fraction WT PodA₃₀₋₁₆₂ had a catalytic efficiency of 1.05±0.093×10² M⁻¹·s⁻¹ and WT PodA₃₀₋₁₆₂ purified from inclusion bodies of the same prep had a catalytic efficiency of 1.14±0.25×10² M⁻¹·s⁻¹.

Remarkably, also for PodA₃₀₋₁₆₂ the eight PodA designs discussed in the preceding example with reference to PodA₄₁₋₁₆₂ exhibited increased protein yields relative to the wild-type protein when purified from inclusion bodies. Also for PodA₃₀₋₁₆₂ there is no change in the kinetics of these proteins and in catalytic efficiency between PodA^{WT} and PodA8₃₀₋₁₆₂ and a slight increase in efficiency for PodA10₃₀₋₁₆₂ substantially the same of the ones shown for the variant of PodA₄₁₋₁₆₂. Similar considerations apply to the results reporting yields and stability as well as planktonic killing and synergic results confirming the key importance of the positions and the replacements to obtain increased stability, activity and yield for stabilized PodA and related derivatives.

### Example 10. Stabilized PodA Variants are Expected to be Effective Therapeutics Alone or Preferably in Combination with Antibiotics

The phenazine PYO is beneficial to P. aeruginosa biofilm development and metabolic activity (13, 14) [55, 56], and cells that cannot synthesize phenazines have lowered rates of anoxic survival without added terminal electron acceptors (8, 33) [55, 57]. Phenazines are in an oxidized form when acting as electron acceptors, so it was wondered whether the conversion of PYOₒₓ to 1-OH—PHZ_{red} via PodA would be detrimental to P. aeruginosa metabolic vitality within the anoxic zone. Because oxygen was present throughout our SCFM samples, only such an effect in LB-grown ABBA aggregates was looked for.

Because PYO and other phenazines permit P. aeruginosa to adopt metabolic strategies that lead to evasion of antibiotic treatments (9-11) [60], it stands to reason that removal of these metabolites is expected to offer an attractive new therapeutic approach.

A review of the results provided in the preceding examples indicated that, PodA addition alone did not significantly change PI staining at depth (>300 μm in LB, where the anoxic zone begins after 12 hours). Three possible explanations for this phenotype which are not intended to be limiting comprise: 1) conversion of PYOₒₓ to 1-OH-PHZred still permits sufficient metabolic activity to persist over the time period of our assay (12 hours between PodA addition and PI staining) because 1-OH-PHZred can be oxidized and recycled; 2) given the low energetic state previously measured for P. aeruginosa surviving anaerobically by cycling phenazines (6), the membrane potential of cells at this depth of the ABBA system is sufficiently low that PI can be taken up as efficiently by metabolically active cells as dead cells, thus obscuring a viability readout; and/or 3) PYO is not the primary phenazine sustaining metabolic activity in the anoxic zones—rather, another phenazine, such as phenazine carboxamide (which can also sustain anaerobic survival (34) and localizes to the interior of large anoxic colony biofilms grown on 1% tryptone (32) [53]), may maintain viability under these conditions.

In contrast to the anoxic zone in LB, PodA addition alone or in combination with tobramycin produced striking effects in the oxic/hypoxic zones of the ABBA assay in both LB and SCFM (FIGS. 4A-4G). Importantly, while PYO is beneficial to cells when they are electron-donor replete but oxidant-limited, PYO is toxic to P. aeruginosa under regimes where electron donors are limited but oxidants are replete, such as in the oxic/hypoxic zone, where PYO can react with oxygen and cause oxidative stress (35) [62].

This fact, combined with the knowledge that PYO is maximally concentrated in the outer regions of large colony biofilms grown on 1% tryptone (32), supports the conclusion that for untreated cells in LB, PYO causes localized toxicity at the oxic interface of biofilm aggregates. Addition of PodA alone removes PYO from oxic regions, leading to greater metabolic activity of aggregates in these zones, as well as in the outer rings of large aggregates (reflected by lower PI staining).

In contrast, when PodA is added together with tobramycin, this enhancement of metabolic activity is a double-edged sword. Greater metabolic activity—specifically, having an inner membrane that is sufficiently energized to be able to take up tobramycin—has been shown to be correlated with greater tobramycin susceptibility (36) [48]. Accordingly, in ABBA regions that have both oxygen and PYO, the depletion of PYO by PodA sensitizes these cells to tobramycin treatment. Supportive of this hypothesis is the fact that SCFM ABBA was completely oxic and PI staining patterns of tobramycin or tobramycin+PodA 10 treated samples were considerably higher than no treatment controls at all depths (FIG. 4F). In the LB ABBA, the switch to hypoxia/anoxia occurred around 300 μm leading to metabolic restriction, correlating with no significant differences in PI staining between non-treated and tobramycin-treated samples (FIG. 4C).

It is expected that the differences in effects observed for PodA treatment (in the presence or absence of tobramycin) in LB and SCFM reflect differences in media composition, with varying electron donor: electron acceptor abundance. Indeed, SCFM has 3 mM glucose and 9 mM lactate as a carbon source, in contrast to LB which only has amino acids as carbon sources (37) [63]—it is therefore possible that in the oxic zone, cells in SCFM are more protected from PYO toxicity due to a high electron donor: electron acceptor ratio (35) [62]. Moreover, metabolic flux through the TCA-cycle is known to promote antibiotic susceptibility due to proton-motive force production, which subsequently facilitates the import of tobramycin (38) [49]. It is possible that the presence of sugars in SCFM alters metabolic flux and PMF production in P. aeruginosa in such a way that, relative to LB, cells are generally less sensitive to tobramycin. Finally, the ratios and production of different phenazines (e.g. PYO, PCA, PCN) in P. aeruginosa are known to vary depending on the carbon source in the growth medium (39) [64]; such variation could also explain the differences that was observed in PodA effects between our media.

Overall, the experiments demonstrates that a combined use of stabilized variants of PodA with tobramycin results in an enhancement of synergistic killing by a designed PodA and the antibiotic.

In this respect, the results obtained for PodA10 are expected to be representative of the activity of additional stabilized PodAs and that the activity can be regulated by modulating PodA concentration to be administered.

Accordingly, the results of the experiments provided in the instant application support the conclusion that stabilized PodA's having increased yield over WT PodA can be used as a biologic therapeutic for treating chronic P. aeruginosa infections a phenazine containing bacteria in combination with antibiotics such as tobramycin.

In summary, provided herein are engineered pyocyanin demethylases having replacements in in positions A53, 173, A87, T91, M99, A129 and K141 of pyocyanin demethylase PodA of SEQ ID NO: 1 or a derivative thereof and related phenazine degrading agents, compositions methods and systems. Preferred embodiments comprise PodA₃₀₋₁₆₂ (SEQ ID NO: 2) and more preferably PodA₄₁₋₁₆₂ (SEQ ID NO: 3) having replacements in positions corresponding to A53, 173, A87, T91, M99, A129 and K14 of SEQ ID NO: 1 as will be understood by a skilled person upon reading of the disclosure, or a derivative thereof. Also provided herein is a combined administration of one or more pyocyanin demethylases and antibiotics and/or antibiotics resulting in a synergic inhibition of viability of phenazine producing bacteria, and related phenazine degrading agents, compositions methods and systems.

The examples set forth above are provided to give those of ordinary skill in the art a complete disclosure and description of how to make and use the embodiments of the compounds, compositions, systems and methods of the disclosure, and are not intended to limit the scope of what the inventors regard as their disclosure. All patents and publications mentioned in the specification are indicative of the levels of skill of those skilled in the art to which the disclosure pertains.

The entire disclosure of each document cited (including patents, patent applications, journal articles, abstracts, laboratory manuals, books, or other disclosures) in the Background, Summary, Detailed Description, and Examples is hereby incorporated herein by reference. All references cited in this disclosure are incorporated by reference to the same extent as if each reference had been incorporated by reference in its entirety individually. However, if any inconsistency arises between a cited reference and the present disclosure, the present disclosure takes precedence.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the disclosure has been specifically disclosed by preferred embodiments, exemplary embodiments and optional features, modification and variation of the concepts herein disclosed can be resorted to by those skilled in the art upon the reading of the present disclosure, and that such modifications and variations are considered to be within the scope of this disclosure as defined by the appended claims.

It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

When a Markush group or other grouping is used herein, all individual members of the group and all combinations and possible subcombinations of the group are intended to be individually included in the disclosure. Every combination of components or materials described or exemplified herein can be used to practice the disclosure, unless otherwise stated. One of ordinary skill in the art will appreciate that methods, device elements, and materials other than those specifically exemplified can be employed in the practice of the disclosure without resort to undue experimentation. All art-known functional equivalents, of any such methods, device elements, and materials are intended to be included in this disclosure. Whenever a range is given in the specification, for example, a temperature range, a frequency range, a time range, or a composition range, all intermediate ranges and all sub-ranges, as well as, all individual values included in the ranges given are intended to be included in the disclosure. Any one or more individual members of a range or group disclosed herein can be excluded from a claim of this disclosure. The disclosure illustratively described herein suitably can be practiced in the absence of any element or elements, limitation or limitations which are not specifically disclosed herein.

A number of embodiments of the disclosure have been described. The specific embodiments provided herein are examples of useful embodiments of the disclosure and it will be apparent to one skilled in the art that the disclosure can be carried out using a large number of variations of the devices, device components, methods steps set forth in the present description. As will be obvious to one of skill in the art, methods and devices useful for the present methods can include a large number of optional composition and processing elements and steps.

In particular, it will be understood that various modifications may be made without departing from the spirit and scope of the present disclosure. Accordingly, other embodiments are within the scope of the following claims.

### REFERENCES

1. Costa, K. C., et al., Pyocyanin degradation by a tautomerizing demethylase inhibits Pseudomonas aeruginosa biofilms. Science, 2017. 355 (6321): p. 170-173.
2. Thomsen, M. C. F. and M. Nielsen, Seq2Logo: a method for construction and visualization of amino acid binding motifs and sequence profiles including sequence weighting, pseudo counts and two-sided representation of amino acid enrichment and depletion. Nucleic acids research, 2012. 40 (W1): p. W281-W287.
3. Mentel, M., et al., Of two make one: the biosynthesis of phenazines. ChemBioChem, 2009. 10 (14): p. 2295-2304.
4. Pierson, L. S. and E. A. Pierson, Metabolism and function of phenazines in bacteria: impacts on the behavior of bacteria in the environment and biotechnological processes. Applied microbiology and biotechnology, 2010. 86 (6): p. 1659-1670.
5. Turner, J. M. and A. J. Messenger, Occurrence, biochemistry and physiology of phenazine pigment production. Advances in microbial physiology, 1986. 27: p. 211-275.
6. Gallagher, L. A., et al., Functions required for extracellular quinolone signaling by Pseudomonas aeruginosa. Journal of bacteriology, 2002. 184 (23): p. 6472-6480.
7. Costa, K. C., et al., Enzymatic degradation of phenazines can generate energy and protect sensitive organisms from toxicity. MBio, 2015. 6 (6): p. e01520-15.
8. Altschul, S. F., et al., Gapped BLAST and PSI-BLAST: a new generation of protein database search programs. Nucleic acids research, 1997. 25 (17): p. 3389-3402.
9. Smith, T. F. and M. S. Waterman, Identification of common molecular subsequences. Journal of molecular biology, 1981. 147 (1): p. 195-197.
10. Pearson, W. R., Searching protein sequence libraries: comparison of the sensitivity and selectivity of the Smith-Waterman and FASTA algorithms. Genomics, 1991. 11 (3): p. 635-650.
11. Pearson, W. R. and D. J. Lipman, Improved tools for biological sequence comparison. Proceedings of the National Academy of Sciences, 1988. 85 (8): p. 2444-2448.
12. Johnson, L. S., S. R. Eddy, and E. Portugaly, Hidden Markov model speed heuristic and iterative HMM search procedure. BMC bioinformatics, 2010. 11 (1): p. 1-8.
13. Goldenzweig, A., et al., Automated structure-and sequence-based design of proteins for high bacterial expression and stability. Molecular cell, 2016. 63 (2): p. 337-346.
14. Sambrook, J. and D. Russell, Molecular cloning: A laboratory manual. Mol. Cloning A Lab. Man, 2001.
15. Dietrich, L. E., et al., The phenazine pyocyanin is a terminal signalling factor in the quorum sensing network of Pseudomonas aeruginosa. Molecular microbiology, 2006. 61 (5): p. 1308-1321.
16. Sullivan, N. L., et al., Quantifying the dynamics of bacterial secondary metabolites by spectral multiphoton microscopy. ACS chemical biology, 2011. 6 (9): p. 893-899.
17. Yang, Z.-J., et al., Isolation, identification, and degradation characteristics of phenazine-1-carboxylic acid-degrading strain Sphingomonas sp. DP58. Current microbiology, 2007. 55 (4): p. 284-287.
18. Chen, K., et al., Metabolic degradation of phenazine-1-carboxylic acid by the strain Sphingomonas sp. DP58: the identification of two metabolites. Biodegradation, 2008. 19 (5): p. 659-667.
19. Möker, N., C. R. Dean, and J. Tao, Pseudomonas aeruginosa increases formation of multidrug-tolerant persister cells in response to quorum-sensing signaling molecules. Journal of bacteriology, 2010. 192 (7): p. 1946-1955.
20. Wang. Y. and D. K. Newman, Redox reactions of phenazine antibiotics with ferric (hydr) oxides and molecular oxygen. Environmental Science & Technology, 2008. 42 (7): p. 2380-2386.
21. Wang, Y. and D. K. Newman, Redox reactions of phenazine antibiotics with ferric (hydr) oxides and molecular oxygen. Environmental science & technology, 2008. 42 (7): p. 2380-2386.
22. Fultz, M. L. and R. A. Durst, Mediator compounds for the electrochemical study of biological redox systems: a compilation. Analytica Chimica Acta, 1982. 140 (1): p. 1-18.
23. Hernandez, M. E. and D. K. Newman, Extracellular electron transfer. Cellular and Molecular Life Sciences, 2001. 58 (11): p. 1562-1571.
24. Hernandez, M. and D. Newman, Extracellular electron transfer. Cellular and Molecular Life Sciences CMLS, 2001. 58 (11): p. 1562-1571.
25. Michaelis, L. and E. S. Hill, POTENTIOMETRIC STUDIES ON SEMIQUINONES. Journal of the American Chemical Society, 1933. 55: p. 1481-1494.
26. Michaelis, L. and E. S. Hill, THE VIOLOGEN INDICATORS. THE JOURNAL OF GENERAL PHYSIOLOGY, 1933. 16: p. 859-873.
27. Michaelis, L. and E. S. Hill, The viologen indicators. Journal of General Physiology, 1933. 16 (6): p. 859-873.
28. Wang, Y., S. E. Kern, and D.K. Newman, Endogenous phenazine antibiotics promote anaerobic survival of Pseudomonas aeruginosa via extracellular electron transfer. Journal of bacteriology, 2010. 192 (1): p. 365-369.
29. Heydorn, A., et al., Quantification of biofilm structures by the novel computer program COMSTAT. Microbiology, 2000. 146 (10): p. 2395-2407.
30. Banin, E., M. L. Vasil, and E.P. Greenberg, Iron and Pseudomonas aeruginosa biofilm formation. Proceedings of the National Academy of Sciences, 2005. 102 (31): p. 11076-11081.
31. Wang, Y., et al., Phenazine-1-carboxylic acid promotes bacterial biofilm development via ferrous iron acquisition. Journal of bacteriology, 2011. 193 (14): p. 3606-3617.
32. Friedheim, E.A., Pyocyanin, an accessory respiratory enzyme. The Journal of experimental medicine, 1931. 54 (2): p. 207.
33. Kidani, Y., K. Inagaki, and H. Koike, Studies on metal chelate compounds of phenazine derivatives. 8. Metal complex of 1-hydroxyphenazine (author's transl). Yakugaku zasshi: Journal of the Pharmaceutical Society of Japan, 1973. 93 (9): p. 1089-1093.
34. Kidani, Y., Studies on Metal Chelate Compounds of Phenazine Derivatives. I.

Spectrophotometric Studies on Copper Chelate Compounds of 1-Hydroxyphenazine and its Di-N-oxide. Chemical and Pharmaceutical Bulletin, 1958. 6 (5): p. 556-562.
35. Orenstein, D. M., Cystic fibrosis: A guide for patient and family 4th ed. 4th ed. 2011: Lippincott Williams & Wilkins.
36. Miroux, B. and J. E. Walker, Over-production of proteins in Escherichia coli: mutant hosts that allow synthesis of some membrane proteins and globular proteins at high levels. Journal of molecular biology, 1996. 260 (3): p. 289-298.
37. VanDrisse, C. and J. Escalante-Semerena, New high-cloning-efficiency vectors for complementation studies and recombinant protein overproduction in Escherichia coli and Salmonella enterica. Plasmid, 2016. 86: p. 1-6.
38. Galloway, N. R., et al., Rapid cloning for protein crystallography using type IIS restriction enzymes. Crystal growth & design, 2013. 13 (7): p. 2833-2839.
39. Blommel, P. G., et al., Enhanced bacterial protein expression during auto-induction obtained by alteration of lac repressor dosage and medium composition. Biotechnology progress, 2007. 23 (3): p. 585-598.
40. Netzer, R., et al., Ultrahigh specificity in a network of computationally designed protein-interaction pairs. Nature communications, 2018. 9 (1): p. 1-13.
41. Khersonsky, O., et al., Automated design of efficient and functionally diverse enzyme repertoires. Molecular cell, 2018. 72 (1): p. 178-186. e5.
42. Cheluvappa. R., Standardized chemical synthesis of Pseudomonas aeruginosa pyocyanin. MethodsX, 2014. 1: p. 67-73.
43. O'Toole, G. A., Microtiter dish biofilm formation assay. Journal of visualized experiments: JOVE, 2011 (47).
44. Spero, M. A. and D.K. Newman, Chlorate specifically targets oxidant-starved, antibiotic-tolerant populations of Pseudomonas aeruginosa biofilms. MBio, 2018. 9 (5): p. e01400-18.
45. Palmer, K. L., L. M. Aye, and M. Whiteley, Nutritional cues control Pseudomonas aeruginosa multicellular behavior in cystic fibrosis sputum. Journal of bacteriology, 2007. 189 (22): p. 8079-8087.
46. Warszawski, S., et al., Optimizing antibody affinity and stability by the automated design of the variable light-heavy chain interfaces. PLOS computational biology, 2019. 15 (8): p. e1007207.
47. Cornforth, D. M., et al., Quantitative framework for model evaluation in microbiology research using Pseudomonas aeruginosa and cystic fibrosis infection as a test case. MBio, 2020. 11 (1): p. e03042-19.
48. Walters III, M.C., et al., Contributions of antibiotic penetration, oxygen limitation, and low metabolic activity to tolerance of Pseudomonas aeruginosa biofilms to ciprofloxacin and tobramycin. Antimicrobial agents and chemotherapy, 2003. 47 (1): p. 317-323.
49. Meylan, S., et al., Carbon sources tune antibiotic susceptibility in Pseudomonas aeruginosa via tricarboxylic acid cycle control. Cell chemical biology, 2017. 24 (2): p. 195-206.
50. Ruddy, J., et al., Sputum tobramycin concentrations in cystic fibrosis patients with repeated administration of inhaled tobramycin. Journal of aerosol medicine and pulmonary drug delivery, 2013. 26 (2): p. 69-75.
51. Kragh, K. N., et al., Role of multicellular aggregates in biofilm formation. MBio, 2016. 7 (2): p. e00237-16.
52. Stewart, P.S., Diffusion in biofilms. Journal of bacteriology, 2003. 185 (5): p. 1485-1491.
53. Bellin, D. L., et al., Electrochemical camera chip for simultaneous imaging of multiple metabolites in biofilms. Nature communications, 2016. 7 (1): p. 1-10.
54. Tang. X. X., et al., Acidic pH increases airway surface liquid viscosity in cystic fibrosis. The Journal of clinical investigation, 2016. 126 (3): p. 879-891.
55. Ramos, I., et al., Phenazines affect biofilm formation by Pseudomonas aeruginosa in similar ways at various scales. Research in microbiology, 2010. 161 (3): p. 187-191.
56. Saunders, S. H., et al., Extracellular DNA promotes efficient extracellular electron transfer by pyocyanin in Pseudomonas aeruginosa biofilms. Cell, 2020. 182 (4): p. 919-932. e19.
57. Dietrich, L. E., et al., Bacterial community morphogenesis is intimately linked to the intracellular redox state. Journal of bacteriology, 2013. 195 (7): p. 1371-1380.
58. Schiessl, K. T., et al., Phenazine production promotes antibiotic tolerance and metabolic heterogeneity in Pseudomonas aeruginosa biofilms. Nature communications, 2019. 10 (1): p. 1-10.
59. Zhu, K., et al., Universal antibiotic tolerance arising from antibiotic-triggered accumulation of pyocyanin in Pseudomonas aeruginosa. PLOS biology, 2019. 17 (12): p. e3000573.
60. Meirelles, L. A., et al., Bacterial defenses against a natural antibiotic promote collateral resilience to clinical antibiotics. PLOS biology, 2021. 19 (3): p. e3001093.
61. Glasser, N. R., S. E. Kern, and D.K. Newman, Phenazine redox cycling enhances anaerobic survival in Pseudomonas aeruginosa by facilitating generation of ATP and a proton-motive force. Molecular microbiology, 2014. 92 (2): p. 399-412.
62. Meirelles, L. A. and D. K. Newman, Both toxic and beneficial effects of pyocyanin contribute to the lifecycle of Pseudomonas aeruginosa. Molecular microbiology, 2018. 110 (6): p. 995-1010.
63. Sezonov, G., D. Joseleau-Petit, and R. d′Ari, Escherichia coli physiology in Luria-Bertani broth. Journal of bacteriology, 2007. 189 (23): p. 8746-8749.
64. Jo, J., et al., Interdependency of respiratory metabolism and phenazine-associated physiology in Pseudomonas aeruginosa PA14. Journal of bacteriology, 2020. 202 (4): p. e00700-19.

1.-4. (canceled)

5. An engineered pyocyanin demethylase or derivative thereof, wherein the pyocyanin demethylase comprises a protein having sequence of SEQ ID NO: 2
modified to include at least two replacements in SEQ ID NO:2 selected from
A24 with L, N, or V
144 with I, E, K, L, Q, R, T or V
A58 with C, I, T or V
T62 with V
M70 with M, C, F, I, K, R, V or Y
A100 with A, C, S, T, or V
K112 with K, S or T
the engineered pyocyanin demethylase or a derivative thereof configured to be capable of demethylating pyocyanin and/or a pyocyanin-like phenazine of formula (III) 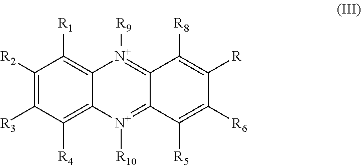
where R₁-R₉ are independently selected from hydrogen, hydroxy, alkoxy, alkyl, alkenyl, alkynyl, aryl, heteroaryl, and acyl, R₁₀ is a methyl group, and one of R₁-R₉ is a negatively charged substituent,
wherein the amino acid sequence of SEQ ID NO: 2 has demethylating residues D39, D43, H92, E125, and Y127,
wherein the derivative has at least 40% sequence identity with SEQ ID NO:2, and comprises the demethylating residues:
D, E or Y at position 39;
D, E, or Y at position 42;
H, R, K at position 92;
D, E, or Y at position 125; and
D, E or Y at position 127
wherein the derivative has the ability to demethylate the pyocyanin-like phenazines of formula (III).

6. The engineered pyocyanin demethylase or derivative thereof of claim 5, wherein the engineered pyocyanin demethylase has replacements in positions 44, 58, 62, 70 and 100 of SEQ ID NO:2, or replacements in positions 44, 58, 70, 100 and 112 of SEQ ID NO:2, or replacements in positions 44, 58, 62, 100 and 112 of SEQ ID NO: 2, or replacements in positions 24, 44, 58, 70 and 100 of SEQ ID NO: 2.

7. The engineered pyocyanin demethylase or derivative thereof of claim 5, wherein the engineered pyocyanin demethylase has at least two of a replacement in position 44 selected from 144T, I44K, I44L, I44R, a replacement in position 58 selected from A58V, and A58I, a replacement in position 70 selected from M70V and M70T, a replacement in position 100 selected from A100V and A100T, replacement K112T in position 112, replacement T62V in position 62 and replacement A24N, in position 24.

8. The engineered pyocyanin demethylase or derivative thereof of claim 5, wherein the engineered pyocyanin demethylase is
PodA2₃₀₋₁₆₂ having SEQ ID NO: 2 including replacements 144T, A58V, T62V, M70V and A100V, or a derivative thereof,
PodA3₃₀₋₁₆₂ having SEQ ID NO: 2 including replacements I44K, A58, T62V, M70V, A100V, or a derivative thereof,
PodA5₃₀₋₁₆₂ having SEQ ID NO: 2 including replacements I44L, A581, M70V, A100V, K112T, or a derivative thereof,
PodA6₃₀₋₁₆₂ having SEQ ID NO: 2 including replacements I44R, A58V, T62V, M70V, A100T, or a derivative thereof,
PodA7₃₀₋₁₆₂ having SEQ ID NO: 2 including replacements I44L, A581, T62V, M70T, and A100V, or a derivative thereof,
PodA8₃₀₋₁₆₂ having SEQ ID NO: 2 including replacements A24N, I44R, A58V, T62V, A100V, or a derivative thereof,
PodA9₃₀₋₁₆₂ having SEQ ID NO: 2 including replacements I44K, A58V, T62V, A100V and K112T, or a derivative thereof, or
PodA10₃₀₋₁₆₂ having SEQ ID NO: 2 including replacements A24N, 144T, A58V, M70V, A100T, or a derivative thereof.

9. The engineered pyocyanin demethylase or derivative thereof of claim 5, wherein the pyocyanin demethylase has a deletion from residue V1 to residue P11 and the engineered pyocyanin demethylase has sequence SEQ ID NO: 3
in which
residue D28 corresponds to demethylating residue D39 of SEQ ID NO: 2,
residue D32 corresponds to demethylating residue D43 of SEQ ID NO: 2,
residue H81corresponds to demethylating residue H92 of SEQ ID NO: 2,
residue E114 corresponds to demethylating residue E125 of SEQ ID NO: 2, and
residue Y116 corresponds to demethylating residue Y127 of SEQ ID NO: 2
and in which
residue A13 corresponds to residue A24 of SEQ ID NO: 2,
residue 133 corresponds to residue 1144 of SEQ ID NO: 2,
residue A47 corresponds to residue A58 of SEQ ID NO: 2,
residue T51 corresponds to residue T62 of SEQ ID NO: 2,
residue M59 corresponds to residue M70 of SEQ ID NO: 2,
residue A89 corresponds to residue A100 of SEQ ID NO: 2, and
residue K101 corresponds to K112 of SEQ ID NO: 2.

10. The engineered pyocyanin demethylase or derivative thereof of claim 9, wherein the engineered demethylase has replacements in positions 33, 47, 51, 59 and 89 of SEQ ID NO: 3, or in positions 33, 47, 59, 89 and 101 of SEQ ID NO: 3, or in positions 33, 47, 51, 89 and 101 of SEQ ID NO: 3, or in positions 13, 33, 47, 59 and 89 of SEQ ID NO: 3.

11. The engineered pyocyanin demethylase or derivative thereof of claim 9, wherein the engineered demethylase has at least two of a replacement in position 33 selected from 133T, I33K, I33L, I33R, a replacement in position 47 selected from A47V, and A471, a replacement in position 59 selected from M59V and M59T, a replacement in position 89 selected from A89V and A89T, the replacement K101T in position 101, the replacement T51V in position 51 and the replacement A13N in position 13 of SEQ ID NO: 3.

12. The engineered pyocyanin demethylase or derivative thereof of claim 9, wherein the engineered demethylase is
PodA2₄₁₋₁₆₂ having SEQ ID NO: 3 including replacements 133T, A47V, T51V, M59V and A89V, or a derivative thereof,
PodA3₄₁₋₁₆₂ having SEQ ID NO: 3 including replacements I33K, A47V, T51V, M59V, A89V, or a derivative thereof,
PodA5₄₁-162 having SEQ ID NO: 3 including replacements I33L, A471, M59V, A89V, K101T, or a derivative thereof,
PodA6 ₄₁₋₁₆₂ having SEQ ID NO: 3 including replacements I33R, A47V, T51V, M59V, A89T, or a derivative thereof,
PodA7₄₁₋₁₆₂ having SEQ ID NO: 3 including replacements I33L, A471, T51V, M59T, and A89V, or a derivative thereof,
PodA8 ₄₁₋₁₆₂ having SEQ ID NO: 3 including replacements A13N, I33R, A47V, T51V, A89V, or a derivative thereof,
PodA9₄₁₋₁₆₂ having SEQ ID NO: 3 including replacements I33K, A47V, T51V, A89V and K101T, or a derivative thereof, or
PodA10₄₁₋₁₆₂ having SEQ ID NO: 3 including replacements A13N, I33T, A47V, M59V, A89T, or a derivative thereof.

13. (canceled)

14. A phenazine degrading agent comprising the engineered pyocyanin demethylase or derivative thereof according to claim 5.

15. A method to interfere with viability of phenazine producing bacteria, the method comprising
contacting the phenazine producing bacteria with one or more pyocyanin demethylases or derivative thereof of claim 5, alone or in combination with an antibiotic and/or other antimicrobial for a time and under conditions to reduce survivability and/or antibiotic resistance of the bacteria.

16. (canceled)

17. The method of claim 15, wherein the engineered pyocyanin demethylase comprises the engineered pyocyanin demethylase having the replacements in positions 44, 58, 62, 70 and 100 of SEQ ID NO:2, or the replacements in positions 44, 58, 70, 100 and 112 of SEQ ID NO:2, or the replacements in positions 44, 58, 62, 100 and 112 of SEQ ID NO: 2, or the replacements in positions 24, 44, 58, 70 and 100 of SEQ ID NO: 2.

18. The method of claim 15, wherein the engineered pyocyanin demethylase comprises the engineered pyocyanin demethylase having the replacements in positions 33, 47, 51, 59 and 89 of SEQ ID NO: 3, or the replacements in positions 33, 47, 59, 89 and 101 of SEQ ID NO: 3, or the replacements in positions 33, 47, 51, 89 and 101 of SEQ ID NO: 3, or the replacements in positions 13, 33, 47, 59 and 89 of SEQ ID NO: 3.

19. The method of claim 15, wherein the engineered pyocyanin demethylase comprises
PodA10₃₀₋₁₆₂ having SEQ ID NO: 2 including replacements A24N, 144T, A58V, M70V, A100T, or a derivative thereof and/or
PodA10₄₁₋₁₆₂ having SEQ ID NO: 3 including replacements A13N, 133T, A47V, M59V, A89T, or a derivative thereof.

20. The method of claim 15, wherein the one or more phenazine degrading agents are capable of degrading pyocyanin-like phenazine of formula III, wherein pyocyanin-like phenazines comprise phenazines of formula III wherein at least one of R₁-R₈ is hydroxy group, or a methoxy group.

21. The method of claim 15, wherein the antibiotic comprises one or more aminoglycosides.

22. The method of claim 21, wherein the antibiotic comprises one or more of an aminoglycoside of 4,6-disubstituted deoxystreptamine sub-class of aminoglycosides, an aminoglycoside of 4,5-disubstituted sub-class, and a non-deoxystreptamine aminoglycoside.

23. The method of claim 21, wherein the antibiotic comprises one or more of Kanamycin A Amikacin, Tobramycin, Dibekacin, Gentamicin, Sisomicin, Netilmicin, Neomycins B, C, Streptomycin and Plazomicin.

24. The method of claim 15, wherein the antibiotic is selected from the group consisting of Amoxicillin and clavulanic acid, Methicillin, oxacillin, nafcillin, cloxacillin, dicloxacillin, cabenicillin, ticarcillin, piperacillin, mezlocillin, azlocillin, ticarcillin and clavulanic acid, piperacillin and tazobactam, cephalexin, cefdinir, cefprozil, cefaclor, cefuroxime, sulfisoxazole, erythromycin/sulfisoxazole, tobramycin, amikacin, gentamicin, erythromycin, clarithromycin, azithromycin, tetracycline, doxycycline, minocycline, tigecycline, ciprofloxacin, levofloxacin, vancomycin, linezolid, imipenem, meripenem, and aztreonam.

25. The method of claim 15, wherein the phenazine producing bacteria is selected from the group consisting of Staphylococcus aureus, Pseudomonas, Burkholderia cepacia, and mycobacteria.

26. The method of claim 15, wherein the phenazine producing bacteria is in a biofilm.

27. A method for inhibiting bacteria biofilm formation and/or disrupting mature biofilm in a medium, the method comprising:
contacting the bacterial biofilm with one or more pyocyanin demethylases or derivatives thereof of claim 5, alone or in combination with an antibiotic and/or other antimicrobial for a time and under conditions thus reducing survivability and/or antibiotic resistance of the bacterial biofilm.

28. The method of claim 27, wherein the bacteria biofilm comprises one or more of Staphylococcus aureus, Pseudomonas, Burkholderia cepacia, and mycobacteria.

29. A system to interfere with viability of phenazine producing bacteria, the system comprising
1) one or more pyocyanin demethylases or derivative thereof of claim 5, and
2) one or more antibiotics, and/or one or more other antimicrobials,
for simultaneous combined or sequential use in a method comprising:
contacting the bacterial biofilm with the one or more pyocyanin demethylases or derivatives thereof alone or in combination with an antibiotic and/or other antimicrobial for a time and under conditions thus reducing survivability and/or antibiotic resistance of the bacterial biofilm.

30. A method for treating and/or preventing a bacterial infection by phenazine producing bacteria in an individual, the method comprising
administering to the individual an effective amount of one or more pyocyanin demethylases or derivative thereof of claim 5, alone or in combination with an antibiotic and/or other antimicrobial.

31. A system for treating and/or preventing a bacterial infection by a phenazine producing bacteria in an individual, the system comprising
1) one or more pyocyanin demethylases or derivative thereof of claim 5, and
2) one or more antibiotics and/or one or more other antimicrobials,
for simultaneous combined or sequential use in a method for treating and/or preventing a bacterial infection by phenazine producing bacteria in the individual.

32. An antimicrobial comprising one or more pyocyanin demethylases of claim 5, in an amount suitable to reduce antibiotic resistance and/or survivability of phenazine producing bacteria.

33. A composition comprising one or more pyocyanin demethylases of claim 5, together with a compatible vehicle.

34. The composition of claim 33, wherein the vehicle is a pharmaceutically acceptable vehicle and the composition is a pharmaceutical composition.

35. A method for inhibiting bacteria biofilm formation and/or disrupting mature biofilm in a medium, the method comprising:
administering to the medium comprising the bacterial biofilm an effective amount of 1-hydroxyphenazine alone or in combination with one or more pyocyanin demethylase or derivatives thereof of claim 5, an antibiotic and/or other antimicrobial for a time and under conditions thus reducing survivability and/or antibiotic resistance of the bacterial biofilm.


SEQUENCE LISTING (10 sequences)

SEQ ID NO: 1 - protein, 162 aa, Mycobacterium fortuitum
MTGKTKPAIIGGVAITALAAAGLGVWLFTDGRGGRSTTEPVTMTLDVKNDQVAKHDFGKPGMDVGDMDIFSDILSVDGKQVGYDGGACFFTNVTPDNPMTYCELTIHLDAGEIFARSLTPHTLAPFTMAITGGTGEYANSKGELTVSGVATPDEKYELKLTK

SEQ ID NO: 2 - protein, 141 aa, synthetic construct
  source 1..141: note = Synthetic polypeptide
MDGRGGRSTTEPVTMTLDVKNDQVAKHDFGKPGMDVGDMDIFSDILSVDGKQVGYDGGACFFTNVTPDNPMTYCELTIHLDAGEIFARSLTPHTLAPFTMAITGGTGEYANSKGELTVSGVATPDEKYELKLTKAENLYFQ

SEQ ID NO: 3 - protein, 122 aa, synthetic construct
  source 1..122: note = Synthetic polypeptide
VTMTLDVKNDQVAKHDFGKPGMDVGDMDIFSDILSVDGKQVGYDGGACFFTNVTPDNPMTYCELTIHLDAGEIFARSLTPHTLAPFTMAITGGTGEYANSKGELTVSGVATPDEKYELKLTK

SEQ ID NO: 4 - protein, 123 aa, synthetic construct
  source 1..123: note = Synthetic polynucleotide
MVTMTLDVKNDQVNKHDFGKPGMDVGDMDIFSDTLSVDGKQVGYDGGVCFFTNVTPDNPVTYCELTIHLDAGEIFARSLTPATLAPFTMTITGGTGEYANSKGELTVSGVATPDEKYELKLTK

SEQ ID NO: 5 - DNA, 41 bp, synthetic construct
  source 1..41: mol_type = other DNA; note = Synthetic polynucleotide
nngctcttcnttcatggacggtcgcggcggccggagtacaa

SEQ ID NO: 6 - DNA, 43 bp, synthetic construct
  source 1..43: mol_type = other DNA; note = Synthetic polynucleotide; note = Synthetic polynucleotide
nngctcttcntaatcatttcgtcagtttcaattcgtacttctc

SEQ ID NO: 7 - DNA, 29 bp, synthetic construct
  source 1..29: mol_type = other DNA; note = Synthetic polynucleotide
gccctgaaaatacaggttttcactagttg

SEQ ID NO: 8 - DNA, 25 bp, synthetic construct
  source 1..25: mol_type = other DNA; note = Synthetic polynucleotide
taagaattctcgagctcccgggatc

SEQ ID NO: 9 - DNA, 41 bp, synthetic construct
  source 1..41: mol_type = other DNA; note = Synthetic polynucleotide
attacgatatcccaactagtgaaaacctgtattttcagggc

SEQ ID NO: 10 - DNA, 40 bp, synthetic construct
  source 1..40: mol_type = other DNA; note = Synthetic polynucleotide
tgctcagcggccgcggatcccgggagctcgagaattctta